(19) United States
(12) Patent Application Publication
(10) Pub. No.: US 2021/0246455 A1
(43) Pub. Date: Aug. 12, 2021

(54) MULTIPLEX ASSAY

(71) Applicant: Cairn Biosciences, Inc., San Francisco, CA (US)

(72) Inventors: M. Susana G. DE ABREU RIBEIRO, San Francisco, CA (US); Catherine I. LACAYO, Fremont, CA (US); Mary J. C. LUDLAM, San Francisco, CA (US)

(21) Appl. No.: 16/614,297

(22) PCT Filed: May 15, 2018
(86) PCT No.: PCT/US2018/032834

§ 371 Date: Nov. 15, 2019

Related U.S. Application Data
(60) Provisional application No. 62/507,169, filed on May 16, 2017.

(51) Int. Cl. C12N 15/67; C12N 15/10; G01N 33/50

(52) U.S. Cl. CPC C12N 15/67; C12N 15/1093; G01N 33/5023; G01N 2500/10; C12N 2830/20; C12N 2840/20; C12N 2840/002

(57) ABSTRACT

The present invention provides assays for profiling two or more polypeptides in live cells. In some embodiments, the invention provides multicistronic reporter vectors, acceptor cells for receiving multicistronic reporter vectors, and multireporter cells. Methods of making multicistronic reporter vectors, acceptor cells for receiving multicistronic reporter vectors, and multireporter cells are provided. Libraries and kits comprising multicistronic reporter vectors, acceptor cells for receiving multicistronic reporter vectors, and multireporter cells are provided. Methods of profiling/assaying the multireporter cells and multireporter cell libraries are provided.

### CROSS-REFERENCE TO RELATED APPLICATIONS

This application is a national stage application under 35 U.S.C. § 371 of International Application No. PCT/US2018/032834, filed internationally May 15, 2018, which claims the benefit of U.S. Provisional Patent Application No.62/507,169 filed May 16, 2017, the disclosure of which are hereby incorporated by reference in their entirety.

### STATEMENT REGARDING FEDERALLY SPONSORED RESEARCH OR DEVELOPMENT

This invention was made with government support under grant nos. 1448764 and 1632576 awarded by the National Science Foundation and contracts HHSN271201600006C, HHSN271201500028C and HHSN271201600021C from the National Institutes of Health. The government has certain rights in the invention.

### BACKGROUND OF THE INVENTION

Ascertaining cellular responses to internal or external stimuli necessitates visualization and monitoring of key players and pathways. Current methods of visualizing such responses are inefficient, costly and provide only a snapshot. For example, drug discovery and/or toxicological evaluation of drug candidates is traditionally carried out using in vivo preclinical animal models that are low-throughput, costly, inadequately predictive of toxicity in humans and offer little insight into the mechanisms of compound toxicity. The throughput of these traditional approaches is far outpaced by the rate at which new chemicals are generated, be they compounds developed for pharmaceutical, agricultural or other purposes such as nutrition, cosmetics, personal care, etc. The need for these new chemicals to be evaluated for efficacy or for the risks they may pose to human health mandates the development of new screening tools that can offer meaningful insights into the mechanisms associated with such compounds at a throughput compatible of evaluating hundreds of thousands of compounds per week. Ideally new screening tools would be capable of pinpointing which aspects of cellular physiology are perturbed by a particular chemical and measuring the resulting cellular response. These needs can be addressed by an appropriately configured live-cell approach, which is the focus of the present invention.

All references cited herein, including patent applications and publications, are incorporated by reference in their entirety.

The invention provides compositions and methods for multiplex live cell screening (e.g., live cell high-content screening (LC-HCS)) in a variety of cells, including, immortalized cells, primary cells, human stem cells, human iPSC cells and human iPSC-derived cells, as a model for drug-discovery and toxicology screens requiring high-throughput technologies.

In some aspects, the invention provides a multicistronic reporter vector comprising: a promoter operably linked to an open reading frame, wherein the open reading frame comprises two or more cistrons, and wherein expression of the open reading frame in a cell yields separate component polypeptide products from each cistron; wherein each cistron comprises a multiple cloning site (MCS) and nucleic acid encoding a reporter vector, and wherein each cistron encodes a different reporter polypeptide; and wherein expression of two or more nucleic acids encoding polypeptides inserted into the two or more multiple cloning sites and fused to the reporter polypeptides is essentially stoichiometric. In some embodiments, the cistrons are separated from one another by nucleic acid encoding one or more self-cleaving peptide and/or one or more internal ribosome entry site (IRES). In some embodiments, the one or more self-cleaving peptides is a viral self-cleaving peptide. In some embodiments, the one or more viral self- cleaving peptides is one or more 2A peptides. In some embodiments, one or more 2A peptides is a T2A peptide, a P2A peptide, an E2A peptide or a F2A peptide. In some embodiments, the reporter polypeptide further comprises one or more nucleic acids encoding a peptide linker between one or more of the reporter polypeptides and one or more of the self-cleaving peptides. In some embodiments, the peptide linker comprises the sequence Gly-Ser-Gly. In some embodiments, the reporter polypeptide is a fluorescent reporter polypeptide. In some embodiments, the reporter polypeptide for each cistron is selected from GFP, EGFP, Emerald, Citrine, Venus, mOrange, mCherry, TagBFP, mTurquoise, Cerulean, UnaG, dsRed, eqFP611, Dronpa, RFP, TagRFPs, TdTomato, KFP, EosFP, Dendra, IrisFP, iRFPs and smURFP.

In some embodiments, the multicistronic reporter vector comprises a promoter operably linked to an open reading frame, wherein the open reading frame comprises a first cistron and a second cistron, wherein each cistron comprises 5′ to 3′ nucleic acid comprising a MCS, nucleic acid encoding a reporter polypeptide, and nucleic acid encoding a linker peptide; wherein the first cistron and the second cistron are separated by nucleic acid encoding a viral cleavage peptide. In some embodiments, the multicistronic reporter vector comprises a promoter operably linked to an open reading frame, wherein the open reading frame comprises a first cistron, a second cistron and a third cistron, wherein each cistron comprises 5′ to 3′ nucleic acid comprising a MCS, nucleic acid encoding a reporter polypeptide, and nucleic acid encoding a linker peptide; wherein the first cistron and the second cistron are separated by nucleic acid encoding a first viral cleavage peptide and the second cistron and the third cistron are separated by nucleic acid encoding a second viral cleavage peptide.

In some embodiments, the multicistronic reporter vector comprises a promoter operably linked to an open reading frame, wherein the open reading frame comprises a first cistron, a second cistron, a third cistron and a fourth cistron, wherein each cistron comprises 5′ to 3′ nucleic acid comprising a MCS, nucleic acid encoding a reporter polypeptide nucleic acid encoding a linker peptide; wherein the first cistron and the second cistron are separated by nucleic acid encoding a first viral cleavage peptide, the second cistron and the third cistron are separated by nucleic acid encoding a second viral cleavage peptide the third cistron and the fourth cistron are separated by nucleic acid encoding a third viral cleavage peptide. In some embodiments, the multicistronic reporter vector comprises a promoter operably linked to an open reading frame, wherein the open reading frame comprises a first cistron, a second cistron, a third cistron and a fourth cistron, wherein each cistron comprises 5′ to 3′ nucleic acid comprising a MCS, nucleic acid encoding a reporter polypeptide, nucleic acid encoding a linker peptide; wherein the first cistron and the second cistron are separated by nucleic acid encoding a first viral cleavage peptide, the second cistron and the third cistron are separated by nucleic acid encoding a second viral cleavage peptide the third cistron and the fourth cistron are separated by nucleic acid encoding an IRES.

In some embodiments, the multicistronic reporter vector further comprises one or more inducible elements located between the promoter and open reading frame. In some embodiments, the multicistronic reporter vector comprises two inducible elements. In some embodiments, the inducible element is a Tet operator 2 (TetO2) inducible element.

In some embodiments, the multicistronic reporter vector further comprises a constitutive promoter. In some embodiments, the constitutive promoter is a Cytomegalovirus a (CMV), a Thymidine Kinase (TK), an eF 1-alpha, a Ubiquitin C (UbC), a Phosphoglycerate Kinase (PGK), a CAG promoter, an SV40 promoter, or a human β-actin promoter. In some embodiments, the promoter is an inducible promoter. In some embodiments, the inducible promoter is a tetracycline responsive promoter. In some embodiments, the promoter is a tissue specific promoter. In some embodiments, the tissue specific promoter is specific for cells of heart, blood, muscle, lung, liver, kidney, pancreas, brain, or skin.

In some embodiments, the multicistronic reporter vector further comprises a site-specific recombinase sequence located 3′ to the open reading frame. In some embodiments, the vector further comprises nucleic acid encoding a selectable marker, wherein the nucleic acid encoding the selectable marker is not operably linked to the promoter when the site-specific recombinase sequence has not recombined and is operably linked to the promoter when the site-specific recombinase sequence recombines with its target site-specific recombinase sequence. In some embodiments, the site-specific recombinase sequence is a FRT nucleic acid sequence and/or an attP nucleic acid and/or a loxP nucleic acid sequence.

In some embodiments, the selectable marker of the vector confers resistance to hygromyocin, Zeocin™, puromycin, blasticidin, neomycin or an analog of hygromyocin, Zeocin™, puromycin, blasticidin or neomycin.

In some embodiments, the multicistronic reporter vector comprising nucleic acid encoding one or more polypeptides is inserted in-frame into the one or more MCS. In some embodiments, the one or more polypeptides comprise polypeptides that can be used to profile a single biological pathway, cross-talk between two or more biological pathways, synthetic lethality, cellular homeostasis, organelle homeostasis or a toxicity response.

In some embodiments, the multicistronic reporter vector further comprises one, two or three transcription units comprising a promoter and nucleic acid encoding a transgene located 5′ to the open reading frame comprising two or more cistrons, wherein the reporter vector further comprises a core insulator sequence and a polyA sequence located 3′ to the transcription units and 5′ to the open reading frame comprising two or more cistrons.

In some aspects, the invention provides an acceptor cell for receiving a multicistronic reporter vector, wherein the acceptor cell comprises a recombinant nucleic acid integrated into a specific site in a host cell genome, wherein the recombinant nucleic acid comprises a first promoter operably linked to nucleic acid encoding a fusion polypeptide, wherein the fusion polypeptide comprises a reporter domain and a selectable marker domain, and wherein the nucleic acid comprises a site-specific recombinase nucleic acid sequence located at the 5′ end of the nucleic acid encoding the fusion polypeptide.

In some embodiments, the promoter of the acceptor cell is a constitutive promoter. In some embodiments, the constitutive promoter is a CMV promoter, a TK promoter, an eF1-alpha promoter, a UbC promoter, a PGK promoter, a CAG promoter, an SV40 promoter, or a human β-actin promoter. In some embodiments, the promoter is an inducible promoter. In some embodiments, the inducible promoter is a tetracycline responsive promoter.

In some embodiments, the site-specific recombinase sequence of the acceptor cell is a FRT nucleic acid sequence and/or an attP nucleic acid sequence and/or a loxP nucleic acid sequence.

In some embodiments, the reporter domain of the fusion polypeptide is a fluorescent reporter domain. In some embodiments, the fluorescent reporter domain is selected from GFP, EGFP, Emerald, Citrine, Venus, mOrange, mCherry, TagBFP, mTurquoise, Cerulean, UnaG, dsRed, eqFP611, Dronpa, RFP, TagRFPs, TdTomato, KFP, EosFP, Dendra, IrisFP, iRFPs, and smURFP In some embodiments, the reporter domain of the fusion polypeptide is an mCherry reporter domain.

In some embodiments, the selectable marker domain of the fusion polypeptide confers resistance to hygromycin, Zeocin™, puromycin, blasticidin, neomycin or an analog of hygromycin, Zeocin™, puromycin, blasticidin, neomycin.

In some embodiments, the integrated recombinant nucleic acid of the acceptor cell further comprises nucleic acid encoding a tetracycline repressor polypeptide operably linked to a promoter. In some embodiments, the promoter is a human β-actin promoter or a CAG promoter.

In some embodiments, the recombinant nucleic acid is integrated in an adeno-associated virus S1 (AAVS1) locus, a chemokine (CC motif) receptor 5 (CCRS) locus, a human ortholog of the mouse ROSA26 locus, the hipp11 (H11) locus or the citrate lyase beta like gene locus (CLYBL) of the acceptor cell.

In some embodiments, the acceptor cell is an immortalized cell. In some embodiments, the immortalized cell is a HEK293T cell, an A549 cell, an U2OS cell, an RPE cell, an NPC1 cell, a MCF7 cell, a HepG2 cell, a HaCat cell, a TK6 cell, an A375 cell or a HeLa cell. In some embodiments, the acceptor cell is a pluripotent cell, an induced pluripotent stem cell, or a multipotent cell. In some embodiments, the induced pluripotent stem cell is a WTC-11 cell or a NCRMS cell. In some embodiments, the acceptor cell is a primary cell.

In some aspects, the invention provides a method for generating an acceptor cell for receiving a multicistronic reporter vector, the method comprising introducing a recombinant nucleic acid to a cell wherein the recombinant nucleic acid comprises 5′ to 3′ a first nucleic acid for targeting homologous recombination to a specific site in the cell, a first promoter, site-specific recombinase nucleic acid, nucleic acid encoding a first reporter polypeptide and a selectable marker, a second nucleic acid for targeting homologous recombination to a specific site in the cell, a second promoter and nucleic acid encoding a second reporter polypeptide, wherein expression of the first reporter polypeptide without expression of the second reporter polypeptide indicates targeting integration of the recombinant nucleic acid to the specific site in the cellular genome and expression of the first and second reporter polypeptides indicates random integration in the cellular genome. In some embodiments, the recombinant nucleic acid further comprises nucleic acid encoding a tetracycline repressor operably linked to a promoter 5′ to the second nucleic acid for targeting homologous recombination.

In some embodiments, the recombinant nucleic acid is integrated into the genome of the acceptor cell using: a) an RNA guided recombination system comprising a nuclease and a guide RNA, b) a TALEN endonuclease, or c) a ZFN endonuclease.

In some embodiments, cells expressing the first reporter polypeptide but not expressing the second reporter polypeptide are selected.

In some embodiments, the site-specific recombinase nucleic acid introduced to the acceptor cell is a FRT nucleic acid sequence and/or an attP nucleic acid sequence and/or a loxP nucleic acid sequence.

In some embodiments, the first reporter polypeptide introduced to the acceptor cell is fluorescent polypeptide and the second reporter polypeptide introduced to the acceptor cell is a different fluorescent polypeptide. In some embodiments, the fluorescent polypeptide is selected from GFP, EGFP, Emerald, Citrine, Venus, mOrange, mCherry, TagBFP, mTurquoise, Cerulean, UnaG, dsRed, eqFP611, Dronpa, RFP, TagRFPs, TdTomato, KFP, EosFP, Dendra, IrisFP, iRFPs, and smURFP. In some embodiments, the first reporter polypeptide is an mCherry reporter and the second reporter polypeptide is GFP.

In some embodiments, the selectable marker introduced to the acceptor cell confers resistance to hygromycin, Zeocin™, puromycin, blasticidin, neomycin or an analog of hygromycin, Zeocin™, puromycin, blasticidin, neomycin.

In some embodiments, the first promoter is a CMV promoter, a TK promoter, an eF1-alpha promoter, a UbC promoter, a PGK promoter, a CAG promoter, an SV40 promoter, or a human β-actin promoter and the second promoter is a CMV promoter, a TK promoter, an eF1-alpha promoter, a UbC promoter, a PGK promoter, a CAG promoter, an SV40 promoter, or a human β-actin promoter. In some embodiments, the first nucleic acid for targeting homologous recombination and the second nucleic acid for targeting homologous recombination target recombination to an AAVS1 locus, a CCR5 locus, a human ortholog of the mouse ROSA26 locus, a H11 locus or a CLYBL locus.

In some embodiments, the acceptor site is introduced to an immortalized cell. In some embodiments, the immortalized cell is a HEK293T cell, an A549 cell, an U2OS cell, an RPE cell, an NPC1 cell, a MCF7 cell, a HepG2 cell, a HaCat cell, a TK6 cell, an A375 cell or a HeLa cell. In some embodiments, the acceptor cite is introduced to the cell is a pluripotent cell, an induced pluripotent stem cell, or a multipotent cell. In some embodiments, the acceptor cite is introduced to a primary cell.

In some embodiments, the invention provides an acceptor cell line generated with the methods described herein.

In some aspects, the invention provides a multireporter cell comprising any acceptor cell described herein in which any multicistronic reporter vector described herein has integrated into the genome of the acceptor cell. In some embodiments, the multicistronic reporter vector has integrated into AAV locus of the acceptor cell.

In some aspects, the invention provides a multireporter cell, wherein the reporter cell comprises a multicistronic reporter construct, wherein the multicistronic reporter construct comprises a promoter operably linked to an open reading frame, wherein the open reading frame comprises two or more cistrons; wherein each cistron comprises a nucleic acid encoding a different transgene product fused to a different reporter polypeptide, wherein expression of the open reading frame in a cell yields separate component polypeptide products from each cistron; and wherein expression of the transgene products is essentially stoichiometric. In some embodiments, the cistrons are separated from one another by nucleic acid encoding one or more self-cleaving peptide and/or one or more internal ribosome entry site (IRES). In some embodiments, each of the reporter polypeptides is a fluorescent reporter polypeptide. In some embodiments, the one or more self-cleaving peptides is one or more 2A peptides. In some embodiments, the nucleic acid encoding the transgene product fused to the reporter polypeptide further comprises one or more nucleic acids encoding a peptide linker between the reporter polypeptide and a viral self-cleaving peptide. In some embodiments, the peptide linker comprises the sequence Gly-Ser-Gly.

In some embodiments, the multireporter cell comprises a multicistronic reporter vector which comprises a promoter operably linked to an open reading frame, wherein the open reading frame comprises a first cistron, a second cistron, a third cistron and a fourth cistron, wherein each cistron comprises 5′ to 3′ nucleic acid encoding a transgene product fused to a fluorescent reporter polypeptide and nucleic acid encoding a linker peptide; wherein the first cistron and the second cistron are separated by nucleic acid encoding a first viral cleavage peptide, the second cistron and the third cistron are separated by nucleic acid encoding a second viral cleavage peptide the third cistron and the fourth cistron are separated by nucleic acid encoding an IRES. In some embodiments, the multireporter cell comprises a mutlticistronic reporter vector further comprises one or more inducible elements located between the promoter and the open reading frame.

In some embodiments, the inducible element of the multireporter cell is a Tet operator 2 (TetO2) inducible element.

In some embodiments, the promoter of the multireporter cell is a constitutive promoter. In some embodiments, the constitutive promoter is a CMV promoter, a TK promoter, an eF1-alpha promoter, a UbC promoter, a PGK promoter, a CAG promoter, an SV40 promoter, or a human β-actin promoter. In some embodiments, the promoter of the multireporter cell is a tissue specific promoter. In some embodiments, the tissue specific promoter is specific for cells of heart, blood, muscle, lung, liver, kidney, pancreas, brain, or skin. In some embodiments, the promoter of the multireporter cell is an inducible promoter. In some embodiments, the inducible promoter is a TRE promoter.

In some embodiments, the multireporter cell encodes one or more transgene products, wherein the one or more transgene products comprise polypeptides that can be used to profile a single biological pathway, cross-talk between two or more biological pathways, cellular homeostasis, organelle homeostasis, a toxicity response or phenotypic features. In some embodiments, the profile is performed on a single cell. In some embodiments, the reporter polypeptide can be visualized by microscopy, high throughput microscopy, fluorescence-activated cell sorting (FACS), lumininesence, or using a plate reader.

In some embodiments, the multireporter cell further comprising one, two or three transcription units comprising a promoter and nucleic acid encoding a transgene located 5′ to the open reading frame comprising two or more cistrons, wherein the reporter vector further comprises a core insulator sequence located 3′ to the transcription units and 5′ to the open reading frame comprising two or more cistrons.

In some embodiments, the invention provides a method for generating a multireporter cell comprising introducing any of the multicistronic reporter vector described herein into any of the acceptor cells described herein. In some embodiments, the recombinase associated nucleic acid sequence is a FRT nucleic acid sequence and the acceptor cell comprises a flp recombinase. In some embodiments, the recombinase associated nucleic acid is attP and the acceptor cell comprises a Bxb1 recombinase, a PhiC31 recombinase, or R4 recombinase. In some embodiments, the recombinase associated nucleic acid sequence is loxP nucleic acid sequence and the acceptor cell comprises a CRE recombinase.

In some embodiments, the invention provides a library of multireporter vectors, wherein the library comprises multicistronic reporter vectors comprising different transgenes encoding polypeptides fused to reporter polypeptides or a plurality of reporter cells as described herein, wherein two or more of the different transgenes on each vector are expressed essentially stoichiometrically when introduced to cells. In some embodiments, the library comprises reporter vectors that encode one or more transgenes one or more polypeptides comprise polypeptides that can be used to profile a single biological pathway, cross-talk between two or more biological pathways, cellular homeostasis, organelle homeostasis, a toxicity response or phenotypic features. In some embodiments, the biological pathway is a pathway associated with a disease. In some embodiments, the disease is cancer, a cardiovascular disease, a neurodegenerative disease or an autoimmune disease. In some embodiments, the biological pathway is a pathway associated with toxic response mechanism within the cell. In some embodiments, the biological pathway is a pathway associated with cell proliferation, cell differentiation, cell death, apoptosis, autophagy, DNA damage and repair, oxidative stress, chromatin/epigenetics, MAPK signaling, PI3K/Akt signaling, translational control, cell cycle and checkpoint control, cellular metabolism, development and differentiation signaling, immunology and inflammation signaling, tyrosine kinase signaling, vesicle trafficking, cytoskeletal regulation or ubiquitin pathway. In some embodiments, the library comprises reporter cells that are isogenic; i.e., the reporter cells are made from a single acceptor cell line by introducing multiple different reporter vectors thereby generating multiple different reporter cells based on the single acceptor cell line.

In some embodiments, each multicistronic vector of the library comprises transgenes used to profile a specific single biological pathway, specific cross-talk between two or more biological pathways, synthetic lethality, a specific cellular homeostasis, a specific organelle homeostasis, a specific toxicity response, a specific tissue, or phenotypic features, comprises a common transgene encoding a polypeptide fused to a different reporter polypeptide for each multicistronic reporter vector. In some embodiments, each multicistronic vector of the library comprising transgenes used to profile a specific single biological pathway, specific cross-talk between two or more biological pathways, a specific cellular homeostasis, a specific organelle homeostasis, a specific toxicity response, or phenotypic features comprises a common transgene encoding a polypeptide fused to reporter polypeptide.

In some embodiments, the invention provides a library of acceptor cells for receiving multicistronic reporter vectors, wherein the library comprises any of the acceptor cells described herein. In some embodiments, each cell in the library comprises multicistronic reporter vector comprising different transgenes encoding polypeptides fused to reporter polypeptides, wherein the different transgenes on each vector are expressed essentially stoichiometrically when introduced to cells.

In some embodiments, the library comprises different immortalized cells. In some embodiments, the library includes one or more of a HEK293T cell, an A549 cell, an U2OS cell, an RPE cell, an NPC1 cell, a MCF7 cell, a HepG2 cell, a HaCat cell, a TK6 cell, an A375 cell or a HeLa cell. In some embodiments, the library comprises different pluripotent, multipotent and/or progenitor cells. In some embodiments, the different pluripotent or multipotent cells include one or more of an induced pluripotent stem cell, a multipotent cell, a hematopoietic cell, an endothelial progenitor acceptor cell, a mesenchymal progenitor cell, a neural progenitor cell, an osteochondral progenitor cell, a lymphoid progenitor cell or a pancreatic progenitor cell. In some embodiments, the library of pluripotent or multipotent cells multireporter cells are differentiated after introduction of the multicistronic reporter vector. In some embodiments, the library comprises different primary cells. In some embodiments, the primary cells comprise one or more of a cardiomyocyte, a muscle cell, a lung cell, a liver cell, a kidney cell, a pancreatic cell, a neuron, or a tumor cell. In some embodiments, each cell in the library comprises the same multicistronic reporter vector. In some embodiments, cells in the library comprise different multicistronic reporter vectors. In some embodiments, the different multicistronic reporter vectors were introduced to isogenic acceptor cells.

In some embodiments, the invention provides a library of cells described herein, wherein the reporter vectors encode one or more transgenes one or more polypeptides comprise polypeptides that can be used to profile a single biological pathway, cross-talk between two or more biological pathways, cellular homeostasis, or phenotypic features. In some embodiments, the biological pathway is a pathway associated with a disease. In some embodiments, the disease is cancer, a cardiovascular disease, a neurodegenerative disease or an autoimmune disease. In some embodiments, the biological pathway is a pathway associated with toxic response mechanism within the cell. In some embodiments, the biological pathway is a pathway associated with cell proliferation, cell differentiation, cell death, apoptosis, autophagy, DNA damage and repair, oxidative stress, chromatin/epigenetics, MAPK signaling, PI3K/Akt signaling, translational control, cell cycle and checkpoint control, cellular metabolism, development and differentiation signaling, immunology and inflammation signaling, tyrosine kinase signaling, vesicle trafficking, cytoskeletal regulation or ubiquitin pathway.

In some embodiments, each multicistronic vector of the cells of the library comprises transgenes used to profile a specific single biological pathway, specific cross-talk between two or more biological pathways, synthetic lethality, a specific cellular homeostasis, a specific organelle homeostasis, a specific toxicity response, a specific tissue, and/or phenotypic features comprises a common transgene encoding a polypeptide fused to a different reporter polypeptide. In some embodiments, each multicistronic vector comprising transgenes used to profile a specific single biological pathway, specific cross-talk between two or more biological pathways, a specific cellular homeostasis, a specific organelle homeostasis, a specific toxicity response, and/or phenotypic features comprises a common transgene encoding a polypeptide fused to reporter polypeptide.

In some aspects, the invention provides kits comprising one or more multicistronic reporter vectors described herein. In some embodiments, the invention provides kits comprising one or more acceptor cells described herein. In some embodiments, the kit comprises one or more multicistronic reporter vectors described herein and one or more acceptor cells described herein.

In some aspects, the invention provides kits comprising one or more multireporter cells described herein. In some embodiments, the invention provides a kit comprising a library of acceptor cells and/or reporter cells arrayed in a multiwell plate. In some embodiments, the cells in the multiwell plate are cryopreserved.

In some aspects, the invention provides methods of profiling two or more polypeptides in a live cell, the method comprising determining the expression of the two or more of the transgenes and/or location of two or more transgene products of any of the multireporter cell described herein. In some embodiments, the method is used to profile a single biological pathway, cross-talk between two or more biological pathways, synthetic lethality, cellular homeostasis, organelle homeostasis, a toxicity response and/or phenotypic features. In some embodiments, the expression of the two or more of the transgenes and/or location of the two or more transgene products is determined at one or more time points. In some embodiments, the expression of the two or more of the transgenes and/or location of the two or more transgene products is determined at one or more of 1 minute, 5 minutes, 10 minutes, 15 minutes, 30 minutes, 45 minutes, 1 hour, 2 hours, 3 hours, 4 hours, 5 hours, 6 hours, 12 hours, 24 hours, 2 days, 4 days, 7 days, 14 days, 21 days, 30 days, 1 month, 3 month, 6 month, 9 month, 1 year, or anytime there between or more than 1 year after the initiation of the analysis.

In some embodiments, the invention provides methods of measuring the effects of an agent on the profile of two or more polypeptides in a live cell, the method comprising subjecting (i. e, contacting) a multireporter cell as described herein to the agent and determining the expression of the two or more of the transgenes and/or location of the two or more transgene products in the cell in response to the agent. In some embodiments, the agent is a drug or drug candidate. In some embodiments, the agent is a cancer drug or cancer drug agent. In some embodiments, the method is a toxicology screen.

In some embodiments, the invention provides methods for determining the expression of the two or more of the transgenes and/or location of the two or more transgene products is performed in a library of multireporter cells. In some embodiments, the profile is obtained using a single cell.

In some embodiments of the above methods, the expression of the two or more of the transgenes and/or location of the two or more transgene products expression and/or location of the two or more transgenes is measured by microscopy, high throughput microscopy, fluorescence-activated cell sorting (FACS), luminescence, using a plate reader, mass spectrometry, or deep sequencing.

### BRIEF DESCRIPTION OF THE DRAWINGS

FIGS. 1A-1C depict the generation of an acceptor cell line. FIG. 1A depicts the AAVS1 locus with acceptor site integration site (IS) and associated acceptor site design. The scheme shows junction PCR primers (bold half arrows) and Southern blot probes localization. The internal probe detects random integration while the 5′ probe allows for distinguishing of single or double allele integration. FIG. 1B depicts PCR primers (half arrows) that were designed to amplify the homologous recombination junction and were used to verify specific and successful integration. Example of junction PCR of single clones. * indicates positive clones. FIG. 1C depicts Southern blot analysis of candidate clones. EcoRI-digested genomic DNA was hybridized with 5′ probe (* indicate clones with single allele integration) (probe in FIG. 1A); arrows indicate the locations of the two AAVS1-hybridizing fragments derived from one targeted allele integration (TI). FIG. 1D depicts clones with single allele integration were further probed with internal probe (FIG. 1A) to verify single integration in the genome (arrow) (* indicates unspecific bands). FIG. 1E shows the first vs. a second acceptor site design. Cell lines were originally engineered with a single copy acceptor site for recombination containing: an SV40 constitutive promoter, a flippase recombination target (FRT) site, mCherry fluorescence marker, and Zeocin resistance gene. The second acceptor site design contains: an SV40 constitutve promoter, a flippase recombination target (FRT) site, an attP site to access recombination by serine recombinases, mCherry fluorescence marker, Zeocin resistance gene (Zeocin) or blasticidin resistance gene (blasticidin-S deaminase, Bsd), and CMV-GFP located after the AAVS1-Right homology arm (AAVS1-R). CMV-GFP in the redesigned acceptor site plasmid allows for differentiation between random and targeted integrations. Cells with random integration fluoresce green due to GFP expression, while cells with targeted integration do not fluoresce green due to loss of CMV-GFP since it's outside the acceptor site region integrated into cells (between AAVS1-L and AAVS1-R). FIG. IF shows that the new acceptor site enables FACS-based single cell sorting. Example of FACS data of wildtype Hek293 cells (left panel) and cells after integration of the acceptor site (right panel). The dual color (mCherry and GFP) allows for selection of cells with at least one integration at the AAVS1 locus and without random integration (mCherry+/GFP-).

FIG. 2 shows the design of a multicistronic platform harbored into a comparable pFIT vector backbone. The customized design allows for expression of four polypeptides from the same ORF, under the same promoter and with recombination (FRT site) capability. Recombination into acceptor cell lines results in similar protein expression levels at a balanced level among all cells.

FIG. 3 is a diagram showing that Ras/MAPK signaling proteins localize in different cellular compartments, depending on pathway activation status.

FIG. 4 shows a scheme delineating 1) incorporation of an acceptor site into the safe harbor locus of the target cell line; 2) the multicistronic MAPK reporter vector; and 3) recombination of the MAPK reporter vector into the acceptor site of the acceptor cell line to generate the reporter cell line.

FIG. 5 depicts the use of the HEK293TA+4-MAPK reporter cell line to track MAPK expression. Engineered multireporter cell line was induced for MAPK reporter expression and cells were treated with GDC-0879 (Raf inhibitor), FR 180204 (Erk inhibitor), Lovastatin (KRas inhibitor) and PD0325901 (Mek inhibitor) for 4 or 9h. Cells were imaged in a widefield microscope (scale bar 10 μm).

FIG. 6A shows a summary of the library of 24 MAPK multicistronic vectors carrying 4 proteins from the MAPK pathway. mCherry-Erk, Venus-Ras and mCerulean-Raf are fluorescently labeled while Mek is untagged. mCherry-Erk and untagged Mek are fixed for all vectors while Ras and Raf have different isoforms and mutants that are commonly found in cancer cells. MAPK constructs are denoted “MAPK R” and numbered from “1-24”. FIG. 6B shows twenty-three MAPK reporter cell lines were generated by engineering U2OSA cells with multireporter vectors and Bxb1. All reporter cell lines are under Tetracycline induction. Cells were imaged with an epifluorescence Nikon™ microscope with an OKOLab™ incubator system and images were acquired using a Plan Fluor™ 40× objective and NIS-Elements® software (Nikon™).

FIGS. 7A-7C show that U2OS^{A-Tet} MAPK R19, R20 and R21 reporter cell lines are sensitive to Trametinib and PD 0325901 inhibition. FIG. 7A shows a representative example of cells expressing K-Ras(wt), K-Ras(G12C) and K-Ras(G12D) exposed to Trametinib and PD 0325901 for 1 hr show an increase of mCherry-Erk at the cytoplasm as compared to DMSO treated cells. FIG. 7B shows the results of inhibitors that were tested as single agents in four-point ten-fold serial dilutions in duplicate starting at 10 μM (Trametinib (black), PD 0325901(grey). The percentage of cells with activated MAPK signaling for each sample was calculated and is plotted as mean with standard deviation. Dotted lines represent minimum and maximum values in the experiment. The subcellular localization of mCherry tagged ERK was used as a metric for MAPK pathway activation; cells with active MAPK signaling show with nuclear localization of ERK whereas cells with inactive or inhibited MAPK signaling show nuclear exclusion of ERK. MAPK pathway inhibitors were tested for their ability to inhibit nuclear localization of mCherry-tagged ERK in three different cell lines expressing K-Ras wild type, K-Ras G12C or K-Ras G12D. To quantify these images we calculated the ratio of nuclear to cytoplasmic ERK in single cells for four images per sample and plotted the resulting values as a histogram. FIG. 7C shows the mean IC₅₀ values from replicate experiments were calculated for the two inhibitors in each of the three cell lines.

FIGS. 8A-8C depict an example of the QC steps performed for U2OS^{A-Tet} acceptor cell line clone 23. FIG. 8A shows genomic DNA sequencing results of the acceptor site integration in the AAVS1 locus, the sequence convers the 5′ and 3′ flanking regions of the integration showing that no mutations/deletions or insertions occurred. FIG. 8B shows 2D plots and histograms mCherry fluorescence levels in U2OS(wt) cells and U2OS^{A-Tet} acceptor cells show that more than 99% of cells in the U2OS^{A-Tet} cells are mCherry(+). FIG. 8C shows 2D-fluorescence density plots of the FACS analysis conducted on U2OS^{A-Tet} MTS-Venus:H2B-TagBFP TagBFP reporter cells. Non-induced reporter cells are mCherry negative demonstrating the specificity of the integration events into the acceptor site. Same reporter cells after induction with doxycycline show significant uniformity in reporter expression levels within the polyclonal population of cells after integration of the multicistronic construct. Coefficient of correlation (r) was calculated from Venus and TagBFP fluorescence levels and indicates the linear relationship between the expression of the different tagged proteins.

FIG. 9 depicts exemplary multicistronic and universal platforms that can be used. The multicistronic and universal platform constructs were designed to include exemplary constitutive promoters including a cytomegalovirus immediate-early promoter (CMV), human thymidine kinase promoter (TK), and chicken β-Actin promoter coupled with CMV early enhancer (CAG). The reporter vectors were also designed to contain genes encoding exemplary resistance markers including a Hygromycin resistance gene (Hygro), Zeocin resistance gene (Zeo), and Puromycin resistance gene (Puro).

FIGS. 10A-10C depict the characterization of U2OS^{A} 3-Tox^{ORG} reporter cell line. FIG. 10A shows engineered multireporter cell lines were imaged in a widefield microscope (scale bar 10 μm). FIGS. 10B and 10C show engineered multireporter cell lines were analyzed using a flow cytometry analysis (FACS). FIG. 10B shows histograms of the 3 fluorescent reporters expressed by U2OSAToxORG cells showing similar coefficient of variations (CV) for all the 3 fluorescently labeled proteins and FIG. 10C shows a 2D-Density plot showing that the expression of both MTS-Venus and H2B-TagBFP reporters is highly correlated (r=87%).

FIGS. 11A and 11B show new U2OS^{A} Tox^{DUPR} reporter cell line report on DNA damage inducer agents. FIG. 11A is a representative example of U2OS Tox^{DUPR} stable cell line expressing 53BP1-mCerulean, XBP1-Venus and H2B-mCherry (not shown) exposed to vehicle, etoposide, Neocarzinostatin (NCS), aphidicolin, thapsigargin and tunicamycin for 8 hr. Only cells exposed to etoposide and NCS show an increase in the number of foci per nuclei. All the other compounds show number of foci similar to DMSO control. FIG. 11B shows results of compounds tested in four-point ten-fold serial dilutions in tetraplicates. The number of foci/nuclei was scored for each sample and is plotted as mean with standard deviation. The mean EC₅₀ values are indicated in the graph legend.

FIG. 12A-12E depict assay metrics for 53BP1 reporter from U2OSTox^{DUPR} cells. U2OSTox^{DUPR} reporter cells were plated in a 384 well plate treated either with vehicle (DMSO) or 1 μM etoposide and 500 ng/μl NCS—DNA damage inducers that increase the number of 53BP1 foci. For each condition there were 48 wells across the plate to determine spatial uniformity. Images were processed using CellProfiler (open source software for image analysis, Kamensty, L, et al. Bioinformatics (2011)) to count the number of foci/nuclei. Data was further computed using python programming language (Python Software Foundation, world wide web at python.org) and plots were generated using GraphPad Prism 7.0 Software (La Jolla, Calif., USA). FIGS. 12A and 12B show that no drift or edge effects are observed. Scatter plot of the response (number of 53BP1 Foci/nuclei) is plotted against well number, where the wells are ordered either by (FIG. 12A) row first, then by column, or by (FIG. 12B) column first, then by row. FIGS. 12C-12E show scatter plots of the mean number of foci/nuclei in U2OSTox^{DUPR} cells after treatment with Etoposide (FIG. 12C) or NCS (FIG. 12D) and compared to vehicle. Thick lines represent mean values and dashed lines represent 3× standard deviations. FIG. 12E provides a summary of Mean, StDev and Z′ for each compound. Z′ was calculated using the following formula: Z\hat{ }′=1-((3*StDev(Cpd)+3*StDev(DMSO)))/((Average(Cpd)-Average(DMSO)).

FIG. 13 shows the use of U2OS^{A} Tox^{ORG} and Tox^{DUPR} reporter cell lines as single reporters or pooled in the same well to evaluate toxicity. Top panel shows representative images of U2OS^{A} Tox^{ORG} or Tox^{DUPR} and after mixing both cell lines with a 1:1 ratio. Cells were plated 24h before image acquisition. The bottom panel is a table depicting the fluorescently labeled reporters contained in each of the Tox reporters. Note that the DNA marker (H2B) is common to both reporters however it is labeled with two different fluorophores—this enables the separation of both reporter cell lines during image analysis.

FIG. 14 shows the use of U2OSA Tox^{ORG} and Tox^{DUPR} reporter cell lines pooled in the same well to evaluate toxicity. Top panel shows a representative example of mixed U2OS Tox^{ORG} and U2OS Tox^{DUPR} in the same well. Cells were exposed to vehicle and Neocarzinostatin (NCS) for 8 hr. Only cells that express the of U2OSA Tox^{DUPR} and are exposed to NCS show an increase in the number of foci per nuclei. Scale bar 10 μm. Bottom panels shows results of testing NCS in four-point ten-fold serial dilutions in duplicates and tetraplicates for the highest concentration. The number of foci/nuclei was scored for each sample and is plotted as mean with standard deviation. The mean EC₅₀ values is indicated in the graph legend.

FIG. 15 depicts the expression of a 4-color multireporter transiently expressed in U2OS cells. A multicistronic reporter containing H2B-TagBFP, mCherry-LC3, MTS-Venus and palm-miRFP was transiently transfected in U2OS cells. 48h after transfection cells were plated in a glass bottom plate and imaged 24 hours later.

FIGS. 16A and 16B shows the successful generation of an iPS acceptor cell line for reporter recombination. FIG. 16A shows the acceptor site design. FIG. 16B shows NCRM5 iPSC colonies with correct integration of the acceptor site (mCherry positive and GFP negative).

FIGS. 17A-17C depict the process of engineering an iPS acceptor cell line, designing a 4-TOX reporter construct and cloning such construct into the acceptor line. Specifically, FIG. 17A shows the design of the targeting vector to engineer an acceptor cell line with the ‘landing pad’ in the AAVS1 locus using Cripsr-Cas9 directed sgRNAs. Note: mCherry is expressed under the SV40 promoter and ATG-Frt initiation site. FIG. 17B shows the design of the 4-TOX reporter: tetracistronic expression construct incorporating 4 multiple cloning sites (MCS) separated by 3 unique tandem viral 2A cleavage peptides inserts under the aMHC promoter (or CAG promoter) and an IRES element, followed by one Frt. FIG. 17C shows how once recombination occurs and insertion of 4-color reporter into the genome at the FRT site brings the SV40 promoter and the ATG initiation codon into proximity and in frame with the hygromycin resistance gene, and inactivates the mCherry gene.

FIG. 18 depicts schemes for the generation of alternative reporter cell lines. A multireporter line was developed to incorporate plug and play components (indicated by black boxes) and testable elements (indicated by grey boxes). The plasmid includes several modular elements that were previously validated (indicated by dashed boxes). As shown in FIG. 18, the reporter constructs were designed to include exemplary constitutive promoters including a cytomegalovirus immediate-early promoter (CMV), human elongation factor 1a promoter (EF1α), human Ubiquitin C promoter (Ubc), and chicken β-Actin promoter coupled with CMV early enhancer (CAG). The reporter vectors were also designed to contain genes encoding exemplary resistance markers including a Hygromycin resistance gene (Hygro), Zeocin™ resistance gene (Zeo), Neomycin resistance gene (Neo), and Puromycin resistance gene (Puro).

### DETAILED DESCRIPTION OF THE INVENTION

The present invention provides multiplex high content assays that can profile multiple polypeptides in live cells (e.g., in single live cells). In some aspects, the invention provides a multicistronic reporter vector comprising: a promoter operably linked to an open reading frame, wherein the open reading frame comprises two or more cistrons, and wherein expression of the open reading frame in a cell yields separate component polypeptide products from each cistron; wherein each cistron comprises a multiple cloning site (MCS) and nucleic acid encoding a reporter vector, wherein each cistron encodes a different reporter polypeptide; and wherein expression of two or more nucleic acids encoding polypeptides inserted into the two or more multiple cloning sites and fused to the reporter polypeptides is essentially stoichiometric. In some aspects, the invention provides acceptor cells for receiving the multicistronic reporter vectors. In yet other aspects, the invention provides multireporter cells for multiplex high content assays wherein the multireporter cells comprise any of the multicistronic reporter vectors described herein. The multireporter cells described herein may be used in live cell assays to profile the expression and activity of multiple polypeptides in live cells (e.g., single live cells) as a means to profile aspects of cell behavior including, but not limited to, biological pathways, cross-talk between biological pathways, cellular homeostasis, organelle homeostasis and toxicity and perturbations to these behaviors that may be induced by a candidate therapeutic or other chemical compound or other stimuli or combinations thereof.

Live-cell screening (e.g., LC-HCS) offers the opportunity to screen compounds in cellular systems that recapitulate the dynamic nature of signal transduction and cellular phenotypes that is not captured by end point assays. Immortalized cell lines can be used to monitor toxicity of various compounds as well as determine the effects that molecules, such as therapeutic candidate compounds of interest can have on specific pathways. The ability to easily maintain and work with these cells allows them to be a powerful tool in cell and molecular biology. Stem cells or human induced pluripotent stem cells (iPSCs) have great potential as cellular models used in live cell screening by providing physiological relevance and high reproductivity.

Described herein are novel methods, cells and multiplexed high-throughput assays that provide mechanistic and phenotypic readouts of cellular stress, homeostasis, and related events in immortalized, primary and human iPS cells, which have the potential of being differentiated into a variety of cell types in vitro. A wide variety of chemicals are known to perturb homeostasis and cause cellular stress, and it is thus an important aspect of cellular physiology to monitor in the context of understanding mechanisms of activity or toxicity mechanisms of candidate therapeutics. The methods described herein may be used to interrogate any potential collateral cytotoxicity of therapeutic agents, particularly anti-cancer drugs.

In some embodiments, the methods, cells and multiplexed high-throughput assays are used to profile cardiotoxicity. Cardiotoxicity represents a detrimental side effect of cancer treatment, resulting in considerable morbidity and mortality. Cytotoxic agents and targeted therapies used to treat cancer, including classic chemotherapeutic agents, antibodies and small molecule tyrosine kinase inhibitors, and chemoprevention agents all affect the cardiovascular system and may result in severe effects such as heart failure, ventricular dysfunction, and myocardial ischemia. The rise in cancer therapy-induced cardiomyopathies suggests that the risks of cardiotoxicity must be carefully weighed during the evaluation and development of any anti-cancer drug.

The molecular mechanisms linking cancer therapies to cardiomyopathies, including the specific contribution of stress-induced transcription factors to cell survival or death, are not well understood due to the lack of a system for real-time monitoring of this aspect of toxicology in biologically-relevant cardiac cells. The assays described herein address this need through provision of multiplexed fluorescent reporter systems that provide readout of cellular stress and organelle homeostasis using human iPSC-derived cardiomyocytes. The effects of molecules and potential therapeutic agents can be assessed for neuro toxicity, hepatic toxicity, or any other type of tissue toxicity.

The major limitations of using primary cardiomyocytes, primary neurons or other primary cell types, are the technical difficulties associated with obtaining and maintaining these cells. For example while immortalized cardiac cells are convenient because they can readily proliferate, beat, and in some cases, stably maintain a cardiac phenotype, their metabolism and morphology may be different from cardiomyocytes so their use has been limited in toxicology studies. Cardiomyocytes derived from stem cells or iPSCs overcome these disadvantages and provide a tool to not only assess the effect of molecules on the terminally differentiated cells, but also to study development or the effect of various molecules through different stages of differentiation. This is particularly important since reliable tests on progenitor and differentiating cells is valuable but sparse. Furthermore, iPSCs can be generated from human subjects to examine a variety of diseased and normal phenotypes.

In some embodiments, the methods, cells and multiplexed high-throughput assays are used in drug discovery. For example, the methods, cells and multiplexed high-throughput assays are used in drug discovery to treat neurodegeneration. In some embodiments, the invention provides the use of iPSC-derived cells in drug development to treat neurodegenerative diseases.

Definitions

A “vector,” as used herein, refers to a recombinant plasmid or virus that comprises a nucleic acid to be delivered into a host cell, either in vitro or in vivo.

The term “polynucleotide” or “nucleic acid” as used herein refers to a polymeric form of nucleotides of any length, either ribonucleotides or deoxyribonucleotides. Thus, this term includes, but is not limited to, single-, double- or multi-stranded DNA or RNA, genomic DNA, cDNA, DNA-RNA hybrids, or a polymer comprising purine and pyrimidine bases, or other natural, chemically or biochemically modified, non-natural, or derivatized nucleotide bases. The backbone of the nucleic acid can comprise sugars and phosphate groups (as may typically be found in RNA or DNA), or modified or substituted sugar or phosphate groups. Alternatively, the backbone of the nucleic acid can comprise a polymer of synthetic subunits such as phosphoramidates and thus can be an oligodeoxynucleoside phosphoramidate (P-NH2) or a mixed phosphoramidate- phosphodiester oligomer. In addition, a double-stranded nucleic acid can be obtained from the single stranded polynucleotide product of chemical synthesis either by synthesizing the complementary strand and annealing the strands under appropriate conditions, or by synthesizing the complementary strand de novo using a DNA polymerase with an appropriate primer.

The terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-translational modifications of the polypeptide, for example, glycosylation, sialylation, acetylation, phosphorylation, and the like. Furthermore, for purposes of the present invention, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

A “biosensor” as used herein, refers to reporter compounds that are attached to an additional protein sequence that make it sensitive to small biomolecules or other physiological intracellular processes. In nonlimiting examples, the biosensor is a fluorescent biosensor including a genetically encoded fluorescent polypeptide. Biosensors are introduced into cells, tissues or organisms to allow for detection (e.g., by fluorescence microscopy) as a difference in FRET efficiency, translocation of the fluorescent protein or modulation of the reporter properties of a single reporter protein. Many biosensors allow for long-term imaging and can be designed to specifically target cellular compartments or organelles. Another advantage of biosensors is that they permit investigation of a signaling pathway or measurement of a biomolecule while largely preserving spatial and temporal cellular processes.

An “acceptor cell” is a cell which has been engineered to harbor an acceptor construct in its genome.

An “acceptor construct” is sequence of nucleotide which comprises a sequence of nucleic acid which can harbor a reporter nucleic acid.

The term “transgene” refers to a nucleic acid that is introduced into a cell and is capable of being transcribed into RNA and optionally, translated and/or expressed under appropriate conditions.

As used herein “stem cell”, unless defined further, refers to any non-somatic cell. Any cell that is not a terminally differentiated or terminally committed cell may be referred to as a stem cell. This includes embryonic stem cells, induced pluripotent stem cells, progenitor cells, and partially differentiated progenitor cells. Any cell which has the potential to differentiate into two different types of cells is considered a stem cell for the purpose of this application.

An “iPS” cell as used herein refers to any pluripotent cell obtained by re-programing a non-pluripotent cell. The reprogrammed cell may have been generated by reprogramming a progenitor cell, a partially-differentiated cell, or a fully differentiated cell of any embryonic or extraembryonic tissue lineage.

“Reprogramming” as used herein refers to the process of de-differentiating a cell which is at least partially differentiated into a pluripotent state.

As used herein “immune privileged cell” refers to a cell which elicits a diminished immune response when introduced into a foreign host organism.

As used herein “cistron” refers to a segment of nucleic that is equivalent to a gene and that encodes a single functional unit (e.g., a single polypeptide or a fusion polypeptide comprising a transgene product and a reporter domain). As used herein, a multicistronic vector is a nucleic acid that comprises two or more cistrons. In some embodiments, the multicistronic vector comprises two or more cistrons in a single open reading frame. In some embodiments, the single open reading frame, when translated, generates two or more polypeptides that are dissociated from one another.

As used herein, the term “essentially stoichiometric” with regard to expression of two or more reporter polypeptides refers to the expression of two or more reporter polypeptides wherein the expression level of the two or more reporter polypeptides are equal or vary by no more than about 5%, 10%, 15%, 20% or 25% of each other.

As used herein, a “site-specific recombinase sequence” refers to a target sequence of site-specific recombination system. Site-specific recombination systems include, but are not limited to, Tyr recombinases, Ser integrases, Cre recombinases with loxP target sequences, FLP recombinase with FRT target sequence. Site-specific recombination nucleic acid sequences for Tyr recombinases, and Ser integrases (e.g., PhiC31) integrases include but are not limited to attB, and attP. Site-specific recombination nucleic acid sequences for CRE recombinase include but is not limited loxP. Site-specific recombination nucleic acid sequences for FLP recombinase include but is not limited FRT.

Reference to “about” a value or parameter herein includes (and describes) embodiments that are directed to that value or parameter per se. For example, description referring to “about X” includes description of “X.”

As used herein, the singular form of the articles “a,” “an,” and “the” includes plural references unless indicated otherwise.

It is understood that aspects and embodiments of the invention described herein include “comprising,” “consisting,” and/or “consisting essentially of” aspects and embodiments

Acceptor Cells

The present disclosure provides multireporter cells and methods for generating multireporter cells, which can be used to profile two or more polypeptides in a live cell. Multireporter cells are developed by cloning a multicistronic reporter vector into an insertion site of an acceptor cell. Acceptor cells are developed by incorporating recombinant nucleic acid encoding an acceptor sequence into the genome of a cell. The acceptor sequence comprises an insertion site which allows for the site-specific integration of the multicistonic reporter vector into the acceptor cell genome. As described herein, the multicistronic reporter vector comprises nucleic acid encoding two or more polypeptides wherein the polypeptides are fused to a reporter domain. The two or more nucleic acid sequences encoding for the polypeptides of interest are located within the same open reading frame, allowing for essentially stoichiometric expression of the recombinant peptides. In some embodiments, the essentially stoichiometric expression is expression of two or more reporter polypeptides wherein the expression level of the two or more reporter polypeptides is essentially the same; i.e., have 1:1 stoichiometry. In some embodiments, the expression level of the two or more reporter polypeptides are equal or vary by no more than any of about 5%, 10%, 15%, 20% or 25% of each other.. In some embodiments, the essentially stoichiometric expression is the stoichiometric expression of two, three, four or more than four reporter polypeptides. Expression levels of the two or more reporter polypeptides can be measured by any means known in the art; for example, by fluorescence detection, by immunoassay, by enzyme assay, by measuring RNA levels (e.g. qPCR), etc.

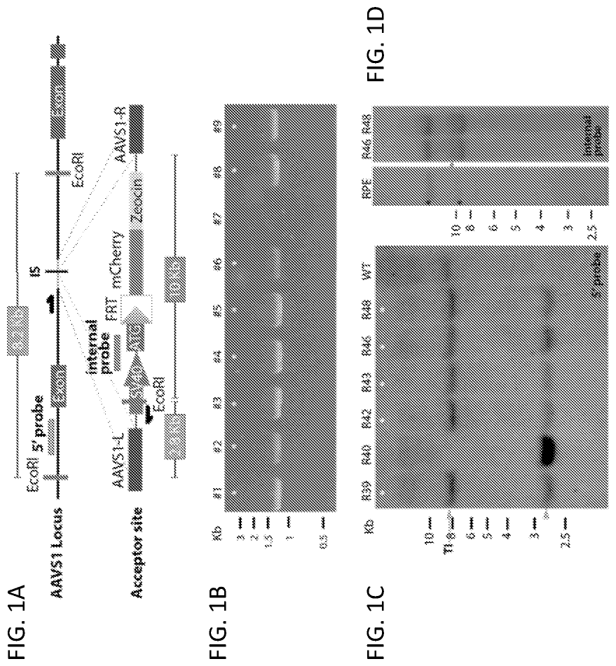

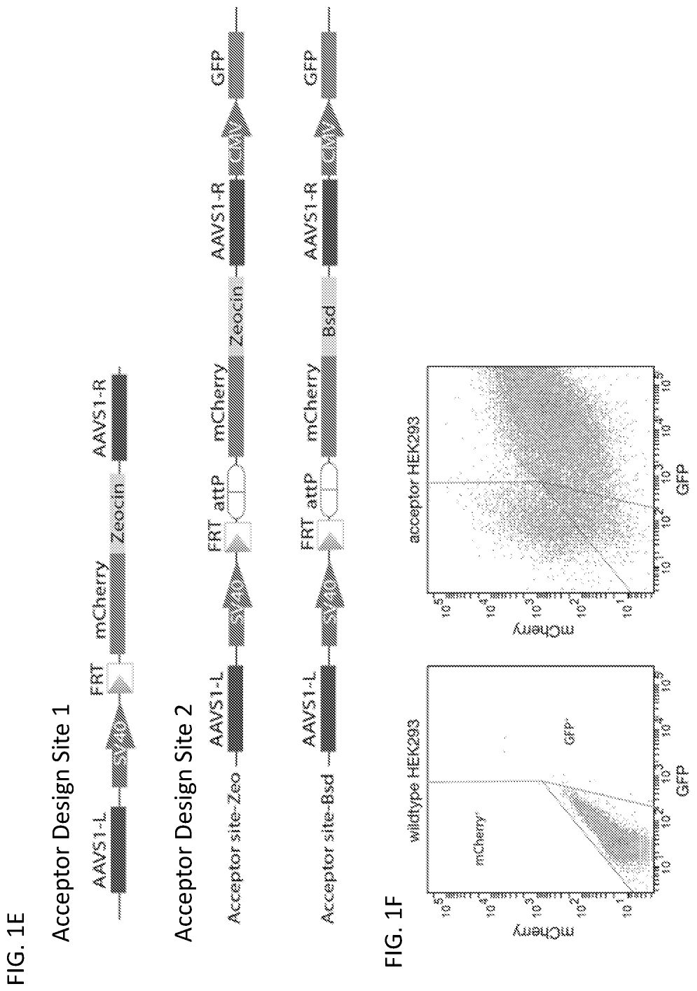

In some aspects, the invention provides an acceptor cell for receiving a multicistronic reporter vector, wherein the acceptor cell comprises a recombinant nucleic acid integrated into a specific site in a host cell genome, wherein the recombinant nucleic acid comprises a first promoter operably linked to nucleic acid encoding a fusion polypeptide, wherein the fusion polypeptide comprises a reporter domain and a selectable marker domain, and wherein the nucleic acid comprises a site-specific recombinase nucleic acid sequence located at the 5′ end of the nucleic acid encoding the fusion polypeptide. Nonlimiting examples of acceptor sites are provided in FIG. 1E.

In some embodiments, the promoter (e.g., the first promoter) is a constitutive promoter. Examples of constitutive promoters include but are not limited to a cytomegalovirus immediate early (CMV) promoter, a thymidine kinase (TK) promoter, an eF1-alpha, a Ubiquitin C (UbC), a Phosphoglycerate Kinase (PGK), a CAG promoter, an SV40 promoter, or a human β-actin promoter. In some embodiments, the promoter is an inducible promoter. Examples of inducible promoters include but are not limited to a tetracycline responsive promoter, a rapamycin-regulated promoter, and a sterol inducible promoter. In some embodiments, the inducible promoter is a tetracycline responsive promoter.

In some embodiments of the invention, the acceptor site comprises a site-specific recombinase sequence. Examples of site-specific recombinase sequences include but are not limited to a FRT nucleic acid sequence, an attP nucleic acid sequence and loxP nucleic acid sequence. In some embodiments, the site-specific recombinase sequence is a FRT nucleic acid. In some embodiments, the site-specific recombinase sequence is a attP nucleic acid. In some embodiments, the site-specific recombinase sequence is a attB nucleic acid. In some embodiments, the site-specific recombinase sequence is a loxP nucleic acid. In some embodiments, the site-specific recombinase sequence is a FRT nucleic acid and an attP sequence. In some embodiments, the site-specific recombinase sequence is a FRT nucleic acid and an attB sequence.

In some embodiments, the acceptor site comprises nucleic acid encoding a reporter polypeptide. In some embodiments, the acceptor site comprises nucleic acid encoding a selection polypeptide. In some embodiments, the acceptor site comprises nucleic acid encoding a reporter polypeptide (e.g., a reporter domain) fused to a selection polypeptide (e.g., a selection domain). In some embodiments, the reporter polypeptide is on the N-terminus of the fusion polypeptide and the selection polypeptide is on the C-terminus of the fusion polypeptide. In other embodiments, the selection polypeptide is on the N-terminus of the fusion polypeptide and the reporter polypeptide is on the C-terminus of the fusion polypeptide.

A reporter peptide is a peptide which can be readily identified; for example via microscopy, plate reader, FACS, chemically, mass spectometry, or deep sequencing. For example the reporter domain may be a fluorescent or luminescent polypeptides. In some embodiments, the reporter domain may be a green fluorescent protein (GFP) or any of its derivatives. In some embodiments the reporter domain is a non GFP derived fluorescent peptide. In some embodiments the reporter domain encodes for GFP, EGFP, Emerald, Citrine, Venus, mOrange, mCherry, TagBFP, mTurquoise, Cerulean, UnaG, dsRed, eqFP611, Dronpa, RFP, TagRFPs, TdTomato, KFP, EosFP, Dendra, IrisFP, iRFPs, or smURFP. The reporter domain may be a luciferase. The reporter domain may be an enzyme, which when expressed allows for visualization of expression through the products of a chemical reaction. In some embodiments, the reporter is a firefly luciferase or a renilla luciferase. In some embodiments the reporter domain is β-glucuronidase or β-galactosidase.

A selectable marker domain may be a polypeptide which confers resistance to a molecule the cell is not normally resistant to, or at a dose the cell is not normally resistant to. For example the selectable marker domain may be a polypeptide which confers resistance to an antibiotic. In some embodiments the selectable marker is polypeptide that confers resistance to blasticidin, geneticin, hygromycin, puromycin, neomycin, Zeocin™, kanamycin, carbenicillin, ampicillin, antinomycin, apramycin, mycophenolic acid, histidinol, methotrexate or any of their salts or derivatives.

In some embodiments, the acceptor cell comprises an acceptor site which comprises nucleic acid encoding a fusion polypeptide comprising a reporter domain and a selection domain. In some embodiments, the reporter domain of the fusion polypeptide is an mCherry reporter domain. In some embodiments, the selectable marker domain of the fusion polypeptide confers resistance to hygromycin, Zeocin™, puromycin, blasticidin, neomycin or an analog of hygromycin, Zeocin™, puromycin, blasticidin, neomycin.

In some embodiments, the acceptor site further comprises nucleic acid encoding a gene expression repressor polypeptide. In some embodiments, the acceptor site comprises nucleic acid encoding a tetracycline repressor polypeptide operably linked to a promoter. In some embodiments, the constitutive promoter is a CMV promoter, a TK promoter, an eF1-alpha, an UbC promoter, a PGK promoter, a CAG promoter, an SV40 promoter, or a human β-actin promoter. In some embodiments, the promoter is a human β-actin promoter or a CAG promoter.

The acceptor site is integrated at a specific site in the genome of the acceptor cell. In some embodiments, the specific site is an innocuous site in the acceptor cell genome. For example, insertion of a nucleic acid into the specific site has little impact on the functions of the acceptor cell. In some embodiments, the recombinant nucleic acid is integrated in an adeno-associated virus S1 (AAVS1) locus, a chemokine (CC motif) receptor 5 (CCR5) locus, a human ortholog of the mouse ROSA26 locus, a hippl l (H11) locus or the citrate lyase beta like gene locus (CLYBL). In some embodiments, the acceptor site comprises heterologous nucleic acid sequences that were used to target the recombinant nucleic acid encoding the site-specific recombinase nucleic acid sequence to the specific target locus in the acceptor cell genome. In some embodiments, the acceptor cell comprises nucleic acid for targeting to the AAVS1 locus, the CCR5 locus, the mouse ROSA26 locus or the human ortholog of the mouse ROSA26 locus, a hippll (H11) locus or the CLYBL locus.

The present disclosure provides methods to generate acceptor cell lines. The method includes engineering a cell so that the cell can harbor a reporter nucleic acid. Any cell can be an acceptor cell. In some embodiments the cell used is a prokaryotic cell. In some embodiments the cell used is a eukaryotic cell. In some embodiments the cell used is a plant cell. In some embodiments the cell used is a fungal cell. In some embodiments the cell used is a mammalian cell. In some embodiments the cell used is a human cell. In some embodiments the acceptor cell is generated by engineering an immortalized cell. In some embodiments, the immortalized cell is a HEK293T cell, an A549 cell, an U2OS cell, an RPE cell, an NPC1 cell, a MCF7 cell, a HepG2 cell, a HaCat cell, a TK6 cell, an A375 cell or a HeLa cell. In some embodiments the acceptor cell line is generated by engineering a primary cell. The primary cell may be harvested from a plant or an animal. In some embodiments the primary cell is harvested from a mammal. In some embodiments the primary cell is harvested from a human. In some embodiments the primary cell is harvested for a rodent. In some embodiments the cell used is a patient specific cell.

In some embodiments the acceptor cell is a stem cell. The stem cell may be a totipotent, a pluripotent or a multipotent stem cell. Any totipotent, pluripotent, multipotent or progenitor stem cell may be used to generate an acceptor cell line. The stem cell may be an animal cell. In some embodiments the stem cell is from a mammal. In some embodiments the stem cell is from a human. In some embodiments, the stem cell is a patient specific stem cell. IN some embodiments, the stem cell is an autologous stem cell. In some embodiments, the stem cell is an allogeneic stem cell. In some cases the stem cell is from a non-human primate, a dog or a rodent. The stem cell may be derived from the trophectoderm, the inner cell mass of a blastocyst, or a specific tissue. The stem cell may be an embryonic stem cell, an induced pluripotent stem cell or a progenitor stem cell. Any progenitor cell can be used to generate an acceptor cell line. For example the progenitor cell used may be a hematopoietic cell, as endothelial progenitor cell, a mesenchymal progenitor cell, a neural progenitor cell, an osteochondral progenitor cell, a lymphoid progenitor cell or a pancreatic progenitor cell.

In some embodiments, the acceptor cell is a bacterial cell, a plant cell, a fungus cell or an animal cell. In some embodiments the animal is an invertebrate. In some embodiments the acceptor cell is a cell from a member of the Drosophila melanogaster species. In some embodiments the acceptor cell is a cell from a member of the Caenorhabditis elegans species. In some embodiments the acceptor cell is a vertebrate animal cell. In some embodiments the acceptor cell is a mammalian cell. In some embodiments the acceptor cell is a human cell, a primate cell, a rodent cell, a feline cell, a canine cell, a bovine cell, a porcine cell or an ovine cell.

In some embodiments, the invention provides a method for generating an acceptor cell for receiving a multicistronic reporter vector, the method comprising introducing a recombinant nucleic acid to a cell wherein the recombinant nucleic acid comprising 5′ to 3′ a first nucleic acid for targeting homologous recombination to a specific site in the cell, a first promoter, site-specific recombinase nucleic acid, nucleic acid encoding a first reporter polypeptide and a selectable marker, and a second nucleic acid for targeting homologous recombination to a specific site in the cell. In some embodiments, the recombinant nucleic acid comprises any of the acceptor sites described above to generate any of the acceptor cells described above.

In some embodiments, the invention provides a method for generating an acceptor cell for receiving a multicistronic reporter vector, the method comprising introducing a recombinant nucleic acid to a cell wherein the recombinant nucleic acid comprising 5′ to 3′ a first nucleic acid for targeting homologous recombination to a specific site in the cell, a first promoter, site-specific recombinase nucleic acid, nucleic acid encoding a first reporter polypeptide and a selectable marker, a second nucleic acid for targeting homologous recombination to a specific site in the cell, a second promoter and nucleic acid encoding a second reporter polypeptide, wherein expression of the first reporter polypeptide without expression of the second reporter polypeptide indicates targeting integration of the recombinant nucleic acid to the specific site in the cellular genome and expression of the first and second reporter polypeptides indicates random integration in the cellular genome. In some embodiments, the recombinant nucleic acid comprises any of the acceptor sites described above to generate any of the acceptor cells described above.

In some embodiments, the acceptor site further comprises nucleic acid encoding a gene expression repressor polypeptide. In some embodiments, the acceptor site comprises nucleic acid encoding a tetracycline repressor polypeptide operably linked to a promoter. In some embodiments, the constitutive promoter is a CMV promoter, a TK promoter, an eF1-alpha, an UbC promoter, a PGK promoter, a CAG promoter, an SV40 promoter, or a human β-actin promoter. In some embodiments, the promoter is a human β-actin promoter or a CAG promoter.

In some embodiments, the acceptor cell is generated by introducing an acceptor site to the acceptor cell. In some embodiments, the acceptor site comprises 1) two AAVS1 sequences to direct integration of the acceptor site to the AAVS1 locus; 2) a fluorescent marker visible by microscopy (mCherry); 3) an antibiotic (Zeocin-Thermo Fisher) resistance selection marker ; 4) a FRT site used for site-specific recombination of the reporter construct by flippase (Flp) ; 5) a constitutive promoter, SV40, that drives Zeocin (Thermo Scientific™) resistance gene and mCherry expression, enabling antibiotic selection and fluorescence screening, respectively, to identify positive acceptor cell clones; 6) human b-actin promoter driving the Tetracycline Repressor proteins (TetR).

The acceptor cell is generated by engineering the genome of a cell to include an acceptor construct. There are several techniques known in the art, which can be used to engineer a cell into harboring an exogenous nucleic acid sequence. For example, the acceptor cell may be generated by inserting the acceptor construct into a cell via a viral transfection system. In some embodiments the retrovirus used is a lentivirus or an adenovirus. In some embodiments the acceptor construct may be a vector. The vector may be a viral vector. In some embodiments the vector is a viral vector, such as a lentiviral vector, a baculoviral vector, an adenoviral vector, or an adeno-associated viral (AAV) vectors. In some embodiments an AAV transfection system is used to deliver the acceptor construct into the acceptor cells. The AAV used can be modified and optimized depending on the cell type or locus used. For example AAV1, AAV2, AAVS or any combination thereof can be used.

The acceptor construct may be delivered by other methods known in the art. Many means of delivery are known (such as yeast systems, microvesicles, gene guns/means of attaching vectors to gold nanoparticles). In some embodiments the acceptor construct may be delivered via liposomes, nanoparticles, exosomes, microvesicles, or a gene-gun.

In some embodiments the acceptor construct is inserted into the genome of a cell by use of an RNA guided endonuclease system. In some embodiments a CRISPR system is used. In some embodiments the acceptor construct is inserted into the genome of the cell using RNA guided genome engineering via Cas9. However, any nuclease that works in an RNA guided genome engineering system works. Nucleases that can be used include Cas3, Cas8a, Cas5, Cas8b, Cas8C, CaslOd, Cse, Cse2, Csy1, Csy2, Csy3, GSU0054, Cas10, Csm2, Cmr5, Cas10, Csx11, Csx10, Csf1, Cas9, Cas4, Csn2, Cpf1, C2c1, C2c3, C2c2. The type of endonuclease used may be dependent on the cell to be engineered and the target locus for insertion.

In some embodiments the acceptor construct is inserted into the genome of a cell by using a TALEN or a Zinc Finger endonuclease (ZFN).

RNA guided genome engineering via Cas9 offers improvements over TALEN and ZFN approaches for cell line engineering. Using ZFN for example has some limitations. Firstly, the ZFN technique requires synthesis of new vectors and RNA for the specific DNA binding sites in each new genomic integration locus that is to be modified. These typically require expensive optimization thus cost and complexity limits the flexibility of applying these techniques to more than one or two loci. By contrast, the RNA-guided system uses a single protein (Cas9) that requires only a short RNA molecule to program it for site-specific DNA recognition. The Cas9-RNA complex is thus easier to make than analogous ZFN targeting proteins and the system is consequently more flexible. Cas9-RNA complexes also have lower toxicity in mammalian cells than TALENs and ZFNs. In addition to Cas9, other nucleases associated with RNA guided genome editing can be used. RNA guided genome engineering is known in the art.

The nucleic acid encoding the acceptor site may be inserted into any part of the cell genome where it is possible to insert an exogenous sequence of DNA without disrupting transcription of an endogenous gene. In some embodiments, the acceptor site is targeting to the AAVS1 locus, the CCR5 locus, the mouse ROSA26 locus or the human ortholog of the mouse ROSA26 locus, a H11 locus or the CLYBL locus. In some embodiments the construct is inserted in a location within the genome which is not epigenetically silenced. In some embodiments the acceptor construct is inserted into the AAV genomic locus of the host cell. In some embodiments, a single copy of the nucleic acid encoding the acceptor site is incorporated into the acceptor cell genome (e.g., on a single allele of the acceptor cell genome).

The nucleic acid encoding the acceptor site may comprise two nucleic acid sequences which allow for homologous recombination into the genome of a cell. In some embodiments, where the acceptor construct is integrated into the AAVS1 genomic locus of a cell, the acceptor construct comprises two AAVS1 sequences which allow for direct integration of the acceptor construct into the cell. The reporter construct may comprise one or more sequences which allow the construct to be integrated in a different genomic locus of a cell. The acceptor construct may be inserted into a locus within the genome of a cell via homologous recombination or any other way of genomic engineering known in the art. In some embodiments, the acceptor construct comprises two AAVS1 sequences which allow the acceptor construct to be directly integrated into the AAVS1 locus of a cell. In some embodiments, the acceptor construct comprises two CCRS sequences which allow the acceptor construct to be directly integrated into the CCRS locus of a cell. In some embodiments, the acceptor construct comprises two ROSA26 sequences which allow the acceptor construct to be directly integrated into the ROSA26 locus of a mouse cell. In some embodiments, the acceptor construct comprises two human orthologs of the mouse ROSA26 sequences which allow the acceptor construct to be directly integrated into the human ortholog of the mouse ROSA26 locus of a human cell. In some embodiments, the acceptor construct comprises two CLYBL sequences which allow the acceptor construct to be directly integrated into the CLYBL locus of a cell. In some embodiments, the acceptor construct comprises two H11 sequences which allow the acceptor construct to be directly integrated into the H11 locus of a cell.

In some embodiments, the library enables pooled screening of different reported cell lines. For example, two or more reporter cell lines can be developed from a single isogenic acceptor cell line where the two or more reporter cell lines can be distinguished; for example by differentially labeling the nuclei. The two or more reported cell lines can be mixed together in one single assay to provide results on multiple polypeptides in one or more pathways.

In some embodiments, up to at least five quality control steps may be implemented to select for acceptor cell lines that have an integration site intact and fully functional with best performances. In some embodiments, genomic DNA of the acceptor cell lines at 5′ and 3′ site of insertion in AAVS1 genomic locus is sequenced to confirm that the recombination process did not trigger any small insertion, deletion or mutation flanking the recombination site. In some embodiments, the homogeneity of the acceptor cells is evaluated to ensure that they come from a single cell - for that flow cytometry analysis is conducted and the coefficient of variation (CV) of the fluorophore is calculated. In some embodiments, it is determined that the acceptor cells contain a functional tetracycline regulation; for example, the cells are co-transfected with B×B1 and a dual color multicistronic construct that contains MTS-Venus, H2B-TagBFP under tetracycline regulation. In some embodiments, reporter cells are selected by antibiotic resistance. For example, the number of colonies formed is scored. After selection, the cells are treated with 1 μg/ml doxycycline for 12-15h. The next validation and QC steps are conducted using flow cytometry analysis. In some embodiments, induced cells are compared to uninduced cells to verify loss of mCherry and gain of Venus and TagBFP fluorescence. In some embodiments, the variability of the protein expression in the polyclonal population after reporter integration is determined. In some embodiments, the protein expression is similar to that of the acceptor cell line population. In some embodiments, the CV of the fluorescence intensity of each of the fluorophores present in the multicistronic vector is determined and compared to the parental CV. In some embodiments, the expression of the different proteins placed on the acceptor site is homogenous and that the payload is verified; for example, by determining the coefficient of correlation (r) based on the flow cytometry values of fluorescence intensity of the proteins expressed.

Multicistronic Reporter Vectors

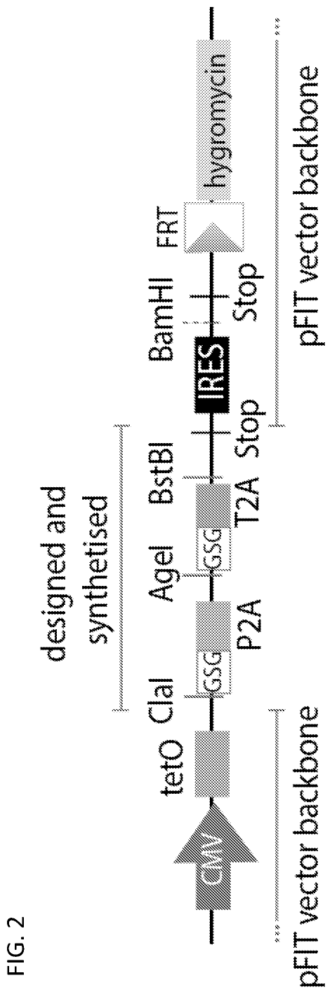

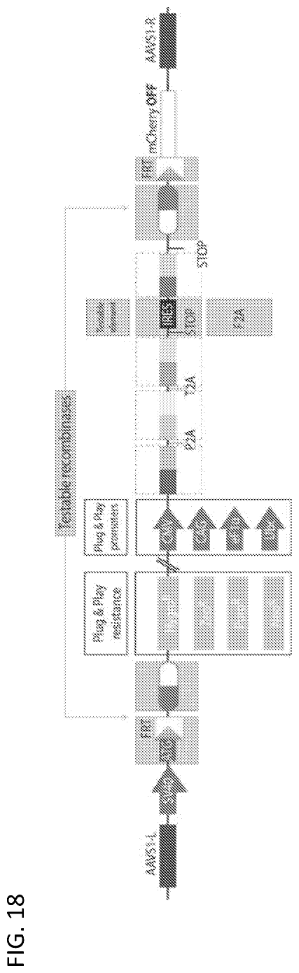

In some aspects, the invention provides multicistronic reporter vectors comprising: a promoter operably linked to an open reading frame, wherein the open reading frame comprises two or more cistrons, and wherein expression of the open reading frame in a cell yields separate component polypeptide products from each cistron; wherein each cistron comprises a multiple cloning site (MCS) and nucleic acid encoding a reporter vector, wherein each cistron encodes a different reporter polypeptide; and wherein expression of two or more nucleic acids encoding polypeptides inserted into the two or more multiple cloning sites and fused to the reporter polypeptides is essentially stoichiometric. The vector is designed for a “plug-and-play” mode wherein different promoters may be swapped in to drive expression of the open reading frame, different polypeptides of interest can be swapped in, different reporter polypeptide can be swapped in, and different selection polypeptides may be swapped in depending on the particular use of the multicistronic reporter vector. Likewise, the multicistronic reporter vector is designed, through the use of the various MCS sequence to insert nucleic acid encoding any polypeptide of interest, such that the transgene product, tagged to a reporter polypeptide, is expressed by the multicistronic reporter vector. In some embodiments, the multicistronic vector comprises the “backbone” vector wherein transgenes of interest have not been inserted into the MCS sequences. In other embodiments, the multicistronic vector includes vectors where transgenes of interest have been inserted into the MCS sequences such that expression of the open reading yields distinct reporter-tagged polypeptides. A nonlimiting example of a multicistronic reporter vector is provided in FIGS. 2 and 18.

In some embodiments, the essentially stoichiometric expression is expression of two or more polypeptides fused to reporter polypeptides wherein the expression level of the two or more polypeptides fused to reporter polypeptides is essentially the same; i.e., have 1:1 stoichiometry. In some embodiments, the expression level of the two or more reporter polypeptides are equal or vary by no more than any of about 5%, 10%, 15%, 20% or 25% of each other. In some embodiments, the essentially stoichiometric expression is the stoichiometric expression of two, three, four or more than four two or more polypeptides fused to reporter polypeptides. Expression levels of the two or more reporter polypeptides can be measured by any means known in the art; for example, by fluorescence detection, by immunoassay, by enzyme assay, by measuring RNA levels (e.g. qPCR), etc.

In some embodiments, the cistrons of the multicistronic reporter vector are separated from one another by nucleic acid encoding one or more self-cleaving peptide and/or one or more internal ribosome entry site (IRES). In some embodiments, the one or more self-cleaving peptides is a viral self-cleaving peptide. In some embodiments, the one or more viral self-cleaving peptides is one or more 2A peptides. In some embodiments, the one or more 2A peptides is a T2A peptide, a P2A peptide, an E2A peptide or a F2A peptide. In some embodiments, one or more of the cistrons of the open reading frame is separated from the other cistrons in the open reading frame by an IRES sequence. In some embodiments, the IRES is an encephalomyocarditis virus (EMCV) IRES, a Hepatitis C virus (HCV) IRES or an Enterovirus 71 (EV71) IRES.

In some embodiments, the multicistronic reporter vector comprises two cistrons wherein the two cistrons are separated by nucleic acid encoding a viral self-cleaving peptide or an IRES. In some embodiments, the multicistronic reporter vector comprises three cistrons wherein the three cistrons are separated by nucleic acid encoding viral self-cleaving peptides. In some embodiments, the multicistronic reporter vector comprises three cistrons wherein the three cistrons are separated by IRES sequences. In some embodiments, the multicistronic reporter vector comprises three cistrons wherein the first and second cistrons are separated by nucleic acid encoding a viral self-cleaving peptide and the second and third cistron is separated by an IRES sequence. In some embodiments, the multicistronic reporter vector comprises three cistrons wherein the first and second cistrons are separated by an IRES sequence and the second and third cistron is separated by nucleic acid encoding a self-cleaving peptide. In some embodiments, the multicistronic reporter vector comprises four cistrons wherein the cistrons are separated from one another by nucleic acid encoding viral self-cleaving peptides. In some embodiments, the multicistronic reporter vector comprises four cistrons wherein the cistrons are separated from one another by IRES sequences. In some embodiments, the multicistronic reporter vector comprises four cistrons wherein the first and second cistrons are separated by nucleic acid encoding viral self-cleaving peptides, the second and third cistrons are separated by nucleic acid encoding a viral self-cleaving peptide, and the third and fourth cistrons are separated by an IRES sequence. In some embodiments, the multicistronic reporter vector comprises four cistrons wherein the first and second cistrons are separated by nucleic acid encoding viral self-cleaving peptides, the second and third cistrons are separated by nucleic acid encoding a viral self-cleaving peptide, and the third and fourth cistrons are separated by an IRES sequence. In some embodiments, the multicistronic reporter vector comprises four cistrons wherein the first and second cistrons are separated by nucleic acid encoding viral self-cleaving peptides, the second and third cistrons are separated by an IRES sequence, and the third and fourth cistrons are separated by nucleic acid encoding a viral self-cleaving peptide. In some embodiments, the multicistronic reporter vector comprises four cistrons wherein the first and second cistrons are separated by an IRES sequence, the second and third cistrons are separated by nucleic acid encoding viral self-cleaving peptides, and the third and fourth cistrons are separated by nucleic acid encoding a viral self-cleaving peptide. In some embodiments, the multicistronic reporter vector comprises four cistrons wherein the first and second cistrons are separated by an IRES sequence, the second and third cistrons are separated by an IRES sequence, and the third and fourth cistrons are separated by nucleic acid encoding a viral self-cleaving peptide. In some embodiments, the multicistronic reporter vector comprises four cistrons wherein the first and second cistrons are separated by nucleic acid encoding viral self-cleaving peptides, the second and third cistrons are separated by an IRES sequence, and the third and fourth cistrons are separated by an IRES sequence. In some embodiments, the multicistronic reporter vector comprises four cistrons wherein the first and second cistrons are separated by an IRES sequence, the second and third cistrons are separated by nucleic acid encoding viral self-cleaving peptides, and the third and fourth cistrons are separated by an IRES sequence. In some embodiments, the multicistronic reporter vectors comprises five or more cistrons wherein the cistrons are separated from each other by any combination of nucleic acid encoding viral self-cleaving peptides and IRES sequences.

In some embodiments, the multicistronic reporter vector of the inventions comprises one or more nucleic acids encoding a peptide linker between one or more of the reporter polypeptides and one or more of the self-cleaving peptides. In some embodiments, the peptide linker comprises the sequence Gly-Ser-Gly. In some embodiments, the multicistronic reporter vector comprises one peptide linker between one or more reporter polypeptides. In some embodiments, the multicistronic reporter vector comprises one peptide linker between two reporter polypeptides. In some embodiments, the multicistronic reporter vector comprises three reporter polypeptides where a first peptide linker is between a first reporter polypeptides and a second reporter polypeptide and a second peptide linker is between the second reporter polypeptide and a third reporter polypeptide. In some embodiments, the first peptide linker and/or the second peptide linker is absent from the multicistronic reporter vector. In some embodiments, the multicistronic reporter vector comprises four reporter polypeptides where a first peptide linker is between a first reporter polypeptides and a second reporter polypeptide, a second peptide linker is between the second reporter polypeptide, and a third reporter polypeptide, and a third peptide linker is between the third reporter polypeptide and a fourth reporter polypeptide. In some embodiments, the first peptide linker and/or the second peptide linker and/or the third peptide linker is absent from the multicistronic reporter vector. In some emodiments, the peptide linkers are the same peptide linker (e.g., Gly-Ser-Gly). In other embodiments, at least two of the peptide linkers in the multicistronic reporter vectors are different.

In some embodiments, the invention provides multicistronic reporter vectors comprising an open reading frame is operably linked to a promoter and wherein the open reading frame includes two or more MCS sequences linked to nucleic acid encoding a reporter polypeptide such that when nucleic acid encoding a transgene of interest is inserted into an MCS, the resulting polypeptide encoded by the multicistronic reporter vector includes the product of the transgene of interested tagged with the reporter polypeptide. Each cistron of the open reading frame encodes a different reported polypeptide such that each tagged transgene product may be profiled in a live cell. In some embodiments, the reporter polypeptide is a fluorescent reporter polypeptide. In some embodiments, the reporter polypeptide may be a green fluorescent protein (GFP) or any of its derivatives. In some embodiments the reporter polypeptide is a non GFP derived fluorescent peptide. In some embodiments the reporter polypeptide is GFP, EGFP, Emerald, Citrine, Venus, mOrange, mCherry, TagBFP, mTurquoise, Cerulean, UnaG, dsRed, eqFP611, Dronpa, RFP, TagRFPs, TdTomato, KFP, EosFP, Dendra, IrisFP, iRFPs, or smURFP. In some embodiments, the reporter polypeptide is a luciferase. In some embodiments, the reporter polypeptide is an enzyme, which when expressed allows for visualization of expression through the products of a chemical reaction. In some embodiments the reporter domain is a firefly luciferase or a Renilla luciferase. In some embodiments the reporter domain is β-glucuronidase or β-galactosidase.

In some embodiments, the invention provides a multicistronic reporter vector, wherein the vector comprises a promoter operably linked to an open reading frame, wherein the open reading frame comprises a first cistron and a second cistron, wherein each cistron comprises 5′ to 3′ nucleic acid comprising a MCS, nucleic acid encoding a reporter polypeptide, and nucleic acid encoding a linker peptide; wherein the first cistron and the second cistron are separated by nucleic acid encoding a viral cleavage peptide. In some embodiments, the invention provides a multicistronic reporter vector, wherein the vector comprises a promoter operably linked to an open reading frame, wherein the open reading frame comprises a first cistron, a second cistron and a third cistron, wherein each cistron comprises 5′ to 3′ nucleic acid comprising a MCS, nucleic acid encoding a reporter polypeptide, and nucleic acid encoding a linker peptide; wherein the first cistron and the second cistron are separated by nucleic acid encoding a first viral cleavage peptide and the second cistron and the third cistron are separated by nucleic acid encoding a second viral cleavage peptide. In some embodiments, the invention provides a multicistronic reporter vector, wherein the vector comprises a promoter operably linked to an open reading frame, wherein the open reading frame comprises a first cistron, a second cistron, a third cistron and a fourth cistron, wherein each cistron comprises 5′ to 3′ nucleic acid comprising a MCS, nucleic acid encoding a reporter polypeptide nucleic acid encoding a linker peptide; wherein the first cistron and the second cistron are separated by nucleic acid encoding a first viral cleavage peptide, the second cistron and the third cistron are separated by nucleic acid encoding a second viral cleavage peptide the third cistron and the fourth cistron are separated by nucleic acid encoding a third viral cleavage peptide. In some embodiments, the invention provides a multicistronic reporter vector, wherein the vector comprises a promoter operably linked to an open reading frame, wherein the open reading frame comprises a first cistron, a second cistron, a third cistron and a fourth cistron, wherein each cistron comprises 5′ to 3′ nucleic acid comprising a MCS, nucleic acid encoding a reporter polypeptide, nucleic acid encoding a linker peptide; wherein the first cistron and the second cistron are separated by nucleic acid encoding a first viral cleavage peptide, the second cistron and the third cistron are separated by nucleic acid encoding a second viral cleavage peptide the third cistron and the fourth cistron are separated by nucleic acid encoding an IRES.

In some embodiments, the multicistronic reporter vector further comprises one or more inducible elements located between the promoter and open reading frame. In some embodiments, the multicistronic reporter vector comprises two inducible elements. In some embodiments, the inducible element is a Tet operator 2 (TetO2) inducible element.

In some embodiments, the invention provides multicistronic reporter vectors wherein the vector comprises an open reading frame comprising two or more cistrons, wherein the open reading frame is operably linked to a promoter. In some embodiments, the promoter is a constitutive promoter. In some embodiments, the constitutive promoter is a Cytomegalovirus a (CMV), a Thymidine Kinase (TK), an eF 1-alpha, a Ubiquitin C (UbC), a Phosphoglycerate Kinase (PGK), a CAG promoter, an SV40 promoter, or a human β-actin promoter.

In some embodiments, the invention provides multicistronic reporter vectors wherein the vector comprises an open reading frame comprising two or more cistrons, wherein the open reading frame is operably linked to an inducible promoter. In some embodiments, the inducible promoter is a tetracycline responsive promoter. In some embodiments, the inducible promoter is a rapamycin-regulated promoter or a sterol inducible promoter.

In some embodiments, the invention provides multicistronic reporter vectors wherein the vector comprises an open reading frame comprising two or more cistrons, wherein the open reading frame is operably linked to a tissue specific promoter. In some embodiments, the tissue specific promoter is specific for cells of heart, blood, muscle, lung, liver, kidney, pancreas, brain, or skin.

In some embodiments, the invention provides multicistronic reporter vectors wherein the vector comprises an open reading frame comprising two or more cistrons, wherein the vector further comprises a site-specific recombinase sequence located 3′ to the open reading frame.

Examples of site-specific recombinase sequences include but are not limited to a FRT nucleic acid sequence, an attP nucleic acid sequence and loxP nucleic acid sequence. In some embodiments, the site-specific recombinase sequence is a FRT nucleic acid. In some embodiments, the site-specific recombinase sequence is a attP nucleic acid. In some embodiments, the site-specific recombinase sequence is a attB nucleic acid. In some embodiments, the site-specific recombinase sequence is a loxP nucleic acid. In some embodiments, the site-specific recombinase sequence is a FRT nucleic acid and an attP sequence. In some embodiments, the site-specific recombinase sequence is a FRT nucleic acid and an attB sequence.

In some embodiments, the invention provides multicistronic reporter vectors wherein the vector comprises an open reading frame comprising two or more cistrons, wherein the vector further comprises nucleic acid encoding a selectable marker, wherein the nucleic acid encoding the selectable marker is not operably linked to the promoter when the site-specific recombinase sequence has not recombined and is operably linked to the promoter when the site-specific recombinase sequence recombines with its target site-specific recombinase sequence. In some embodiments, the selectable marker confers resistance to hygromyocin, Zeocin™, puromycin, neomycin or an analog of hygromyocin, Zeocin™, puromycin, blasticidin or neomycin.

In some embodiments, the invention provides multicistronic reporter vectors wherein the vector comprises an open reading frame comprising two or more cistrons, wherein nucleic acid encoding one or more polypeptides is inserted in-frame into the one or more MCS. In some embodiments, the one or more polypeptides comprise polypeptides that can be used to profile a single biological pathway, cross-talk between two or more biological pathways, cellular homeostasis, organelle homeostasis or a toxicity response. In some embodiments, the one or more polypeptides include ATF4, ATF6, XBP1ADBBD and H2B; alpha-tubulin, mitochondrial targeting sequence (MTS), LC3 and H2B; or 53BP1, Nrf2, p53RE and H2B; Mek, Erk, Raf and Ras; H2B, palmitoylation signal and MTS; or H2B, MTS and alpha-actinin2. In some embodiments, the invention provides multiple multicistronic reporter vectors, wherein the multiple multicistronic reporter vectors are used to profile a specific target selected from a single biological pathway, cross-talk between two or more biological pathways, cellular homeostasis, organelle homeostasis and a toxicity response; wherein each vector encodes at least one common polypeptide (e.g., H2B) that can be used to identify cells that received one or more of the multicistronic vectors encoding polypeptides targeted to a specific target. In some respects, the common polypeptide may be considered a barcode for the specific target.

In some embodiments, the invention provides a multicistronic reporter vector as described above, wherein the vector comprises one, two or three transcription units comprising a promoter and nucleic acid encoding a transgene located 5′ to the open reading frame comprising two or more cistrons, wherein the reporter vector further comprises a core insulator sequence and a polyA sequence located 3′ to the transcription units and 5′ to the open reading frame comprising two or more cistrons. In some embodiments, the one, two, or three transcription units encode transcription factors, or other factors that may aid in the assays described herein.

In some embodiments, the invention provides multicistronic reporter vectors as described above, wherein the vector comprises nucleic acid encoding H2B fused to a reporter polypeptide and operably linked to an MLC2v promoter, an SLN promoter, or a SHOX2 promoter, thereby enabling expression of a reporter in a cardio subtype cell. In some embodiments, the invention provides multicistronic reporter vectors as described above, wherein the vector comprises nucleic acid encoding H2B fused to a reporter polypeptide and operably linked to a vGAT promoter, a TH promoter, a GFAP promoter, or a vGLUT promoter, thereby enabling expression of a reporter in a neural subtype cell.

Multireporter Cells

In some embodiments, the invention provides a multireporter cell comprising any of the acceptor cells described above in which a multicistronic reporter vector described above has integrated into the genome of the acceptor cell. In some embodiments, the multicistronic reporter vector has integrated into a specific site in an acceptor cell genome. In some embodiments, the specific site in the acceptor cell genome is an adeno-associated virus S1 (AAVS1) locus, a chemokine (CC motif) receptor 5 (CCRS) locus, a human ortholog of the mouse ROSA26 locus, a hippl 1 (H11) locus or the citrate lyase beta like gene locus (CLYBL). In some embodiments, a single copy of the multicistronic reporter vector is integrated into the acceptor cell genome.

In some embodiments, any of the multicistronic reporter vectors described above is inserted into an acceptor cell to generate a multireporter cell of the invention.

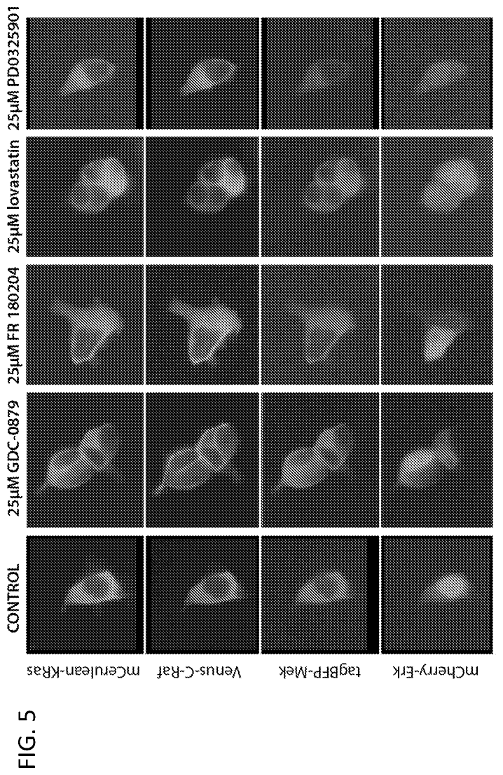

In some embodiments, the invention provides a multireporter cell, wherein the reporter cell comprises a multicistronic reporter construct, wherein the multicistronic reporter construct comprises a promoter operably linked to an open reading frame, wherein the open reading frame comprises two or more cistrons; wherein each cistron comprises a nucleic acid encoding a different transgene product fused to a different reporter polypeptide, wherein expression of the open reading frame in a cell yields separate component polypeptide products from each cistron; and wherein expression of the transgene products is essentially stoichiometric. In some embodiments, the cistrons are separated from one another by nucleic acid encoding one or more self-cleaving peptide and/or one or more internal ribosome entry site (IRES). A nonlimiting example of a reporter construct in a multireporter cell in provide in FIG. 5.

In some embodiments, the essentially stoichiometric expression is expression of two or more transgene products, wherein the expression level of the two or more transgene products is essentially the same; i.e., have 1:1 stoichiometry. In some embodiments, the expression level of the two or more reporter polypeptides are equal or vary by no more than any of about 5%, 10%, 15%, 20% or 25% of each other. In some embodiments, the essentially stoichiometric expression is the stoichiometric expression of two, three, four or more than four transgene products. Expression levels of the two or more reporter polypeptides can be measured by any means known in the art; for example, by fluorescence detection, by immunoassay, by enzyme assay, by measuring RNA levels (e.g. qPCR), etc.

In some embodiments, the cistrons of the multicistronic reporter vector inserted in the multireporter cell are separated from one another by nucleic acid encoding one or more self-cleaving peptide and/or one or more internal ribosome entry site (IRES). In some embodiments, the one or more self-cleaving peptides is a viral self-cleaving peptide. In some embodiments, the one or more viral self-cleaving peptides is one or more 2A peptides. In some embodiments, the one or more 2A peptides is a T2A peptide, a P2A peptide, an E2A peptide or a F2A peptide. In some embodiments, one or more of the cistrons of the open reading frame is separated from the other cistrons in the open reading frame by an IRES sequence. In some embodiments, the IRES is an encephalomyocarditis virus (EMCV) IRES, a Hepatitis C virus (HCV) IRES or an Enterovirus 71 (EV71) IRES.

In some embodiments, the multireporter cell comprises a multicistronic reporter vector wherein the multicistronic reporter vector comprises two cistrons wherein the two cistrons are separated by nucleic acid encoding a viral self-cleaving peptide or an IRES. In some embodiments, the multicistronic reporter vector comprises three cistrons wherein the three cistrons are separated by nucleic acid encoding viral self-cleaving peptides. In some embodiments, the multicistronic reporter vector comprises three cistrons wherein the three cistrons are separated by IRES sequences. In some embodiments, the multicistronic reporter vector comprises three cistrons wherein the first and second cistrons are separated by nucleic acid encoding a viral self-cleaving peptide and the second and third cistron is separated by an IRES sequence. In some embodiments, the multicistronic reporter vector comprises three cistrons wherein the first and second cistrons are separated by an IRES sequence and the second and third cistron is separated by nucleic acid encoding a self-cleaving peptide. In some embodiments, the multicistronic reporter vector comprises four cistrons wherein the cistrons are separated from one another by nucleic acid encoding viral self-cleaving peptides. In some embodiments, the multicistronic reporter vector comprises four cistrons wherein the cistrons are separated from one another by IRES sequences. In some embodiments, the multicistronic reporter vector comprises four cistrons wherein the first and second cistrons are separated by nucleic acid encoding viral self-cleaving peptides, the second and third cistrons are separated by nucleic acid encoding a viral self-cleaving peptide, and the third and fourth cistrons are separated by an IRES sequence. In some embodiments, the multicistronic reporter vector comprises four cistrons wherein the first and second cistrons are separated by nucleic acid encoding viral self-cleaving peptides, the second and third cistrons are separated by nucleic acid encoding a viral self-cleaving peptide, and the third and fourth cistrons are separated by an IRES sequence. In some embodiments, the multicistronic reporter vector comprises four cistrons wherein the first and second cistrons are separated by nucleic acid encoding viral self-cleaving peptides, the second and third cistrons are separated by an IRES sequence, and the third and fourth cistrons are separated by nucleic acid encoding a viral self-cleaving peptide. In some embodiments, the multicistronic reporter vector comprises four cistrons wherein the first and second cistrons are separated by an IRES sequence, the second and third cistrons are separated by nucleic acid encoding viral self-cleaving peptides, and the third and fourth cistrons are separated by nucleic acid encoding a viral self-cleaving peptide. In some embodiments, the multicistronic reporter vector comprises four cistrons wherein the first and second cistrons are separated by an IRES sequence, the second and third cistrons are separated by an IRES sequence, and the third and fourth cistrons are separated by nucleic acid encoding a viral self-cleaving peptide. In some embodiments, the multicistronic reporter vector comprises four cistrons wherein the first and second cistrons are separated by nucleic acid encoding viral self-cleaving peptides, the second and third cistrons are separated by an IRES sequence, and the third and fourth cistrons are separated by an IRES sequence. In some embodiments, the multicistronic reporter vector comprises four cistrons wherein the first and second cistrons are separated by an IRES sequence, the second and third cistrons are separated by nucleic acid encoding viral self-cleaving peptides, and the third and fourth cistrons are separated by an IRES sequence. In some embodiments, the multicistronic reporter vectors comprises five or more cistrons wherein the cistrons are separated from each other by any combination of nucleic acid encoding viral self-cleaving peptides and IRES sequences.

In some embodiments, the multireporter cell comprises a multicistronic reporter vector which comprises one or more nucleic acids encoding a peptide linker between one or more of the reporter polypeptides and one or more of the self-cleaving peptides. In some embodiments, the peptide linker comprises the sequence Gly-Ser-Gly.

In some embodiments, the invention provides multireporter cells comprising a multicistronic reporter vector which comprises an open reading frame is operably linked to a promoter and wherein the open reading frame includes two or more MCS sequences linked to nucleic acid encoding a reporter polypeptide such that when nucleic acid encoding a transgene of interest is inserted into an MCS, the resulting polypeptide encoded by the multicistronic reporter vector includes the product of the transgene of interested tagged with the reporter polypeptide.

Each cistron of the open reading frame encodes a different reported polypeptide such that each tagged transgene product may be profiled in a live cell. In some embodiments, the reporter polypeptide is a fluorescent reporter polypeptide. In some embodiments, the reporter polypeptide may be a green fluorescent protein (GFP) or any of its derivatives. In some embodiments the reporter polypeptide is a non GFP derived fluorescent peptide. In some embodiments the reporter polypeptide is GFP, EGFP, Emerald, Citrine, Venus, mOrange, mCherry, TagBFP, mTurquoise, Cerulean, UnaG, dsRed, eqFP611, Dronpa, RFP, TagRFPs, TdTomato, KFP, EosFP, Dendra, IrisFP, iRFPs, or smURFP. In some embodiments, the reporter polypeptide is a luciferase. In some embodiments, the reporter polypeptide is an enzyme, which when expressed allows for visualization of expression through the products of a chemical reaction. In some embodiments the reporter domain is a firefly luciferase or a Renilla luciferase. In some embodiments the reporter domain is β-glucuronidase or β-galactosidase.

In some embodiments, the invention provides a multireporter cell comprising a multicistronic reporter vector, wherein the vector comprises a promoter operably linked to an open reading frame, wherein the open reading frame comprises a first cistron and a second cistron, wherein each cistron comprises 5′ to 3′ nucleic acid comprising a MCS, nucleic acid encoding a reporter polypeptide, and nucleic acid encoding a linker peptide; wherein the first cistron and the second cistron are separated by nucleic acid encoding a viral cleavage peptide. In some embodiments, the invention provides a multicistronic reporter vector, wherein the vector comprises a promoter operably linked to an open reading frame, wherein the open reading frame comprises a first cistron, a second cistron and a third cistron, wherein each cistron comprises 5′ to 3′ nucleic acid comprising a MCS, nucleic acid encoding a reporter polypeptide, and nucleic acid encoding a linker peptide; wherein the first cistron and the second cistron are separated by nucleic acid encoding a first viral cleavage peptide and the second cistron and the third cistron are separated by nucleic acid encoding a second viral cleavage peptide. In some embodiments, the invention provides a multicistronic reporter vector, wherein the vector comprises a promoter operably linked to an open reading frame, wherein the open reading frame comprises a first cistron, a second cistron, a third cistron and a fourth cistron, wherein each cistron comprises 5′ to 3′ nucleic acid comprising a MCS, nucleic acid encoding a reporter polypeptide nucleic acid encoding a linker peptide; wherein the first cistron and the second cistron are separated by nucleic acid encoding a first viral cleavage peptide, the second cistron and the third cistron are separated by nucleic acid encoding a second viral cleavage peptide the third cistron and the fourth cistron are separated by nucleic acid encoding a third viral cleavage peptide. In some embodiments, the invention provides a multicistronic reporter vector, wherein the vector comprises a promoter operably linked to an open reading frame, wherein the open reading frame comprises a first cistron, a second cistron, a third cistron and a fourth cistron, wherein each cistron comprises 5′ to 3′ nucleic acid comprising a MCS, nucleic acid encoding a reporter polypeptide, nucleic acid encoding a linker peptide; wherein the first cistron and the second cistron are separated by nucleic acid encoding a first viral cleavage peptide, the second cistron and the third cistron are separated by nucleic acid encoding a second viral cleavage peptide the third cistron and the fourth cistron are separated by nucleic acid encoding an IRES.

In some embodiments, the multicistronic reporter vector of the multireporter cell further comprises one or more inducible elements located between the promoter and open reading frame. In some embodiments, the multicistronic reporter vector comprises two inducible elements. In some embodiments, the inducible element is a Tet operator 2 (TetO2) inducible element.

In some embodiments, the invention provides multireporter cells comprising a multicistronic reporter vector wherein the vector comprises an open reading frame comprising two or more cistrons, wherein the open reading frame is operably linked to a promoter. In some embodiments, the promoter is a constitutive promoter. In some embodiments, the constitutive promoter is a Cytomegalovirus a (CMV), a Thymidine Kinase (TK), an eF1-alpha, a Ubiquitin C (UbC), a Phosphoglycerate Kinase (PGK), a CAG promoter, an SV40 promoter, or a human β-actin promoter.

In some embodiments, the invention provides multireporter cells comprising a multicistronic reporter vector wherein the vector comprises an open reading frame comprising two or more cistrons, wherein the open reading frame is operably linked to an inducible promoter. In some embodiments, the inducible promoter is a tetracycline responsive promoter. In some embodiments, the inducible promoter is a rapamycin-regulated promoter or a sterol inducible promoter.

In some embodiments, the invention provides a multireporter cell which comprises a multicistronic reporter vector wherein the vector comprises an open reading frame comprising two or more cistrons, wherein the open reading frame is operably linked to a tissue specific promoter. In some embodiments, the tissue specific promoter is specific for cells of heart, blood, muscle, lung, liver, kidney, pancreas, brain, or skin.

In some embodiments, the invention provides multireporter cells comprising a multicistronic reporter vector wherein the vector comprises an open reading frame comprising two or more cistrons, wherein the vector further comprises a site-specific recombinase sequence located 3′ to the open reading frame which was used to target the multicistronic reporter vector to a specific site in the cell. Examples of site-specific recombinase sequences include but are not limited to a FRT nucleic acid sequence, an attP nucleic acid sequence and loxP nucleic acid sequence. In some embodiments, the site-specific recombinase sequence is a FRT nucleic acid. In some embodiments, the site-specific recombinase sequence is an attP nucleic acid. In some embodiments, the site-specific recombinase sequence is an attB nucleic acid. In some embodiments, the site-specific recombinase sequence is a loxP nucleic acid. In some embodiments, the site-specific recombinase sequence is a FRT nucleic acid and an attP sequence. In some embodiments, the site-specific recombinase sequence is a FRT nucleic acid and an attB sequence.

In some embodiments, the invention provides multireporter cells which comprise a multicistronic reporter vector wherein the vector comprises an open reading frame comprising two or more cistrons, wherein the vector further comprises nucleic acid encoding a selectable marker, wherein the nucleic acid encoding the selectable marker is not operably linked to the promoter when the site-specific recombinase sequence has not recombined and is operably linked to the promoter when the site-specific recombinase sequence recombines with its target site-specific recombinase sequence. In some embodiments, the selectable marker confers resistance to hygromyocin, Zeocin™, puromycin, neomycin or an analog of hygromyocin, Zeocin™, puromycin, blasticidin or neomycin.

In some embodiments, the invention provides multireporter cells comprising a multicistronic reporter vector wherein the vector comprises an open reading frame comprising two or more cistrons, wherein each cistron encodes a transgene product fused to a reporter polypeptide. In some embodiments, the transgenes encode polypeptides that can be used to profile a single biological pathway, cross-talk between two or more biological pathways, cellular homeostasis, organelle homeostasis or a toxicity response. In some embodiments, the polypeptides include ATF4, ATF6, XBP1ADBBD and H2B; alpha-tubulin, mitochondrial targeting sequence (MTS), LC3 and H2B; or 53BP1, Nrf2, p53RE and H2B; Mek, Erk, Raf and Ras; H2B, palmitoylation signal and MTS; or H2B, MTS and alpha-actinin2.

In some embodiments, the invention provides a multireporter cells which comprise a multicistronic reporter vector as described above, wherein the vector comprises one, two or three additional transcription units comprising a promoter and nucleic acid encoding a transgene located 5′ to the open reading frame comprising two or more cistrons, wherein the reporter vector further comprises a core insulator sequence and a polyA sequence located 3′ to the transcription units and 5′ to the open reading frame comprising two or more cistrons. In some embodiments, the one, two, or three transcription units encode transcription factors, or other factors that may aid in the assays described herein. In some embodiments, at least of the additional transcription units comprises nucleic acid encoding a reporter molecule operably linked to a promoter.

In some embodiments, the multireporter cell may be a stem cell which can be differentiated into different lineages. For example the multireporter cell can be a totipotent, pluripotent, multipotent or progenitor stem cell. In some embodiments the multireporter cell is a totipotent stem cell which has the ability to at least differentiate into all embryonic and extraembryonic lineages. In some embodiments the multireporter cell is a pluripotent stem cell. In some embodiments the reporter pluripotent stem cell is an embryonic pluripotent stem cell isolated from an animal. In a particular embodiment the reporter pluripotent stem cell is a mammalian embryonic stem cell. In some embodiments the reporter pluripotent stem cell is a human embryonic stem cell. In some embodiments the reporter pluripotent stem cell is an induced pluripotent stem cell. The iPS cell used to develop the reporter iPS cell may have been generated by reprograming via transfection, piggy-Bac, episomal, or protein reprogramming methods. The iPS used to develop the reporter iPS cell may have been generated by reprogramming a somatic, terminally differentiated or partially differentiated cell of an ectodermal, endodermal, mesodermal, placental, chorionic, or trophectodermal lineage. For example the reporter iPS cell may have been derived from a fibroblast, a peripheral blood cell, a cord blood endothelial cell, a cord blood stem cell, an adipose-derived stem cell, a hepatocyte, a keratinocyte, a neural stem cell, a pancreatic beta-cell or an amniotic cell. The reporter iPS cell may have been derived from an established iPS cell line, or from a patient specific iPS cell. In some embodiments the reporter iPS cell was derived from an iPS cell generated by reprogramming an immune privileged cell.

In some embodiments the multireporter cell is a multipotent or a progenitor cell. The multireporter cell may be a be a hematopoietic cell, as endothelial progenitor cell, a mesenchymal progenitor cell, a neural progenitor cell, an osteochondral progenitor cell, a lymphoid progenitor cell or a pancreatic progenitor cell. In some embodiments the multireporter cell is a cord endothelial cell, a cord blood stem cell, an adipose-derived stem cell, a hepatocyte, a keratinocyte, a neural stem cell, a pancreatic beta-cell or an amniotic cell.

The stem cell may be differentiated into any progenitor or terminal cell lineage. Methods of general or lineage specific differentiation are known in the art. The stem cell may be differentiated using any methods known in the art. For example the stem cell may be differentiated using one or more factors or molecules that drive differentiation, one or more cellular matrixes, embryoid body formation, or a combination of the above. In some embodiments, the differentiated cell is a multireporter cell.

The cells may be differentiated into cardiomyocytes, endothelial cells, neuronal cells, GABAergic neurons, astrocytes, dopaminergic neurons, glutamatergic neurons, hepatocytes, hepatoblasts, skeletal myoblasts, macrophages, cortical neurons, atrial cardiomyocytes, ventricular cardiomyocytes, Purkinje fibers, basal cells, squamous cells, renal cells, pancreatic beta cells, epithelial cells, mesenchymal cells, adrenocortical cells, osteoblasts, osteocytes, chondroblasts, chondrocytes, gastrointestinal cells, colorectal cells, ductal cells, lobular cells, lymphocytes, retinal cells, photoreceptor cells or cochlear cells.

In some embodiments the reporter iPS cells comprises a multicistronic reporter as described in any of the embodiments above, wherein the multicistronic reporter is driven by the promoter which is active only in pluripotent cells. For example the promoter operably linked to the open reading frame comprising two or more cistrons, can be Oct-4, Sox2, Nanog, KLF4, TRA-1-60, TRA-2-54, TRA-1-81, SSEA1, SSEA4 or the promoter of any pluripotency associated gene.

Toxicity can be tested by monitoring expression and, or movement within a cell or between cells of various peptides associated with toxicity. For example expression and movement of proteins involved in unfolded protein response, autophagy, DNA damage, oxidative stress and p53-dependent stress response.

In some embodiments the multireporter cell is an immortal cell. For example the reporter may be a HEK293T cell, an A549 cell, an U2OS cell, an RPE cell, an NPC1 cell, a MCF7 cell, a HepG2 cell, a HaCat cell, a TK6 cell, an A375 cell or a HeLa cell. In some embodiments, the multireporter cell is a primary cell.

A multireporter cell may be used to test the toxicity, to test and monitor the effects of various molecules in cells, to test effects of different therapies in cells or to monitor movement of proteins in cells in response to a stimulus. Example of molecules and therapies include chemicals, chemical compositions, small biologics, nanoparticles, peptides, antibodies, vaccines and combinations thereof.

In some embodiments, the invention provides methods for generating a multireporter cell, the method comprising introducing the multicistronic reporter vector described herein into any acceptor cell as described herein. In some embodiments, the multispecific reporter vector is inserted into the acceptor site of the acceptor cell recombinase system. In some embodiments, the multicistronic reporter vector comprises a recombinase associated nucleic acid which can insert into a recombinase associated nucleic acid of the acceptor cell by way of a recombinase protein in the acceptor cell. In some embodiments, the nucleic acid encoding the recombinase protein is stably introduced in the acceptor cell. In some embodiments, the nucleic acid encoding the recombinase is transiently introduced into the acceptor cell. In some embodiments, the nucleic acid encoding the recombinase is transiently introduced into the acceptor cell before, at the same time or after introduction of the multicistronic reporter vector. In some embodiments, the recombinase protein is introduced into the acceptor cell. In some embodiments, the recombinase associated nucleic acid sequence is FRT nucleic acid sequence and the acceptor cell comprises a flp recombinase. In some embodiments, the recombinase associated nucleic acid is attP and the acceptor cell comprises a Bxbl recombinase, a PhiC31 recombinase, or R4 recombinase. In some embodiments, the recombinase associated nucleic acid sequence is loxP nucleic acid sequence and the acceptor cell comprises a CRE recombinase.

Libraries

In some aspects, the invention provides one or more libraries of multicistronic reporter vectors, wherein the library comprises multicistronic reporter molecules comprising different transgenes encoding polypeptides fused to reporter polypeptides, wherein two or more of the different transgenes on each vector are expressed essentially stoichiometrically when introduced to cells. In some embodiments, the library comprises reporter vectors that encode one or more transgenes encode polypeptides that can be used to profile a single biological pathway, cross-talk between two or more biological pathways, cellular homeostasis, organelle homeostasis, a toxicity response and/or phenotypic features. In some embodiments, the library is used to visualize biological pathways or targets readouts with phenotypic readouts. In some embodiments, the library comprises two or more different multicistronic reporter vectors. In some embodiments, the library comprises between any of about two and about 10, about 10 and about 20, about 20 and about 30, about 30 and about 40, about 40 and about 50, about 50 and about 100, about 100 and about 500, about 500 and about 100, about 1000 and about 10,000 different multicistronic reporter vectors. In some embodiments, the library comprises greater than about 10,000 different multicistronic reporter vectors

In some aspects, the invention provides one or more libraries of multicistronic reporter cells, wherein the library comprises a plurality of multicistronic reporter cells comprising different transgenes encoding polypeptides fused to reporter polypeptides, wherein two or more of the different transgenes on each vector are expressed essentially stoichiometrically when introduced to cells. In some embodiments, the library comprises a plurality of reporter cells that encode one or more transgenes encoding polypeptides that can be used to profile a single biological pathway, cross-talk between two or more biological pathways, cellular homeostasis, organelle homeostasis or a toxicity response. In some embodiments, the library comprises between any of about two and about 10, about 10 and about 20, about 20 and about 30, about 30 and about 40, about 40 and about 50, about 50 and about 100, about 100 and about 500, about 500 and about 100, about 1000 and about 10,000 different multicistronic reporter cells. In some embodiments, the library comprises greater than about 10,000 different multicistronic reporter cells. In some embodiments, the plurality of multicistronic reporter cells are isogenic; i.e., derived from a common acceptor cell to which different multicistronic vectors were introduced.

In some embodiments, the essentially stoichiometric expression is expression of two or more transgenes wherein the expression level of the two or more transgenes is essentially the same; i.e., have 1:1 stoichiometry. In some embodiments, the expression level of the two or more reporter polypeptides are equal or vary by no more than any of about 5%, 10%, 15%, 20% or 25% of each other. In some embodiments, the essentially stoichiometric expression is the stoichiometric expression of two, three, four, or more than four transgenes of the multicistronic reporter vectors. Expression levels of the two or more transgenes can be measured by any means known in the art; for example, by fluorescence detection, by immunoassay, by enzyme assay, by measuring RNA levels (e.g. qPCR), etc.

In some embodiments, the library comprises multicistronic reporter vectors to profile a biological pathway associated with a disease. In some embodiments, the disease is cancer, a cardiovascular disease, a neurodegenerative disease or an autoimmune disease. In some embodiments, the biological pathway is a pathway associated with toxic response mechanism within the cell. In some embodiments, the library comprises multicistronic reporter vectors to profile biological pathway associated with cell proliferation, cell differentiation, cell death, apoptosis, autophagy, DNA damage and repair, or oxidative stress, chromatin/epigenetics (e.g. chromatin acetylation), MAPK signaling (e.g MAPK/Erk), PI3K/Akt signaling (e.g. mTor signaling), translational control (e.g. eIF2 regulation), cell cycle and checkpoint control (Gl/S checkpoint), cellular metabolism (e.g. insulin receptor signaling), development and differentiation signaling (e.g. Wnt signaling), immunology and inflammation signaling (e.g. JAK/STAT signaling), tyrosine kinase signaling (e.g. ErbB/HER signaling), vesicle trafficking, cytoskeletal regulation or protein degradation (e.g. ubiquitin pathway) and any synthetically lethal combinations of these pathways. In some embodiments, each multicistronic vector comprising transgenes used to profile a specific single biological pathway, specific cross-talk between two or more biological pathways, a specific cellular homeostasis, a specific organelle homeostasis or a specific toxicity response of the library comprises a common transgene encoding a polypeptide fused to a reporter polypeptide. Such common reporter transgenes can be used to identify cells that receive multicistronic reporter vectors directed toward a common profile target.

In some embodiments, the invention provides libraries of acceptor cells for receiving multicistronic reporter vectors. In some embodiments, the library comprises two or more different acceptor cells as described herein. In some embodiments, the library comprises between any of about two and about 10, about 10 and about 20, about 20 and about 30, about 30 and about 40, about 40 and about 50, about 50 and about 100, about 100 and about 500 different acceptor cells. In some embodiments, the library comprises more than about 500 different acceptor cells. In some embodiments, a common multicistronic reporter vector can be introduced into two or more acceptor cells to compare profiles in different cellular backgrounds.

In some embodiments, the invention provides libraries of multireporter cells, wherein each cell in the library comprises a multicistronic reporter vector comprising different transgenes encoding polypeptides fused to reporter polypeptides, wherein the different transgenes on each vector are expressed essentially stoichiometrically when introduced to cells. In some embodiments, the library comprises between any of about two and about 10, about 10 and about 20, about 20 and about 30, about 30 and about 40, about 40 and about 50, about 50 and about 100, about 100 and about 500 different multireporter cells. In some embodiments, different multicistronic reporter vectors that target a common pathway share a common reporter polypeptide as a means for identifying cells that received related multicistronic reporter vectors.

In some embodiments, the library of acceptor cells and/or the library of multireporter cells comprises different immortalized cells. In some embodiments, the library includes one or more of a HEK293T cell, an A549 cell, an U2OS cell, an RPE cell, an NPC1 cell, a MCF7 cell, a HepG2 cell, a HaCat cell, a TK6 cell, an A375 cell or a HeLa cell.

In some embodiments, the library of acceptor cells and/or the library of multireporter cells comprises different pluripotent, multipotent and/or progenitor cells. In some embodiments, the different pluripotent or multipotent cells include one or more of an induced pluripotent stem cell, a multipotent cell, a hematopoietic cell, an endothelial progenitor acceptor cell, a mesenchymal progenitor cell, a neural progenitor cell, an osteochondral progenitor cell, a lymphoid progenitor cell or a pancreatic progenitor cell. In some embodiments, the library of pluripotent or multipotent cells multireporter cells is differentiated after introduction of the multicistronic reporter vector. In some embodiments, the library includes one or more of a WTC-11 iPSC or an NCRM5 iPSC.

In some embodiments, the library of acceptor cells and/or the library of multireporter cells comprises different primary cells. In some embodiments, the primary cells comprise one or more of a cardiomyocyte, a muscle cell, a lung cell, a liver cell, a kidney cell, a pancreatic cell, a neuron, or a tumor cell.

In some embodiments, each cell in the library of multireporter cells comprises the same multicistronic reporter vector. In other embodiments, cells in the library of multireporter cells comprise different multicistronic reporter vectors. In some embodiments, the different multicistronic reporter vectors were introduced to isogenic acceptor cells.

In some embodiments, the invention provides libraries of multireporter cells wherein the reporter cells comprise a multicistronic reporter vectors encoding one or more polypeptides fused to a reporter polypeptide that can be used to profile a single biological pathway, cross-talk between two or more biological pathways, or cellular homeostasis. In some embodiments, the biological pathway is a pathway associated with a disease. In some embodiments, the disease is cancer, a cardiovascular disease, a neurodegenerative disease or an autoimmune disease. In some embodiments, the biological pathway is a pathway associated with toxic response mechanism within the cell. In some embodiments, the biological pathway is a pathway associated with cell proliferation, cell differentiation, cell death, apoptosis, autophagy, DNA damage and repair, or oxidative stress, chromatin/epigenetics (e.g. chromatin acetylation), MAPK signaling (e.g MAPK/Erk), PI3K/Akt signaling (e.g. mTor signaling), translational control (e.g. eIF2 regulation), cell cycle and checkpoint control (Gl/S checkpoint), cellular metabolism (e.g. insulin receptor signaling), development and differentiation signaling (e.g. Wnt), immunology and inflammation signaling (e.g. JAK/STAT signaling), tyrosine kinase signaling (e.g. ErbB/HER signaling), vesicle trafficking, cytoskeletal regulation or protein degradation (e.g. ubiquitin pathway) and synthetically lethal combinations of these pathways. In some embodiments, the library of multireporter cells comprise different multicistronic vector comprising transgenes used to profile a specific single biological pathway, specific cross-talk between two or more biological pathways, a specific cellular homeostasis, a specific organelle homeostasis or a specific toxicity response wherein each different multicistronic reporter vectors comprises a common transgene fused to nucleic acid encoding a reporter polypeptide. In some embodiments, the common transgene product fused to a reporter polypeptide is used as a means for identifying cells that received related multicistronic reporter vectors.

Assays

The invention provides live cell assays using the cells and vectors described herein. In some embodiments, the assay is performed on a single live cell. In some embodiments, the invention provides a method of profiling two or more polypeptides in a live cell, the method comprising determining the expression of the two or more of the transgenes and/or location of the two or more transgene products of a multireporter cell as described herein. In some embodiment, the method is used to profile a single biological pathway, cross-talk between two or more biological pathways, cellular homeostasis, organelle homeostasis, a toxicity response and/or phenotypic features. In some embodiments, the assays are used to visualize biological pathways or targets readouts with phenotypic readouts. In some embodiments, the expression of the two or more of the transgenes and/or location of the two or more transgene products is determined at one or more time points. In some embodiments, the expression of the two or more of the transgenes and/or location of the two or more transgene products is determined at one or more of 1 minute, 5 minutes, 10 minutes, 15 minutes, 30 minutes, 45 minutes, 1 hour, 2 hours, 3 hours, 4 hours, 5 hours, 6 hours, 12 hours, 24 hours, 2 days, 4 days, 7 days, 14 days, 21 days, 30 days, 1 month, 3 month, 6 month, 9 month, 1 year, or any time there between or more than 1 year after initiation of the analysis.

In some embodiments, the location of a transgene product is one or more of a cell membrane, a cell nucleus, a cell nuclear membrane, a mitochondria, a mitochondrial membrane, an autophagosome, a lysosome, an endosome, a golgi, or a cytosol.

In some embodiments, the invention provides methods to measure the effects of an agent on the profile of two or more polypeptides in a live cell, the method comprising subjecting a multireporter cell as described herein to the agent and determining the expression of the two or more of the transgenes and/or location of the two or more transgene products in the cell in response to the agent. In some embodiments, the agent is a drug or drug candidate. In some embodiments, the agent is a cancer drug or cancer drug agent. In some embodiments, the method is a toxicology screen.

In some embodiments, the location of a transgene product is one or more of a cell membrane, a cell nucleus, a cell nuclear membrane, a mitochondria, a mitochondrial membrane, an autophagosome, a lysosome, an endosome, a golgi, or a cytosol.

In some embodiments of the above assays and methods, the profile is obtained from a single live cell. In some embodiments, the profile is determined for multiple live cells. In some embodiments, the cells are culture on a tissue culture plate, including but not limited to multiwell tissue culture plates such as a 96-well or a 384-well tissue culture plate. In some embodiments, the cells are in a suspension culture.

In some embodiments of the above assays and methods, determining the expression of the two or more of the transgenes and/or location of the two or more transgene products is performed in a library of multireporter cells.

In some embodiments, the expression and/or location of the two or more polypeptides is measured by microscopy, high throughput microscopy, fluorescence-activated cell sorting (FACS), luminescence, using a plate reader, mass spectrometry, or deep sequencing.

In some embodiments, the profiles are configured into a panel of three multicolor reporters, directed toward a common cellular response. These reporters enable 10 distinct fluorescent readouts that discriminate aspects of the cellular response. All three reporters have H2B as a DNA/nucleus marker that we will label with spectrally different fluorescent markers, in order to enable their spectral discrimination when used in pooled assays. Pooling isogenic reporter cells in the same well enables highly multiplexed assays of at least 10 readouts since the H2B/nuclear marker permits automated image analysis clustering by reporter.

In some embodiments, the profiles are configured into a panel of three multicolor ‘Tox’ reporters, directed toward unfolded protein response panel, organelle panel and DNA damage, oxidative and p53 stress panel. These reporters enable 10 distinct fluorescent readouts that discriminate early stress responses: oxidative stress, UPR and p53-dependent cellular stress response from late stress responses: DNA double-strand breaks, autophagy, cell cycle and overall nucleus and mitochondria homeostasis. All three Tox reporters have H2B as a DNA/nucleus marker that we will label with spectrally different fluorescent markers, in order to enable their spectral discrimination when used in pooled assays. Pooling isogenic reporter cells in the same well enables highly multiplexed assays of at least 10 readouts since the H2B/nuclear marker permits automated image analysis clustering by reporter.

Kits and Articles of Manufacture

In some embodiments, the invention provides a kit comprising one or more multicistronic reporter vectors as described herein. In some embodiments, the invention provides a kit comprising one or more acceptor cells as described herein. In some embodiments, the invention provides a kit comprising one or more of the multireporter cells described herein. In some embodiments, the invention provides a kit comprising one or more multicistronic reporter vectors described herein and one or more acceptor cells as described herein. In some embodiments, the kit further comprises instructions for using the multicistronic reporter vectors, acceptor cells and/or multireporter cells described herein. In some embodiments, the kit comprises a mixture of isogenic, but differentially labeled, multireporter cells. Such cells enable directed plating of a pooled assay.

In some embodiments, the invention provides a library of acceptor cells and/or reporter cells arrayed in a multiwell plate (e.g., a 96 well plate or 384 well plate). In some embodiments, the cells in the multiwell plate are cryopreserved.

The multicistronic reporter vectors, acceptor cells and/or multireporter cells described herein may be contained within an article of manufacture. The article of manufacture may comprise a container containing the multicistronic reporter vectors, acceptor cells and/or multireporter cells described herein. In some embodiments, the article of manufacture comprises:(a) a container comprising multicistronic reporter vectors, acceptor cells and/or multireporter cells described herein within the container; and (b) a package insert with instructions for using the multicistronic reporter vectors, acceptor cells and/or multireporter cells described herein.

In some embodiments, the article of manufacture comprises a container and a label or package insert on or associated with the container. Suitable containers include, for example, bottles, vials, syringes, etc. The containers may be formed from a variety of materials such as glass or plastic. The article of manufacture may further include other materials desirable from a commercial and user standpoint, including other buffers, diluents, reagents, tissue culture media, filters, needles, and syringes

In some embodiments, the invention provides a kit comprising a library of acceptor cells or reporter cells arrayed in a multiwell plate. In some embodiments the cells are plated cryopreserved.

### Exemplary Embodiments

1. A multicistronic reporter vector comprising: a promoter operably linked to an open reading frame, wherein the open reading frame comprises two or more cistrons, and wherein expression of the open reading frame in a cell yields separate component polypeptide products from each cistron; wherein each cistron comprises a multiple cloning site (MCS) and nucleic acid encoding a reporter vector, and wherein each cistron encodes a different reporter polypeptide; and wherein expression of two or more nucleic acids encoding polypeptides inserted into the two or more multiple cloning sites is essentially stoichiometric.

2. The multicistronic reporter vector of embodiment 1, wherein the cistrons are separated from one another by a nucleic acid encoding one or more self-cleaving peptide and/or one or more internal ribosome entry site (IRES).

3. The multicistronic reporter vector of embodiment 2, wherein the one or more self-cleaving peptides is a viral self-cleaving peptide.

4. The multicistronic reporter vector of embodiment 3, wherein the one or more viral self-cleaving peptides is one or more 2A peptides.

5. The multicistronic reporter vector of embodiment 4, wherein one or more 2A peptides is a T2A peptide, a P2A peptide, an E2A peptide or a F2A peptide.

6. The multicistronic reporter vector of any one of embodiments 2-5, wherein the reporter polypeptide further comprises one or more nucleic acids encoding a peptide linker between one or more of the reporter polypeptides and one or more of the self-cleaving peptides.

7. The multicistronic reporter vector of embodiment 6, wherein the peptide linker comprises the sequence Gly-Ser-Gly.

8. The multicistronic reporter vector of any one of embodiments 1-7, wherein the reporter polypeptide is a fluorescent reporter polypeptide.

9. The multicistronic reporter vector of any one of embodiments 1-8, wherein the reporter polypeptide for each cistron is selected from GFP, EGFP, Emerald, Citrine, Venus, mOrange, mCherry, TagBFP, mTurquoise, Cerulean, UnaG, dsRed, eqFP611, Dronpa, RFP, TagRFPs, TdTomato, KFP, EosFP, Dendra, IrisFP, iRFPs, and smURFP.

10. The multicistronic reporter vector of any one of embodiments 1-9, wherein the vector comprises a promoter operably linked to an open reading frame, wherein the open reading frame comprises a first cistron and a second cistron, wherein each cistron comprises 5′ to 3′ nucleic acid comprising a MCS, nucleic acid encoding a reporter polypeptide, and nucleic acid encoding a linker peptide; wherein the first cistron and the second cistron are separated by nucleic acid encoding a viral cleavage peptide.

11. The multicistronic reporter vector of any one of embodiments 1-9, wherein the vector comprises a promoter operably linked to an open reading frame, wherein the open reading frame comprises a first cistron, a second cistron and a third cistron, wherein each cistron comprises 5′ to 3′ nucleic acid comprising a MCS, nucleic acid encoding a reporter polypeptide, and nucleic acid encoding a linker peptide; wherein the first cistron and the second cistron are separated by nucleic acid encoding a first viral cleavage peptide and the second cistron and the third cistron are separated by nucleic acid encoding a second viral cleavage peptide.

12. The multicistronic reporter vector of any one of embodiments 1-9, wherein the vector comprises a promoter operably linked to an open reading frame, wherein the open reading frame comprises a first cistron, a second cistron, a third cistron and a fourth cistron, wherein each cistron comprises 5′ to 3′ nucleic acid comprising a MCS, nucleic acid encoding a reporter polypeptide nucleic acid encoding a linker peptide; wherein the first cistron and the second cistron are separated by nucleic acid encoding a first viral cleavage peptide, the second cistron and the third cistron are separated by nucleic acid encoding a second viral cleavage peptide the third cistron and the fourth cistron are separated by nucleic acid encoding a third viral cleavage peptide.

13. The multicistronic reporter vector of any one of embodiments 1-9, wherein the vector comprises a promoter operably linked to an open reading frame, wherein the open reading frame comprises a first cistron, a second cistron, a third cistron and a fourth cistron, wherein each cistron comprises 5′ to 3′ nucleic acid comprising a MCS, nucleic acid encoding a reporter polypeptide, nucleic acid encoding a linker peptide; wherein the first cistron and the second cistron are separated by nucleic acid encoding a first viral cleavage peptide, the second cistron and the third cistron are separated by nucleic acid encoding a second viral cleavage peptide the third cistron and the fourth cistron are separated by nucleic acid encoding an IRES.

14. The multicistronic reporter vector of any one of embodiments 1-13, wherein the vector further comprises one or more inducible elements located between the promoter and open reading frame.

15. The multicistronic reporter vector of embodiment 14, wherein the vector comprises two inducible elements.

16. The multicistronic reporter vector of embodiment 14 or 15, wherein the inducible element is a Tet operator 2 (TetO2) inducible element.

17. The multicistronic reporter vector of any one of embodiments 1-16 wherein the promoter is a constitutive promoter.

18. The multicistronic reporter vector of embodiment 17 wherein the constitutive promoter is a Cytomegalovirus a (CMV), a Thymidine Kinase (TK), an eF1-alpha, a Ubiquitin C (UbC), a Phosphoglycerate Kinase (PGK), a CAG promoter, an SV40 promoter, or a human β-actin promoter.

19. The multicistronic vector of any one of embodiments 1-16, wherein the promoter is an inducible promoter.

20. The multicistronic vector of embodiment 19, wherein the inducible promoter is a tetracycline responsive promoter.

21. The multicistronic reporter vector of any one of embodiments 1-16 wherein the promoter is a tissue specific promoter.

22. The multispecific reporter vector of embodiment 21, wherein the tissue specific promoter is specific for cells of heart, blood, muscle, lung, liver, kidney, pancreas, brain, or skin.

23. The multicistronic reporter vector of any one of embodiments 1-22, further comprising a site-specific recombinase sequence located 3′ to the open reading frame.

24. The multicistronic reporter vector of embodiment 23, wherein the vector further comprises nucleic acid encoding a selectable marker, wherein the nucleic acid encoding the selectable marker is not operably linked to the promoter when the site-specific recombinase sequence has not recombined and is operably linked to the promoter when the site-specific recombinase sequence recombines with its target site-specific recombinase sequence.

25. The multicistronic reporter vector of embodiment 24, wherein the site-specific recombinase sequence is a FRT nucleic acid sequence and/or an attP nucleic acid and/or a loxP nucleic acid sequence.

26. The multicistronic reporter vector of embodiment 24 or 25, wherein the selectable marker confers resistance to hygromyocin, Zeocin™, puromycin, neomycin or an analog of hygromyocin, Zeocin™, puromycin, blasticidin or neomycin.

27. The multicistronic reporter vector of any one of embodiments 1-26, wherein nucleic acid encoding one or more polypeptides is inserted in-frame into the one or more MCS.

28. The multicistronic reporter vector or embodiment 27, wherein the one or more polypeptides comprise polypeptides that can be used to profile a single biological pathway, cross-talk between two or more biological pathways, synthetic lethality, cellular homeostasis, organelle homeostasis or a toxicity response.

29. The multicistronic reporter vector of any one of embodiments 1-28, further comprising one, two or three transcription units comprising a promoter and nucleic acid encoding a transgene located 5′ to the open reading frame comprising two or more cistrons, wherein the reporter vector further comprises a core insulator sequence and a polyA sequence located 3′ to the transcription units and 5′ to the open reading frame comprising two or more cistrons.

30. An acceptor cell for receiving a multicistronic reporter vector, wherein the acceptor cell comprises a recombinant nucleic acid integrated into a specific site in a host cell genome, wherein the recombinant nucleic acid comprises a first promoter operably linked to nucleic acid encoding a fusion polypeptide, wherein the fusion polypeptide comprises a reporter domain and a selectable marker domain, and wherein the nucleic acid comprises a site-specific recombinase nucleic acid sequence located at the 5′ end of the nucleic acid encoding the fusion polypeptide.

31. The acceptor cell of embodiment 30, wherein the promoter is a constitutive promoter.

32. The acceptor cell of embodiment 31, wherein the constitutive promoter is a CMV promoter, a TK promoter, an eF1-alpha promoter, a UbC promoter, a PGK promoter, a CAG promoter, an SV40 promoter, or a human β-actin promoter.

33. The acceptor cell of embodiment 32, wherein the promoter is an inducible promoter.

34. The acceptor cell of embodiment 33, wherein the inducible promoter is a tetracycline responsive promoter.

35. The acceptor cell of any one of embodiments 30-34, wherein the site-specific recombinase sequence is a FRT nucleic acid sequence and/or an attP nucleic acid sequence and/or a loxP nucleic acid sequence.

36. The acceptor cell of any one of embodiments 30-35, wherein the reporter domain of the fusion polypeptide is a fluorescent reporter domain.

37. The acceptor cell of any one of embodiments 30-36, wherein the fluorescent reporter domain is selected from GFP, EGFP, Emerald, Citrine, Venus, mOrange, mCherry, TagBFP, mTurquoise, Cerulean, UnaG, dsRed, eqFP611, Dronpa, RFP, TagRFPs, TdTomato, KFP, EosFP, Dendra, IrisFP, iRFPs, and smURFP.

38. The acceptor cell of any one of embodiments 30-37, wherein the reporter domain of the fusion polypeptide is an mCherry reporter domain.

39. The acceptor cell of any one of embodiments 30-38, wherein the selectable marker domain of the fusion polypeptide confers resistance to hygromycin, Zeocin™puromycin, blasticidin, neomycin or an analog of hygromycin, Zeocin™, puromycin, blasticidin, neomycin.

40. The acceptor cell of any one of embodiments 30-39, wherein the integrated recombinant nucleic acid further comprises nucleic acid encoding a tetracycline repressor polypeptide operably linked to a promoter.

41. The acceptor cell of embodiment 40, wherein the promoter is a human β-actin promoter or a CAG promoter.

42. The acceptor cell of any one of embodiments 30-41, wherein the recombinant nucleic acid is integrated in an adeno-associated virus S1 (AAVS1) locus, a chemokine (CC motif) receptor 5 (CCRS) locus, a human ortholog of the mouse ROSA26 locus, a hip11 (H11) locus or the citrate lyase beta like gene locus (CLYBL).

43. The acceptor cell of any one of embodiments 30-42, wherein the cell is an immortalized cell.

44. The acceptor cell of any one of embodiments 30-43, wherein the immortalized cell is a HEK293T cell, an A549 cell, an U2OS cell, an RPE cell, an NPC1 cell, a MCF7 cell, a HepG2 cell, a HaCat cell, a TK6 cell, an A375 cell or a HeLa cell.

45. The acceptor cell of any one of embodiments 30-44, wherein the cell is a pluripotent cell, an induced pluripotent stem cell, or a multipotent cell.

46. The acceptor cell of any one of embodiments 30-42, wherein the cell is a primary cell.

47. A method for generating an acceptor cell for receiving a multicistronic reporter vector, the method comprising introducing a recombinant nucleic acid to a cell wherein the recombinant nucleic acid comprises 5′ to 3′

a) a first nucleic acid for targeting homologous recombination to a specific site in the cell,

b) a first promoter,

c) site-specific recombinase nucleic acid,

d) nucleic acid encoding a first reporter polypeptide and a selectable marker,

e) a second nucleic acid for targeting homologous recombination to a specific site in the cell,

f) a second promoter and nucleic acid encoding a second reporter polypeptide,

wherein expression of the first reporter polypeptide without expression of the second reporter polypeptide indicates targeting integration of the recombinant nucleic acid to the specific site in the cellular genome and expression of the first and second reporter polypeptides indicates random integration in the cellular genome.

48. The method of embodiment 47, wherein the recombinant nucleic acid further comprises nucleic acid encoding a tetracycline repressor operably linked to a promoter 5′ to the second nucleic acid for targeting homologous recombination.

49. The method of embodiment 47 or 48 wherein the recombinant nucleic acid is integrated into the genome of the cell using:

a) an RNA guided recombination system comprising a nuclease and a guide RNA,

b) a TALEN endonuclease, or

c) a ZFN endonuclease.

50. The method of any one of embodiments 47-49, wherein cells expressing the first reporter polypeptide but not expressing the second reporter polypeptide are selected.

51. The method of any one of embodiments 47-50, wherein the site-specific recombinase nucleic acid is a FRT nucleic acid sequence and/or an attP nucleic acid sequence and/or a loxP nucleic acid sequence.

52. The method of any one of embodiments 47-51, wherein the first reporter polypeptide is fluorescent polypeptide and the second reporter polypeptide is a different fluorescent polypeptide.

53. The method of any one of embodiments 47-52, wherein the first and second reporter polypeptide is selected from GFP, EGFP, Emerald, Citrine, Venus, mOrange, mCherry, TagBFP, mTurquoise, Cerulean, UnaG, dsRed, eqFP611, Dronpa, RFP, TagRFPs, TdTomato, KFP, EosFP, Dendra, IrisFP, iRFPs and smURFP.

54. The method of any one of embodiments 47-53, wherein the first reporter polypeptide is an mCherry reporter and the second reporter polypeptide is GFP.

55. The method of any one of embodiments 47-54, wherein the selectable marker confers resistance to hygromycin, Zeocin™, puromycin, blasticidin, neomycin or an analog of hygromycin, Zeocin™, puromycin, blasticidin, neomycin.

56. The method of any one of embodiments 47-55, wherein the first promoter is a CMV promoter, a TK promoter, an eF1-alpha promoter, a UbC promoter, a PGK promoter, a CAG promoter, an SV40 promoter, or a human β-actin promoter and the second promoter is a CMV promoter, a TK promoter, an eF1-alpha promoter, a UbC promoter, a PGK promoter, a CAG promoter, an SV40 promoter, or a human β-actin promoter.

57. The method of any one of embodiments 47-56, wherein the first nucleic acid for targeting homologous recombination and the second nucleic acid for targeting homologous recombination target recombination to an AAVS1 locus, a CCRS locus, a human ortholog of the mouse ROSA26 locus, a H11 locus or a CLYBL locus.

58. The method of any one of embodiments 47-57, wherein the cell is an immortalized cell.

59. The method of any one of embodiments 47-58, wherein the immortalized cell is a HEK293T cell, an A549 cell, an U2OS cell, an RPE cell, an NPC1 cell, a MCF7 cell, a HepG2 cell, a HaCat cell, a TK6 cell, an A375 cell or a HeLa cell.

60. The method of any one of embodiments 47-59, wherein the cell is a pluripotent cell, an induced pluripotent stem cell, or a multipotent cell.

61. The method of embodiment 60, wherein the induced pluripotent stem cell is an WTC-11 cell or a NCRMS cell.

62. The method of any one of embodiments 47-58, wherein the cell is a primary cell.

63. An acceptor cell line generated with the method of any one of embodiments 47-62.

64. A multireporter cell comprising the acceptor cell of any one of embodiments 30-46 in which the multicistronic reporter vector of embodiments 27 or 28 has integrated into the genome of the acceptor cell.

65. The multireporter cell of embodiment 64, wherein the multicistronic reporter vector has integrated into AAVS1 locus of the acceptor cell.

66. A multireporter cell comprising a multicistronic reporter construct, wherein the multicistronic reporter construct comprises a promoter operably linked to an open reading frame, wherein the open reading frame comprises two or more cistrons; wherein each cistron comprises a nucleic acid encoding a different transgene product fused to a different reporter polypeptide, wherein expression of the open reading frame in a cell yields separate component polypeptide products from each cistron; and wherein expression of the transgene products is essentially stoichiometric.

67. The multireporter cell of embodiment 66, wherein the cistrons are separated from one another by nucleic acid encoding one or more self-cleaving peptide and/or one or more internal ribosome entry site (IRES).

68. The multireporter cell of embodiment 66 or 68, wherein each of the reporter polypeptides is a fluorescent reporter polypeptide.

69. The multireporter cell of any one of embodiments 66-68, wherein the fluorescent reporter domain is selected from GFP, EGFP, Emerald, Citrine, Venus, mOrange, mCherry, TagBFP, mTurquoise, Cerulean, UnaG, dsRed, eqFP611, Dronpa, RFP, TagRFPs, TdTomato, KFP, EosFP, Dendra, IrisFP, iRFPs, and smURFP.

70. The multireporter cell any one of embodiments 66-69, wherein the one or more self-cleaving peptides is one or more 2A peptides.

71. The multireporter cell of any one of embodiments 66-70, wherein the nucleic acid encoding the transgene product fused to the reporter polypeptide further comprises one or more nucleic acids encoding a peptide linker between the reporter polypeptide and a viral self-cleaving peptide.

72. The multireporter cell of embodiment 71, wherein the peptide linker comprises the sequence Gly- Ser-Gly.

73. The multireporter cell of any one of embodiments 66-72, wherein the multicistronic reporter vector comprises a promoter operably linked to an open reading frame, wherein the open reading frame comprises a first cistron, a second cistron, a third cistron and a fourth cistron, wherein each cistron comprises 5′ to 3′ nucleic acid encoding a transgene product fused to a fluorescent reporter polypeptide and nucleic acid encoding a linker peptide; wherein the first cistron and the second cistron are separated by nucleic acid encoding a first viral cleavage peptide, the second cistron and the third cistron are separated by nucleic acid encoding a second viral cleavage peptide the third cistron and the fourth cistron are separated by nucleic acid encoding an IRES.

74. The multireporter cell of any one of embodiments 66-73, wherein the mutlticistronic reporter vector further comprises one or more inducible elements located between the promoter and the open reading frame.

75. The multireporter cell of embodiment 74, wherein the inducible element is a Tet operator 2 (TetO2) inducible element.

76. The multireporter cell of any one of embodiments 66-75 wherein the promoter is a constitutive promoter.

77. The multireporter cell of embodiment 76 wherein the constitutive promoter is a CMV promoter, a TK promoter, an eF1-alpha promoter, a UbC promoter, a PGK promoter, a CAG promoter, an SV40 promoter, or a human β-actin promoter.

78. The multireporter cell of any one of embodiments 66-75 wherein the promoter is a tissue specific promoter.

79. The multireporter cell of embodiment 78, wherein the tissue specific promoter is specific for cells of heart, blood, muscle, lung, liver, kidney, pancreas, brain, or skin.

80. The multireporter cell of any one of embodiments 66-75, wherein the promoter is an inducible promoter.

81. The multireporter cell of embodiment 80, wherein the inducible promoter is a TRE promoter.

82. The multireporter cell of any one of embodiments 66-81, wherein the one or more transgene products comprise polypeptides that can be used to profile a single biological pathway, cross-talk between two or more biological pathways, cellular homeostasis, organelle homeostasis or a toxicity response. \

83. The multireporter cell of embodiment 82, where the profile is performed on a single cell.

84. The multireporter cell of any one of embodiments 64-83, wherein the reporter polypeptide can be visualized by microscopy, high throughput microscopy, fluorescence-activated cell sorting (FACS), lumininesence, or using a plate reader.

85. The multireporter cell of any one of embodiments 66-84, further comprising one, two or three transcription units comprising a promoter and nucleic acid encoding a transgene located 5′ to the open reading frame comprising two or more cistrons, wherein the reporter vector further comprises a core insulator sequence located 3′ to the transcription units and 5′ to the open reading frame comprising two or more cistrons.

86. A method for generating a multireporter cell, the method comprising introducing the multicistronic reporter vector of embodiment 27 or 28 into the acceptor cell of any one of embodiments 33-49.

87. The method of embodiment 86, wherein the recombinase associated nucleic acid sequence is FRT nucleic acid sequence and the acceptor cell comprises a flp recombinase.

88. The method of embodiment 87, wherein the recombinase associated nucleic acid is attP and the acceptor cell comprises a Bxbl recombinase, a PhiC31 recombinase, or R4 recombinase.

89. The method of embodiment 87, wherein the recombinase associated nucleic acid sequence is loxP nucleic acid sequence and the acceptor cell comprises a CRE recombinase.

90. A library of multireporter vectors, wherein the library comprises multicistronic reporter vectors comprise nucleic acid encoding different transgene products fused to reporter polypeptides of any one of embodiments 1-29, or wherein the library comprises a plurality of reporter cells of any one of embodiments 64-85, wherein two or more of the different transgene products on each vector are expressed essentially stoichiometrically when introduced to cells.

91. The library of embodiment 90, wherein the reporter vectors encode two or more transgenes encode polypeptides that can be used to profile a single biological pathway, cross-talk between two or more biological pathways, cellular homeostasis, organelle homeostasis or a toxicity response.

92. The library of embodiment 90 or 91, wherein the biological pathway is a pathway associated with a disease.

93. The library of embodiment 92, wherein the disease is cancer, a cardiovascular disease, a neurodegenerative disease or an autoimmune disease.

94. The library of embodiment 92 or 93, wherein the biological pathway is a pathway associated with toxic response mechanism within the cell.

95. The library of embodiment 92 or 93, wherein the biological pathway is a pathway associated with cell proliferation, cell differentiation, cell death, apoptosis, autophagy, DNA damage and repair, oxidative stress, chromatin/epigenetics, MAPK signaling, PI3K/Akt signaling, translational control, cell cycle and checkpoint control, cellular metabolism, development and differentiation signaling, immunology and inflammation signaling, tyrosine kinase signaling, vesicle trafficking, cytoskeletal regulation or ubiquitin pathway.

96. The library of any one of embodiments 90-95, wherein each multicistronic vector comprising transgenes used to profile a specific single biological pathway, specific cross-talk between two or more biological pathways, synthetic lethality, a specific cellular homeostasis, a specific organelle homeostasis, a specific toxicity response, a specific tissue comprises a common transgene product fused to a different reporter polypeptide for each multicistronic reporter vector.

97. The library of any one of embodiments 90-96, wherein each multicistronic vector comprising transgenes used to profile a specific single biological pathway, specific cross-talk between two or more biological pathways, a specific cellular homeostasis, a specific organelle homeostasis or a specific toxicity response comprises a common transgene product fused to reporter polypeptide.

98. A library of acceptor cells for receiving multicistronic reporter vectors, wherein the library comprises acceptor cells according to embodiments 30-46.

99. A library of multireporter cells, wherein each cell in the library comprises multicistronic reporter vector comprising nucleic acids encoding different transgene products fused to reporter polypeptides, wherein the different nucleic acids encoding different transgene products on each vector are expressed essentially stoichiometrically when introduced to cells.

100. The library of embodiment 98 or 99, wherein the library comprises different immortalized cells.

101. The library of embodiment 100, wherein the library includes one or more of a HEK293T cell, an A549 cell, an U2OS cell, an RPE cell, an NPC1 cell, a MCF7 cell, a HepG2 cell, a HaCat cell, a TK6 cell, an A375 cell or a HeLa cell.

102. The library of embodiment 98 or 99, wherein the library comprises different pluripotent, multipotent and/or progenitor cells.

103. The library of embodiment 102, wherein the different pluripotent or multipotent cells include one or more of an induced pluripotent stem cell, a multipotent cell, a hematopoietic cell, an endothelial progenitor acceptor cell, a mesenchymal progenitor cell, a neural progenitor cell, an osteochondral progenitor cell, a lymphoid progenitor cell or a pancreatic progenitor cell.

104. The library of embodiment 98 or 99, wherein the library of pluripotent or multipotent cells multireporter cells are differentiated after introduction of the multicistronic reporter vector.

105. The library of embodiment 98 or 99, wherein the library comprises different primary cells.

106. The library of embodiment 105, wherein the primary cells comprise one or more of a cardiomyocyte, a muscle cell, a lung cell, a liver cell, a kidney cell, a pancreatic cell, a neuron, or a tumor cell.

107. The library of any one of embodiments 99-106, wherein each cell in the library comprises the same multicistronic reporter vector.

108. The library of cells of any one of embodiments 99-107, wherein cells in the library comprise different multicistronic reporter vectors.

109. The library of cells of embodiment 108, wherein different multicistronic reporter vectors were introduced to isogenic acceptor cells.

110. The library of cells any one of embodiments 99-109, wherein the reporter vectors encode one or more transgenes one or more polypeptides comprise polypeptides that can be used to profile a single biological pathway, cross-talk between two or more biological pathways, cellular homeostasis, or phenotypic features.

111. The library of embodiment 110, wherein the biological pathway is a pathway associated with a disease.

112. The library of embodiment 111, wherein the disease is cancer, a cardiovascular disease, a neurodegenerative disease or an autoimmune disease.

113. The library of any one of embodiments 110, wherein the biological pathway is a pathway associated with toxic response mechanism within the cell.

114. The library of any one of embodiments 110-113, wherein the biological pathway is a pathway associated with cell proliferation, cell differentiation, cell death, apoptosis, autophagy, DNA damage and repair, oxidative stress, chromatin/epigenetics, MAPK signaling, PI3K/Akt signaling, translational control, cell cycle and checkpoint control, cellular metabolism, development and differentiation signaling, immunology and inflammation signaling, tyrosine kinase signaling, vesicle trafficking, cytoskeletal regulation or ubiquitin pathway.

115. The library of any one of embodiments 99-114, wherein each multicistronic vector comprising transgenes used to profile a specific single biological pathway, specific cross-talk between two or more biological pathways, synthetic lethality, a specific cellular homeostasis, a specific organelle homeostasis, a specific toxicity response, a specific tissue, or a specific phenotypic feature comprises a common transgene encoding a polypeptide fused to a different reporter polypeptide.

116. The library of any one of embodiments 99-115, wherein each multicistronic vector comprising transgenes used to profile a specific single biological pathway, specific cross-talk between two or more biological pathways, a specific cellular homeostasis, a specific organelle homeostasis , a specific toxicity response, or a specific phenotypic feature comprises a common transgene encoding a polypeptide fused to reporter polypeptide.

117. A kit comprising one or more multicistronic reporter vectors of any one of embodiments 1-29.

118. A kit comprising one or more acceptor cells of any one of embodiments 30-46.

119. A kit comprising one or more multicistronic reporter vectors of any one of embodiments 1-29 and one or more acceptor cells of any one of embodiments 30-46.

120. A kit comprising one or more multireporter cells of any one of embodiments 64-85.

121. The kit of any one of embodiments 118-120, wherein the kit comprises a library of acceptor cells and/or reporter cells arrayed in a multiwell plate.

122. The kit of embodiment 121, wherein the cells in the multiwell plate are cryopreserved.

123. A method of profiling two or more polypeptides in a live cell, the method comprising determining the expression of the two or more of the transgenes and/or location of the two or more transgene products of a multireporter cell of any one of embodiments 64-85 or a cell comprising the multicistronic vector of any one of embodiments 1-29.

124. The method of embodiment 123, wherein the method is used to profile a single biological pathway, cross-talk between two or more biological pathways, synthetic lethality, cellular homeostasis, organelle homeostasis or a toxicity response.

125. The method of embodiment 123 or 124 wherein determining the expression of the two or more of the transgenes and/or location of the two or more transgene products is determined at one or more time points.

126. The method of embodiment 125, wherein determining the expression of the two or more of the transgenes and/or location of the two or more transgene products is determined at one or more of 1 minute, 5 minutes, 10 minutes, 15 minutes, 30 minutes, 45 minutes, 1 hour, 2 hours, 3 hours, 4 hours, 5 hours, 6 hours, 12 hours, 24 hours, 2 days, 4 days, 7 days, 14 days, 21 days, 30 days, 1 month, 3 month, 6 month, 9 month, 1 year, or more than 1 year.

127. The method of any one of embodiments 123-126, wherein the cells are prepared by the method of any one of embodiments 86-89.

128. The method of any one of embodiments 123-126, wherein the cells are a library of cells according to embodiments 90-116.

129. The method of embodiment 129, wherein the cells are derived from an isogenic acceptor cell.

130. The method of embodiment 128 or 129, wherein the cells or the library are pooled prior to analysis.

131. A method of measuring the effects of an agent on the profile of two or more polypeptides in a live cell, the method comprising subjecting a multireporter cell of any one of embodiments 64-85 to the agent and determining the expression of the two or more of the transgenes and/or location of the two or more transgene products in the cell in response to the agent.

132. The method of embodiment 131 wherein the agent is a drug or drug candidate.

133. The method of embodiment 131 or 132, wherein the method is a toxicology screen.

134. The method of any one of embodiments 131-133, wherein determining the expression of the two or more of the transgenes and/or location of the two or more transgene products is performed in a library of multireporter cells.

135. The method of any one of embodiments 131-134, wherein the profile is obtained using a single cell.

136. The method of any one of embodiments 131-135, wherein the expression of the two or more of the transgenes and/or location of the two or more transgene products is measured by microscopy, high throughput microscopy, fluorescence- activated cell sorting (FACS), lumininesence, using a plate reader, mass spectrometry, or deep sequencing.

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

Further details of the invention are illustrated by the following non-limiting Examples. The disclosures of all references in the specification are expressly incorporated herein by reference.

### EXAMPLES

### Example 1

### Development of Monoclonal Acceptor Cells

Genome editing tools were optimized to generate monoclonal cell lines containing an ‘acceptor site’. Using an acceptor site enables reliable, rapid, and consistent site-specific integration of reporter constructs of choice. The acceptor site (FIG. 1A, lower panel) comprisesl) two AAVS1 sequences to direct integration of the acceptor site to the AAV locus; 2) a fluorescent marker visible by microscopy (mCherry); 3) an antibiotic (Zeocin - Thermo Fisher) resistance selection marker ; 4) a FRT site used for site-specific recombination of the reporter construct by flippase (F1p) ; 5) a constitutive promoter, SV40, that drives Zeocin (Thermo Scientific™) resistance gene and mCherry expression, enabling antibiotic selection and fluorescence screening, respectively, to identify positive acceptor cell clones; 6) human b-actin promoter driving the Tetracycline Repressor proteins (TetR).

The acceptor site was stably integrated into the genome of cells of interest using Cas9-mediated genome editing protocols. The AAVS1 locus was used as the integration site in accordance with design of the acceptor site. The acceptor site was synthesized and cloned into an AAVS1-donor vector (GeneCopoeia™) and co-transfected with Cas9:sgRNA(AAVS1), a guide RNA (gRNA), complementary to the AAVS1 sequence of interest, resulting in stable integration at a single integration site (IS), namely the AAVS1 locus located between exons 1 and 2 of the PPP1R12C locus of chromosome 19. Transfection and junction PCR combined with Southern blot analysis were used to confirm correct acceptor site integration in acceptor cell clones obtained after selection.

The acceptor site was transfected into HEK 293T U2OS, A549, HeLa, RPE and NPC1 cells plated in 2-4 96-well plates. HEK 293T,U2OS, A549 and Hela cells were successfully transfected with efficiencies above 60% (as estimated by the number of mCherry positive cells) using Lipofectamine 3000 (Life Technologies™) and following the protocol suggested by the manufacturer. RPE and NPC1 cell lines showed a very low (<2%) transfection efficiency using Lipofectamine and the same protocol.

Transfection of RPE and NPC1 cell lines by electroporation was also attempted using the Neon® Transfection System (Thermo Scientific™) with 4 different conditions of varying cell density, construct concentration and applied voltage. One of the test conditions (1350V, 20 ms pulse, two pulses, with a 10 μl tip) resulted in high transfection efficiency (>60%) of RPE cells, however transfection of the NPC1 line was unsuccessful.

After enriching for U2OS, HEK293T,RPE, A549 and HeLa clones with acceptor site integration using Zeocin selection, single colonies of each cell line were generated and screened for integration at the AAVS1 locus by PCR and random integration; and site-specific single/double allele integration by Southern blot. Clones that were mCherry positive and Zeocin resistant were plated in 3-5 96 well plates with 1-2 cells/well and screened using junction PCR. About 58%-91% of clones were confirmed to have site-specific integration as assessed by junction PCR (Table 1).

TABLE 1

| | | | | N clones | | N clones | | |
|---|---|---|---|---|---|---|---|---|
| | N 96 | | % PCR- | screened for | % | screened for | % clones | overall % |
| Cell | well | N | positive | single allele | clones | random | without | acceptor |
| Line | plates | clones | clones | integration (SI) | with SI | integration (RI) | RI | cell lines |
| U2OS | 3 | 38 | 58% | 10 | 40% | 6 | 0% | 0 |
| 293T | 2 | 19 | 79% | 13 | 23% | 6 | 17% | 0 |
| RPE | 4 | 54 | 91% | 9 | 67% | 5 | 100% | 56 |
| A549 | 2 | 21 | 90% | 13 | 69%% | 12 | 8% | 5 |
| Hela | 2 | 13 | 85% | 7 | 71% | 11 | 27% | 8 |

Two probes for Southern blotting were designed: 5′ probe (FIG. 1A top panel) to confirm single allele integration of the acceptor site at the AAVS1 locus; and internal probe (FIG. 1A bottom panel) to confirm single copy integration without random integration in the genome. After digestion of genomic DNA with EcoRI, the 5′ probe detects a single band at 2.8 kb in the case of integration in both alleles or a single band at 8.2 kb in the case of no integration. Samples with single allele integration had 2 bands due to integration in one allele (2.8 kb) and no integration in the other (8.2 kb) (FIG. 1C). A non-radioactive Southern blot protocol was determined to be insufficiently sensitive for screening. For each cell line at least 9 PCR positive clones were selected to be screened with 5′ probe. Genomic DNA of clones confirmed to have single allele integration with the 5′ probe was then probed with the internal probe to identify clones without random integration of the acceptor site in other genomic loci identified by the presence of a single band at 10 Kb (FIG. 1D).

Site-specific acceptor site in the absence of random integration was achieved in RPE cells with an efficiency of 56%. For A549 and Hela a total of 21 and 13 clones were screened by PCR and southern blotting and only 1 clone had a single copy integration of the acceptor site at the AAVS1 locus, corresponding to an overall recombination efficiency of 5 and 8% respectively. However, Southern blot screening of 9 U2OS and 13 HEK293T clones failed to identify any clones with single copy integration of the acceptor site at the correct locus (Table 1).

### Example 2

### Development of a Multicistronic Platform

A tetracistronic reporter was designed to incorporate a constitutive promoter (CMV) that drives the expression of a single open reading frame (ORF) containing three multiple cloning sites (MCSs) separated by 2 unique viral 2A self-cleaving peptides and a fourth MCS separated by an internal ribosome entry site (IRES) element that allows for translation initiation in the middle of the mRNA sequence (FIG. 2). The 2A self-cleaving peptides allow multiple proteins to be encoded as polyproteins, which dissociate into component proteins upon translation. The 2A peptide sequence impairs normal peptide bond formation through a mechanism of ribosomal skipping. From the family of peptide 2A cleavage sequences the P2A and T2A versions were identified as optimal candidates since these have been shown to exhibit most efficient cleavage (Kim et al, PLOS One, 2011). To increase cleavage efficiency of the viral peptide a Gly-Ser-Gly linker was added between the protein N-terminal and the 2A-peptide sequences (FIG. 2). Immediately downstream of the promoter there are 2 copies of the Tet operator 2 (TetO2) to which the TetR (expressed from the acceptor site) binds inhibiting expression of the genes of interest. Gene expression is induced upon addition of doxycycline (a tetracycline analog). Furthermore, the FRT sequence was fused to a promoterless hygromycin-resistance gene that can only be expressed when correct gene targeting occurs. The reporter constructs are assembled using InFusion. After cloning the vectors are verified by Sanger sequence. Once the reporter is completed the sequence is again verified to make sure the 3 or 4 reporters are inserted.

To test the reporter localization or activation, cells are transiently transfected and the localization is assessed by microscopy or if it's a biosensor the cells are treated with a compound that activates the corresponding pathway to verify that the biosensor is responsive.

Certain proteins that are perturbed by the presence of extra amino acids in the C′ cannot be placed on the 1st or 2nd position because the 2A cleavage leaves extra amino acids on the C′ of the proteins. The synthesized nucleotide sequence was cloned into a pCDNA5/FRT vector backbone. The FRT target site was linked to a mCherry reporter gene to allow fluorescence screening. This vector was designed to recombine into the acceptor cell lines generated when co-transfected with a second vector harboring the Flippase (Flp) (pOG44, Thermo Fisher Scientific).

The multicistronic vector was co-transfected with pOG44 into acceptor cell lines and transfection conditions were optimized for production of multicolor stable reporter cell lines that may be used as a basis for assay development and disease modeling. The recombination rate of the flippase (Flp) recombinase employed is of one in 10-6 cells (O'Gorman, S., Fox, D. T. & Wahl, G. M. Science 251, 1351-1355 (1991)).

Upon generation of a stable reporter cell lines, the effect of position within the multicistronic vector was evaluated. Multiple rounds of optimization were used to determine an optimal construct that would yield stable balanced expression of functional fluorescently tagged proteins of interest in acceptor cell lines. Such cell lines would be a useful foundation for live cell microscopy-based assays.

### Example 3

### Multiparametric Visualization of RAS-MAPK Signal Transduction in Live Cells

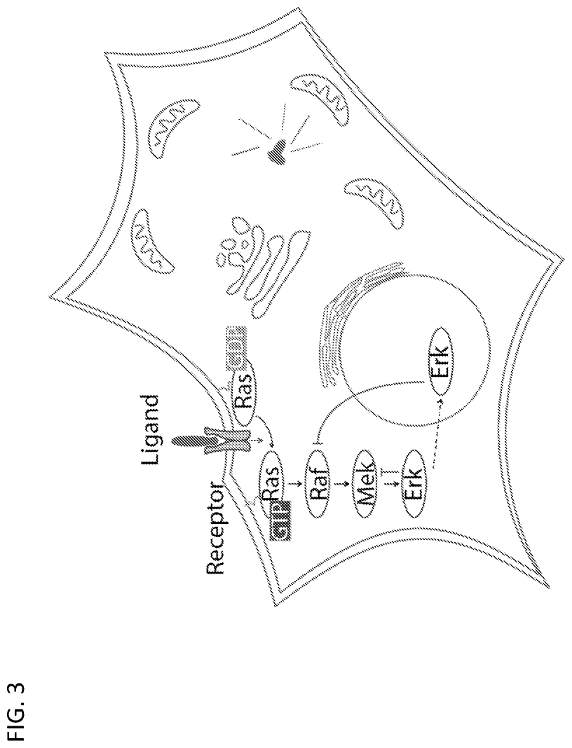

The spatiotemporal modulation of Ras/MAPK pathway signaling proteins in the cell (FIG. 3) is an important facet of their signaling behavior. Ras/MAPK signaling proteins localize in different cellular compartments, depending on pathway activation status. New assays that comprehensively probe the dynamic activity of the entire pathway in intact cells can assist in the identification and optimization of next-generation inhibitors that circumvent resistance mechanisms. Described herein is a novel approach to monitor the cellular activity of putative inhibitors in high-throughput compatible multiplexed assays that report the dynamic activity of the entire Ras/MAPK pathway in live cells. To monitor the pathway live, stable cell lines with balanced expression of fluorescently tagged proteins were used.

To generate two color cell lines, CRaf and mutant KRas(G12C) constructs with N-terminal fluorescent protein tags were cloned into a tetracycline-inducible bicistronic expression vector. A stable cell line was generated by co-transfecting Flp-In TREx 293 cells with the bicistronic vector and Flp-recombinase expression plasmid using FuGENE6® (Promega™) followed by selection with hygromycin B.

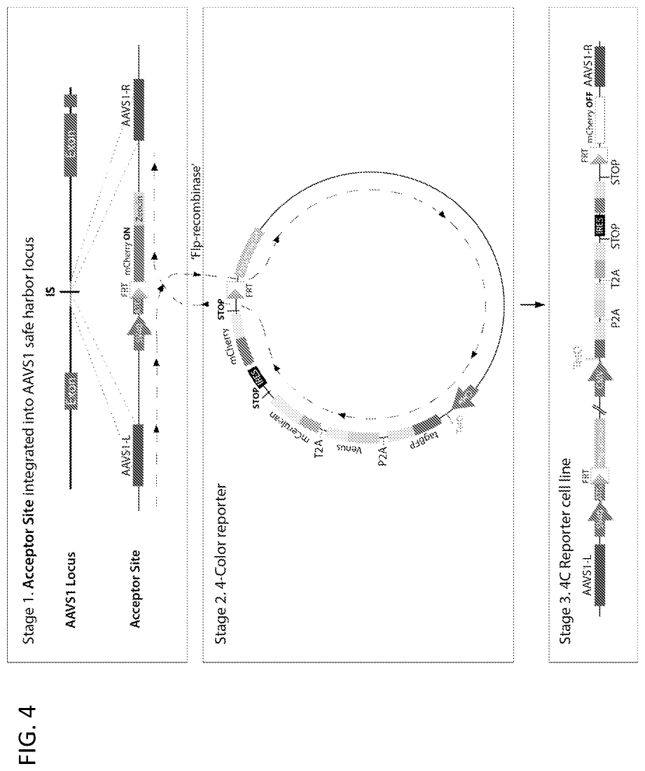

The two-color stable cell lines were plated in 96 well glass-bottom plates overnight and treated with doxycycline at 1 μg/m1 to induce fluorescent reporter expression. Cells were treated with different compounds for four or nine hours. Cells were imaged with an epifluorescence Nikon™microscope with an OKOLab™ incubator system and images were acquired using a Plan Fluor™40× objective and NIS-Elements® software (Nikon™). An analogous procedure, using N-terminal tagged KRas(G12C), CRaf, Mek and Erk cloned into the multicistronic construct, was used to generate monoclonal, inducible 4-color cell lines with approximately balanced reporter construct expression level (FIG. 4 and FIG. 5, control)

The 4-color lines were used to visualize the MAPK pathway in live cells. HEK293 multicolor stable cell line expressing KRas(G12C), C-Raf, Mek and Erk reporters show correct localization of the 4 proteins at the plasma membrane, plasma membrane and cytoplasm, cytoplasm, nucleus, respectively (FIG. 5). Monitoring of cells under the microscope allowed visualization of the response to Ras-MAPK pathway inhibitors. To monitor response to inhibitors, after protein induction cell were treated with: GDC-0879, FR180204, lovastatin and PD0325901 for four or nine hours. Representative images of the different treatments are shown in FIG. 5.

GDC-0879 is an ATP-mimetic Raf inhibitor that binds to the active CRaf conformation and induces CRaf plasma membrane targeting. Lovastatin is an antagonist of HMG-CoA which blocks the processing and membrane localization of Ras proteins via inhibition of prenylation. FR180204 is an Erk inhibitor that prevents its catalytic activity but not its phosphorylation. Treatment with GDC-0879, a Raf inhibitor, showed clear enrichment of CRaf at the plasma membrane upon compound treatment while the other proteins remained in the same location relative to the control. Treatment with FR180204, an Erk inhibitor showed moderate increase of CRaf levels at the plasma membrane. This can be explained due to the release of negative feedback that is triggered by active Erk, acting directly on C-Raf. Treatment with Lovastatin which affects KRas inactivation results in the absence of KRas at the membrane, concomitantly C-Raf is not enriched at the membrane and the downstream effector of MAPK pathway, mCherry-Erk, changes its localization to the cytoplasm. Treatment with PD0325901a Mek inhibitor results in redistribution of the downstream effector, Erk, from the nucleus to the cytoplasm, indicating that Erk is not active. As expected, none of Mek upstream proteins (KRas and C-Raf) showed alteration in localization.

Lovastatin causes a significant displacement of KRas from the cell membrane (***p<0.0001). TA-155 treatment causes a similar displacement of KRas from the cell membrane (***p<0.0001) validating this molecule as an effector of the pathway.

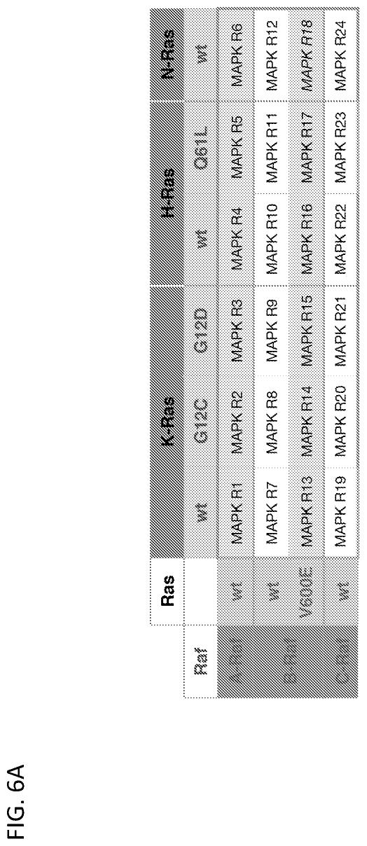

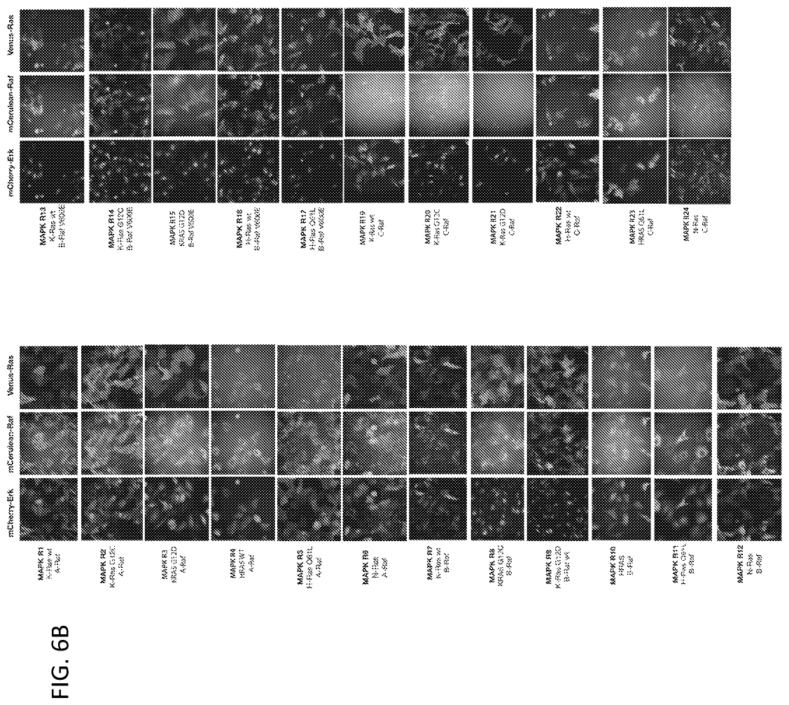

To further show the utility of the multiplex assay a library with 23 different reporter cells was generated using a library of 24 MAPK vectors (FIG. 6A). The MAPK library is composed of a collection of multicistronic construct encompassing 4 proteins: 3 fluorescently labeled - mcherry-Erk, mCerulean-Raf (isoforms and mutants), Venus-Ras (isoforms and mutants) and additionally untagged Mek. Untagged Mek and mCherry-Erk are fixed in all constructs. Each Ras and Raf wildtype, or isoforms (K-, H-, N-Ras and A-, B-, C-Raf) and mutants (K-RasG^{G12 C}, K-R_{G12D}, H-Ras^{Q61L} and B-Raf^{V600E})were combined in the MAPK constructs. MAPK reporter constructs are denoted “MAPK R” and numbered from “1-24”. A collection of 24 vectors was recombined in U2OS^{A-Tet} acceptor cell line with Bxb1 to generate reporter cell lines using FuGENE6® (Promega™). Cells were selected for 3 days with 75ug/ml of hygromycin B. After selection cells were expanded and validated by testing tetracycline induction and plated in 96 glass bottom plates for imaging to verify presence of the 3 fluorescently labeled proteins. All reporter cell lines were treated with doxycycline at 1 ug/ml for 12-16 hours to induce fluorescent reporter expression. Cells were imaged with an epifluorescence Nikon™ microscope with an OKOLab™ incubator system and images were acquired using a Plan Fluor' 40x objective and NIS-Elements® software (Nikon™) (FIG. 6B).

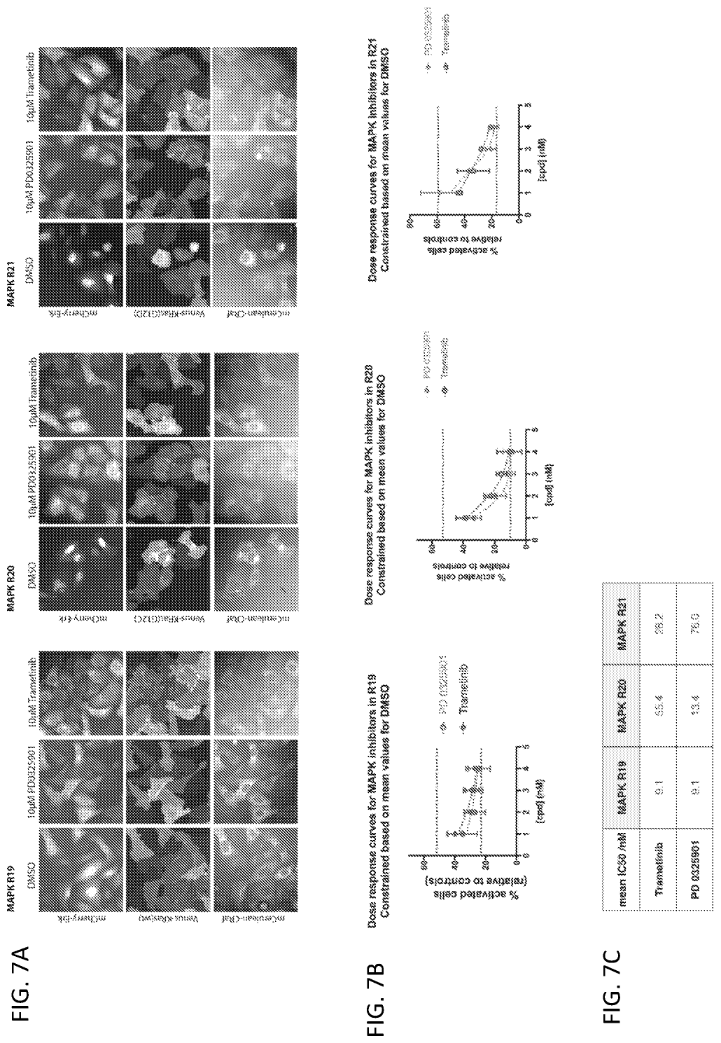

Three MAPK reporter cell lines were used to visualize the MAPK pathway in live cells and to analyze dose responses of MAPK inhibitors. U2OS MAPK R19, R20 and R21 multicolor stable cell line expressing mCerulean-CRaf, untagged Mek, mCherry-Erk and Venus-KRas (wt, G12C and G12D, respectively) were plated in glass bottom well 96 well plates. Twenty-four hours after plating cells were treated with doxycycline at 1 μg/ml for 5 h to induce fluorescent reporter expression. Monitoring of cells under the microscope allowed visualization of the response to Ras-MAPK pathway inhibitors. Two inhibitors, Trametinib an inhibitor of Mekl and PD 0329501, inhibitor of Mek1/2 were used in the assay. Trametinib is a reversible, allosteric inhibitor of MEK1/2 that binds to Serinel2 in the activation loop. The inhibitors were tested in duplicates in four ten-fold serial dilutions starting at 10 μM with data collection 1 hour after compound addition. Images were acquired in a Nikon Ti-E inverted Microscope equipped with motorized stage with autofocus and OkoLab stage top environmental control chamber (supplied with CO2 and temperature controlled for live cell imaging). 4 positions per well were acquired with a 20× plan-apo objective using CFP, YFP, TexasRed and Dapi filter sets (FIG. 7B)

The subcellular localization of mCherry-Erk was used as a metric for MAPK pathway activation; cells with active MAPK signaling show nuclear localization of Erk, whereas cells with inactive or inhibited MAPK signaling show nuclear exclusion of Erk. For each assay, the images were analyzed using CellProfiler (open source software for image analysis, Kamensty, L. et al., Bioinformatics (2011)) for quantification of the ratio of nuclear versus cytoplasmic Erk fluorescence intensity and the data generated was further processed to determine compound IC_{50S}.

Image analysis used the open source software CellProfiler. After importing the images, nuclei were identified from the Hoescht image as primary objects. The cytoplasm for each cell was identified from the K-Ras image as secondary objects using propagation from the primary object. The nuclear area of each cell was subtracted from the cytoplasm area to give the final cytoplasm mask. mCherry intensity for the nucleus and the cytoplasm of each cell was calculated from the original mCherry-Erk image and the nuclear to cytoplasm ratio of mCherry intensity was calculated per cell and exported as a HDFS file for analysis. Data from CellProfiler analysis was exported to Python programming language (Python Software Foundation, world wide web at python.org) for further computation.

To quantify the degree of inhibition (mCherry-Erk cytoplasm localization) of each compound we used Gaussian kernel density estimation using the SciPy library of Python open-source software. The untreated (vehicle) and maximally inhibited (highest concentration of most potent inhibitor) control values per cell line were used to generate density plots of the distribution of values. The data from individual wells was plotted against the control density plots and the probability of each cell to be classified as inhibited or not inhibited was used to determine the percentage of inhibition of each compound at 4 different concentrations. The values were exported to GraphPad Prism 7.0 software (LaJolla, Calif., USA) and we used a non-linear regression to fit the data and calculate the concentration where we observe 50% activity of the inhibitor (IC₅₀) (FIGS. 7B and 7C).

Trametinib activity was detected in all 3 cell lines (FIG. 7A) with IC_{50S} value of 9 nM for cells expressing K-Ras(wt) and 55 and 28 nM for cells expressing K-Ras(G12C) and K-Ras(G12D), respectively. The mutant cell lines present higher IC_{50S} (FIG. 7C).

PD 0329501 activity was detected in all 3 cell lines (FIG. 7A) with IC_{50S} value of 9 nM for cells expressing K-Ras(wt) and 13 and 76 nM for cells expressing K-Ras(G12C) and K-Ras(G12D), respectively. The mutant cell lines present higher IC_{50S} (FIG. 7C).

These results demonstrate that the multiplex assay allows one to monitor, measure and compare MAPK inhibitor activity, dose response and determine IC_{50S} for different KRas backgrounds.

### Example 4

### Optimization of Acceptor Site

The acceptor site was further optimized to be more easily adaptable and allow for more efficient differentiation between targeted and untargeted integration. As illustrated in FIG. 1E, the updated acceptor site includes 1) a CMV driven GFP element and 2) an attP site for recombination using Bxbl recombinase in addition to the FRT site. By redesigning the acceptor site to contain GFP driven by the CMV promoter (CMV-GFP) after the AAVS1-right homology recombination arm (FIG. 1E), it is easier to distinguish between targeted integrations at the AAVS1 locus or random integrations in the genome. Cells with a randomly integrated redesigned acceptor site will fluoresce green. Cells with an integrated redesigned acceptor site-either targeted or randomly integrated—will fluoresce red due to expression of mCherry. Thus, cells with targeted integration will be exclusively red, while cells with any random integration will also express GFP and be red and green. This second approach allows for the use of fluorescence microscopy to identify cells without random integration or FACS to sort cells without recombination, and without having to conduct a southern blot screen. (FIG. 1F). The attP site enables site-specific recombination in the acceptor site.

The new acceptor site was stably integrated into an integration site within the genome of cells of interest using established Cas9-mediated genome editing protocols. As for the acceptor site design, the safe harbor AAVS1 locus was used as the integration site in accordance with design of the acceptor site. The acceptor site was synthesized and cloned into an AAVS1-Donor vector (GeneCopoeia) and co-transfected with Cas9:sgRNA(AAVS1), a guide RNA (gRNA), complementary to the AAVS1 sequence of interest, resulting in stable integration at a single integration site (IS), namely the AAVS1 locus located between exons 1 and 2 of the PPP1R12C locus of chromosome 19 . Transfection and stable integration were done using a RNA-guided Cas9-CRISPR-mediated genome editing followed by antibiotic selection with Zeocin. After clonal cell growth, 2 junction PCR (FIG. 1A) were used to identify single allele integration of the acceptor site. A primer pair was designed to detect a single PCR product band at the insertion site (IS). A second pair of primers amplifies a PCR product from alleles where integration occurred. Clonal cells with single allele integration should present amplification for both PCRs. Next, to determine copy number integration at the correct site droplet digital PCR (ddPCR) was conducted with a specific probe for mCherry gene. A probe for the housekeeping gene RPP30 with 2 copies in the human genome, was used to quantify the relative copy number in the samples (Table 2).

TABLE 2

| | | N clones | | | |
|---|---|---|---|---|---|
| | | integration | N clones | N clones | % of clones |
| | N | at the | single | with | with single- |
| | clones | AAVS1 | allele | 1 copy | copy at the |
| | screened | locus | integration | (ddPCR) | AAVS1 locus |
| HepG2 | 43 | 35 | 34 | 11 | 26% |
| U2OS | 61 | 52 | 43 | 10 | 16% |
| A549 | 20 | 19 | 12 | 2 | 10% |
| A375 | 73 | 73 | 72 | 10 | 14% |

Site-specific, single copy, single allele integration of the optimized acceptor site in the absence of random integration was achieved in HepG2, U2OS, A549 and A375 cells with an efficiency of 26, 16, 10 and 14%, respectively. Forty-three clones of HepG2 were screened by PCR and ddPCR; from those 11 had a single copy integration in the correct locus. A total of 61 U2OS clones were screened by PCR and ddPCR and 10 had a single integration at the AAVs1 locus. For A549 cells a total of 20 clones were screened by PCR and ddPCR and 2 clones had a single copy integration of the acceptor site at the AAV locus, corresponding to an overall integration efficiency of 10%. For A375 cells a total of 73 clones were screened and 10 had a single copy integration at the AAVS1 locus. In the case of U2OS and A549 the screen previously conducted by PCR and Southern blot failed to identify any colonies a with single copy integration of the acceptor site at the correct locus. The optimized acceptor cell platform is thus much more efficient for generation of acceptor cells.

Five quality control steps were implemented to select for acceptor cell lines that have an integration site intact and fully functional with best performances (Table 3). The first step is sequencing the genomic DNA of the acceptor cell lines at 5′ and 3′ site of insertion in AAVS1 genomic locus to confirm that the recombination process did not trigger any small insertion, deletion or mutation flanking the recombination site. Second step is evaluating the homogeneity of the acceptor cells to ensure that they come from a single cell—for that flow cytometry analysis is conducted and the coefficient of variation (CV) of the mCherry fluorophore is calculated (Step 2—CV(mCherry)=51%). To determine if the acceptor cells contain a functional Tetracycline regulation, the cells are co-transfected with B×B1 and a dual color multicistronic construct that contains MTS-Venus, H2B-TagBFP under tetracycline regulation. Reporter cells are selected with hygromycin B for 3 days and left to grow for 6-10 days. The number of colonies formed is scored (Step 4). After selection, the cells are treated with 1 μg/ml doxycycline for 12-15 h. The next validation and QC steps are conducted using flow cytometry analysis. For Step 3 induced cells are compared to uninduced cells to verify loss of mCherry and gain of Venus and TagBFP fluorescence. To determine if the variability of the protein expression in the polyclonal population after reporter integration is similar to that of the acceptor cell line population, the CV of the fluorescence intensity of each of the fluorophores present in the multicistronic vector is determined (Step 4—CV(TagBFP)=67% and CV(Venus)=69%) and compared to the parental CV. To verify if the expression of the different proteins placed on the acceptor site is homogenous and that the payload is intact we determined the coefficient of correlation (r) based on the flow cytometry values of fluorescence intensity of the proteins expressed (coefficient of correlation r=0.89, Step 5).

TABLE 3

| | | Method of | |
|---|---|---|---|
| | Goal | Evaluation | Characteristics |
| Step | Sequencing of insertion | gDNA | Correct |
| 1 | site | sequencing | |
| Step | Aceeptor cell line | FACS | mCherry CV = 50.8% |
| 2 | homogeneity | | (±2.1) |
| Step | Testing tetracycline | Microscopy | Yes |
| 3 | regulation | | |
| Step | 1. Quantify cell line | FACS | 1. CV TagBFP = 67% |
| 4 | expression homogeneity | | CV Venus = 69% |
| | post-recombination | | |
| | 2. Number of stable | | 2. many |
| | colonies | | |
| Step | Compare expression levels | FACS | correlation expression |
| 5 | of different reporters | | levels: r² = 0.89 |

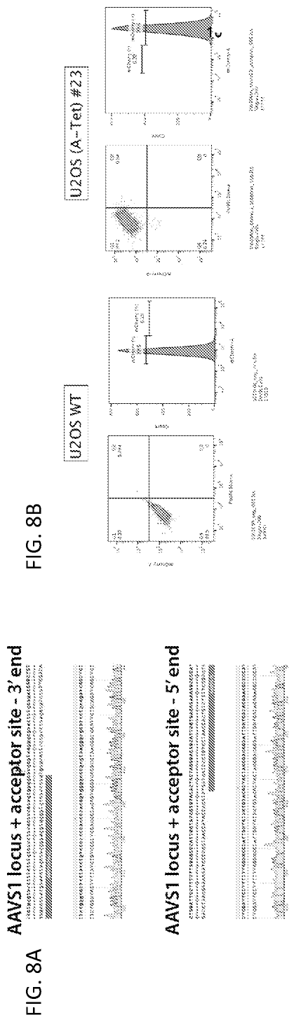

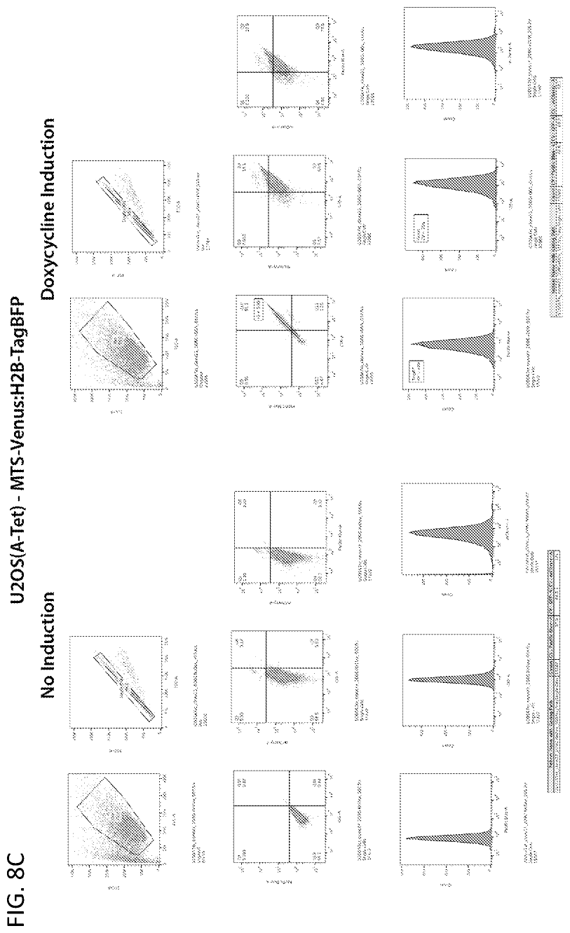

Only acceptor cell lines that pass all the checkpoints are selected. An example of the results for one acceptor cell line is shown in FIGS. 8A-8C.

### Example 5

### Optimization of the Multicistronic Platform

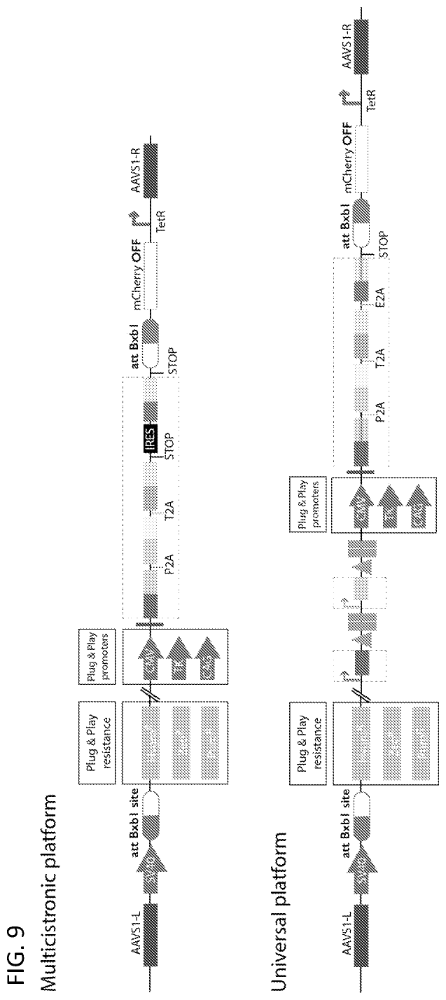

Several variants of the multicistronic platform have been developed, including one that comprises (1) plug-and-play recombinase sites; attB site for B×b1 specific serine-type recombination with the attP sequence in the acceptor cell lines and FRT site for recombination using the flippase (frt) of the tyrosine family site-specific recombinases; (2) plug-and-play promoter; (3) plug-and-play resistance marker; (4) three 2A peptidase sequences upstream of an IRES sequence to express up to four genes from the same promoter (FIG. 18). While the multicistronic platform contained a single transcript unit (TU) where all transcripts are driven by a single promoter; the universal platform included the option of integrating up to two of three different TUs by addition of core insulator sequences (chicken hypersensitivity site four core elements) that avoid promoter interference in addition to the aforementioned multicistronic unit (FIG. 9, bottom panel).

Recombination of reporter vectors was achieved with high efficiency by co-transfection with highly efficient serine-recombinase Bxbl (Duportet, X. et al., NAS (2014)). Recombination was accompanied by mCherry transcription loss due to its displacement from the ‘acceptor site’ promoter.

### Example 6

### Multiplexed High-Content Assay to Measure Cellular Stress Mechanisms for Predictive Toxicology Studies

Toxicity is a major concern for the pharmaceutical industry as well as environmental chemicals. Both drug candidates and approved drugs face the issue of induced cytotoxicity. New high-throughput assay platform that rapidly pinpoints compound mechanism of action and toxicity liabilities and monitor multiple facets of toxicity are needed. Described herein is a panel of multicolor cell stress reporters stably integrated into cell lines relevant for compound toxicity evaluation. The resulting panel of multicolor stable reporter cell lines can be used as the foundation for a toxicity multiplexed assay.

U2OS and HepG2 acceptor cells were transfected with the vector containing the recombinase Bxbl and a vector constitutively expressing TagBFP ('tester-reporter') to determine the best recombination conditions: transfection method; ratio recombinase:reporter; drug selection addition; cell plating number. The tester-reporter vector allows rapid quantification of transfection efficiency and recombination efficiency by flow cytometry analysis (FACS).

Transfection efficiency is calculated by the number of TagBFP+cells over the total population. Recombination efficiency is calculated by the ratio between number of cells with site-specific integration (mCherry- and TagBFP+) and the total number of cells that were transfected (TagBFP+). The cells where site-specific recombination occurred will be TagBFP+because the TagBFP will be expressed from the CAG promoter from the tester-reporter and will be mCherry-because recombination of the acceptor site will displace its start codon. Several conditions were tested.

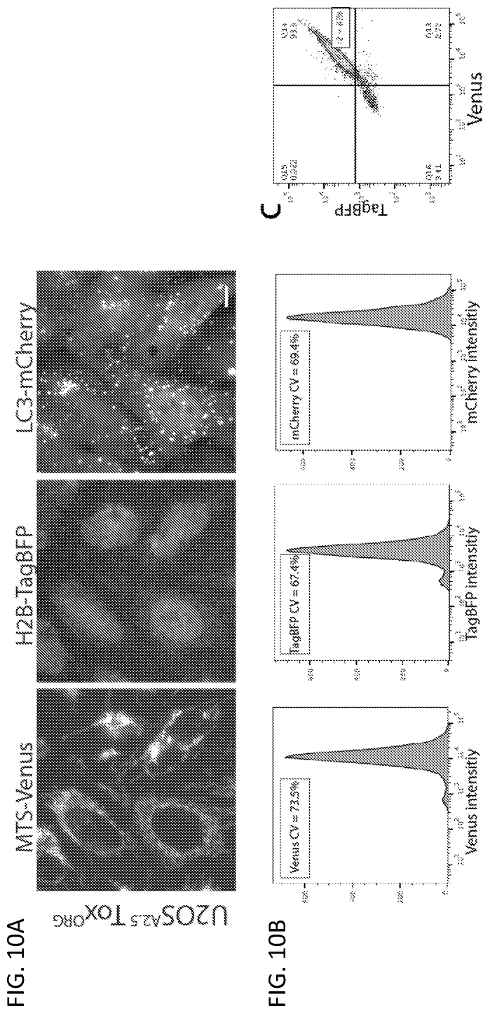

U2OS acceptor cells were co-transfected a construct containing the recombinase Bxbl and a multicistronic Tox reporter (named U2OS^{A}:Tox^{ORG}) carrying three fluorescently labeled proteins: MTS-Venus, H2B-TagBFP and mCherry-LC3 (FIG. 10A). After a 3-day treatment with hygromycin 3 days after the transfection, the surviving cells were grown for 10 additional days without any selection before FACS analysis. As desired, we obtained highly homogenous expression of the three fluorescent proteins. We observed that the variability of expression in the polyclonal population after integration measured by the coefficient of variation (CV) (FIG. 10B mCherry:=69%; TgBFP: CV=67%, Venus: CV=73%) is similar to that of the monoclonal chassis cell line population (mCherry: CV=68%). We also observed that expression of transgenes placed on the same payload is highly correlated (coefficient of correlation r=0.81, FIG. 10C).

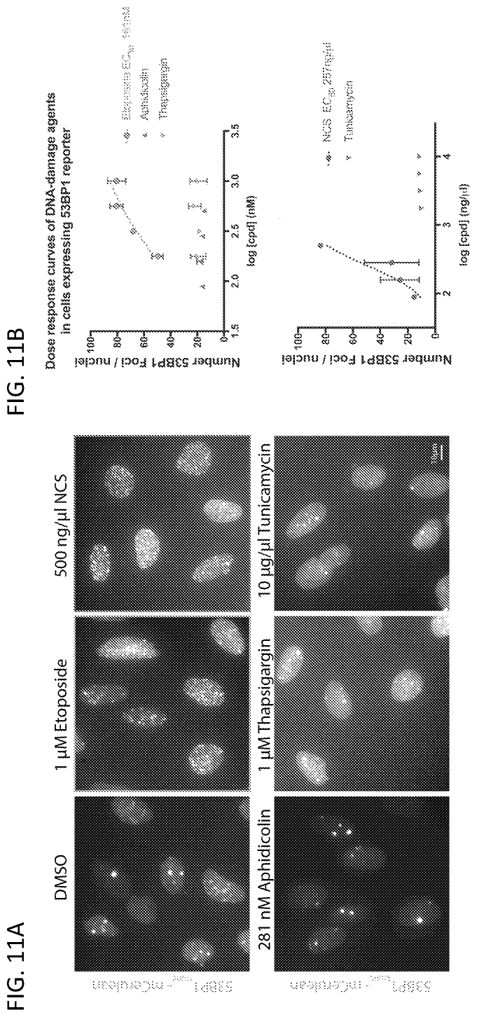

U2OS acceptor cells were co-transfected a construct containing the recombinase B×b1 and a mutlicistronic Tox reporter (named U2OS^{A}:Tox^{DUPR}) carrying three fluorescently labeled proteins: 53BP1-mCerulean that binds to DNA double strand breaks, H2B-mCherry and XBP1Δ-Venus which is a UPR stress related sensor. Once the cells were selected and expanded, they were assayed to determine the dose responses of etoposide, and neocarzinostatin (NCS). Etoposide inhibits DNA synthesis and is very active against cells in the late S and G2 phases of the cell cycle, induces double and single strand breaks in DNA in intact cells. NCS inhibits DNA synthesis, G2 cycle arrest, apoptosis. Both compounds are inducers of DNA damage and consequently increase the number of 53BP1 nuclear foci. Cells were plated in glass bottom well 384 well plates. To evaluate the specificity of the reporter, together with DNA inducers, three other compounds were used as controls. Thapsigargin, which induced UPR-related stress by inhibiting calcium uptake into ER (Ca²⁺ is chaperone cofactor so protein folding is inhibited), tunicamycin that is also involved in UPR-related stress by inhibiting glycosylation of newly synthesized proteins, and aphidicolin which inhibits DNA replication in eukaryotes. All compounds were tested in tetraplicates in four ten-fold serial dilutions with data collection 8 hour after compound addition. Images were acquired in a Nikon Ti-E inverted Microscope equipped with motorized stage with autofocus and OkoLab stage top environmental control chamber (supplied with CO₂ and temperature controlled for live cell imaging) (FIG. 11A).

The number of 53BP1-mCerulean was used as a metric for double strand breaks and DNA damage. For each assay, the images were analyzed using the open source software CellProfiler for quantification of the number of foci per nuclei and the data generated was further processed to determine compound EC_{50S}.

Image analysis was conducted using the open source software CellProfiler. After importing the images, nuclei were identified from the H2B-mCherry images as primary objects, followed by enhancement of mCerulean structures, and detection of these enhanced structures as objects. The nuclei and the foci are related and the number of children (foci) per nuclei is counted. Data from CellProfiler was exported to Python programming language for further computation.

Using Python, the data was grouped by well and the average number of foci/nuclei determined. The wells that corresponded to the same conditions were grouped. All the data was exported to GraphPad Prism 7.0 software to generate plots. A non-linear regression was used to fit the data and to calculate the concentration where we observe 50% activity of the enhancer (EC_{50S}) (FIG. 11B)

Etoposide activity was detected by U2OS^{A}:Tox^{DUPR} reporter cells (FIGS. 11A and 11B) with EC₅₀ value of 161 nM.

NCS activity was detected by U2OS^{A}:Tox^{DUPR} reporter cells (FIGS. 11A and 11B) with EC50 value of 257 ng/ml.

Tunicamycin, thapsigargin and aphidicolin do not show any effect in the number of 53BP1 foci, demonstrating that the number of 53BP1 foci is specifically correlated to DNA damage.

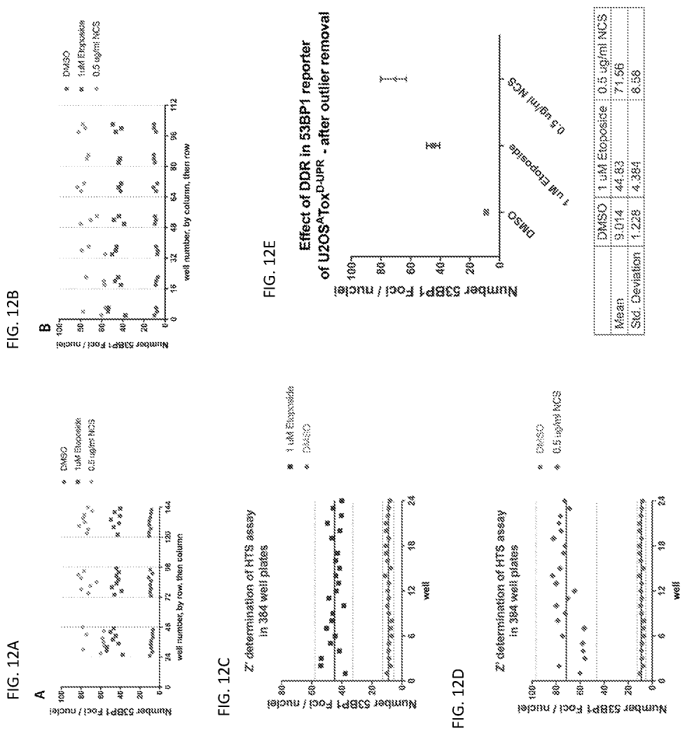

53BP1-mCerulean included in the U2OS^{A}:Tox^{DUPR} reporter cells is a functional and specific reporter for DNA damage and double strand breaks. The performance metrics of the U2OSA:Tox^{DUPR} reporter cells confirms that the reporter and the assay are suitable for high throughput assays (FIGS. 12A-12E). Z factor (Z′) of the assay is>0.5 validating its applicability for high throughput quantitative assays to monitor, measure and compare compound activity, dose response and determine EC_{50S} of compounds that induce DNA damage.

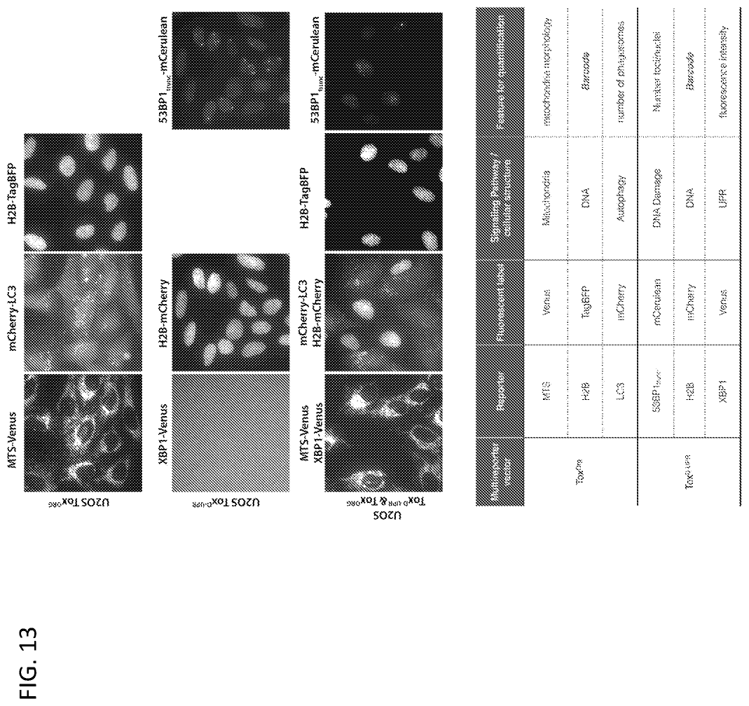

U2OS^{A}:Tox^{DUPR} and U2OS^{A}:Tox^{ORG} were combined in a pooled assay with an aggregate of 5 biosensors and phenotypic readouts (MTS-Venus, H2B-TagBFP, mCherry-LC3, 53BP1-cerulean, XBP1-Venus and H2B-mCherry) where DNA is differently labeled in each of the two reporter cell lines (FIG. 13).

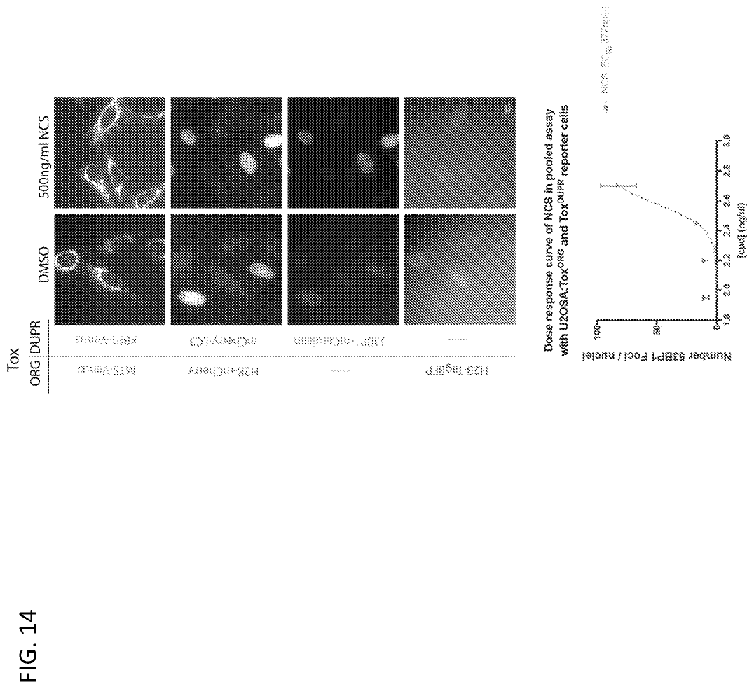

The two U2OS^{A} Tox^{ORG} and Tox^{DUPR} reporter cell lines were mixed 1:1 and plated in 384 well plates 24 h before compound addition. Two DNA damage inducers were tested: etoposide, and neocarzinostatin (NCS). Etoposide inhibits DNA synthesis and is very active against cells in the late S and G2 phases of the cell cycle, induces double and single strand breaks in DNA in intact cells. NCS inhibits DNA synthesis, G2 cycle arrest, apoptosis. Both compounds are inducers of DNA double strand breaks and consequently increase the number of 53BP1 nuclear foci. Eight hours post compound addition, images were acquired in a Nikon Ti-E inverted Microscope equipped with motorized stage with autofocus and OkoLab stage top environmental control chamber (supplied with CO₂ and temperature controlled for live cell imaging) (FIG. 14). The increase in the number of foci/nuclei specifically in Tox^{DUPR} reporter cells is observed in wells treated with NCS when compared to vehicle control wells. Similar EC₅₀ is obtained if reporter cells are assayed by itself (FIG. 11B, EC₅₀NCS=257 ng/μl) or pooled with other reporter cell lines (FIG. 14 EC₅₀NCS=376 ng/μl).

The pooled assay demonstrates that mixing reporter cell lines with different reporters allows to specifically infer the mechanisms of compound toxicity.

Three 3-color reporter vectors encompassing three stress signaling pathways (unfolded protein response, DNA damage, oxidative stress and p53-dependent stress response) with an aggregate of seven biosensors and phenotypic readouts that can be used separately or pooled to fingerprint mechanisms of compound toxicity were developed.

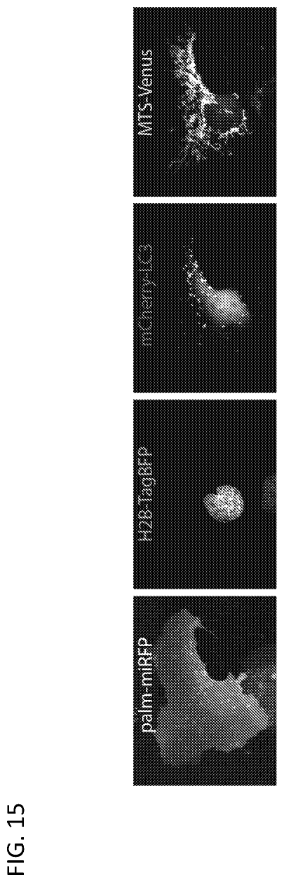

Additional platform for toxicity compound evaluation was developed. One four-color reporter vector encompassing four phenotypic markers (FIG. 15) (DNA, mitochondria, plasma membrane and autophagosomes) that can be used separately or pooled to fingerprint mechanisms of compound toxicity were developed and tested using the process described above.

### Example 7

### Generation of Multicolor iPS Cell Lines

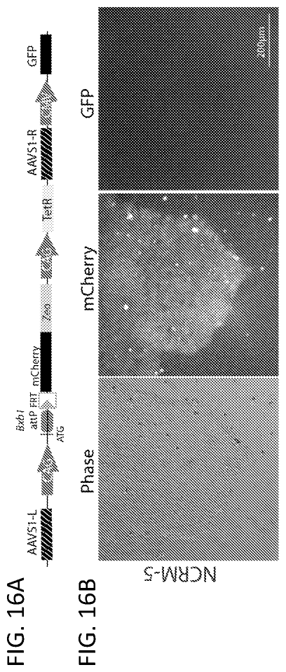

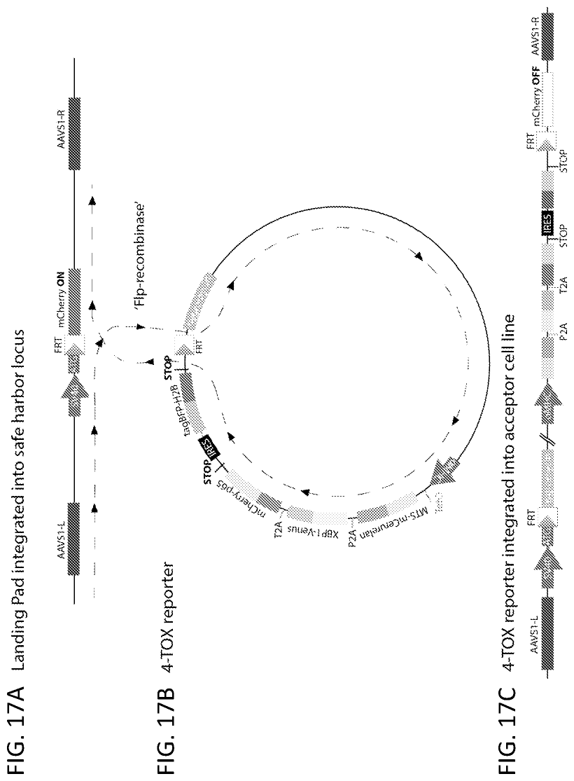

A robust targeting strategy using RNA-guided genome engineering tools mediated by Cas9 to introduce an ‘acceptor site’ (FIG. 16A) into the endogenous AAVS1 locus of iPSC lines was developed. The acceptor site contained: (1) an attP site to access recombination by serine recombinase Bxb1; (2) an mCherry fluorescence marker to confirm acceptor site integration; (3) an antibiotic resistance gene driven by the cytomegalovirus/chicken β-Actin promoter (CAG) promoter to enable cell selection; and (4) a GFP gene driven by the CMV promoter localized downstream of the homologous-recombination region to enable prompt distinction between random and targeted integrations (cells with random integration would fluoresce green due to GFP expression, while cells with targeted integration would not due to loss of CMV-GFP) (see FIG. 16B). After antibiotic selection, clones were generated from single cells and manually isolated. PCR was used to identify single allele integration of the acceptor site at the correct locus in these clones followed by droplet digital PCR to confirm single-copy integration (Table 3). PCR-based identification of integration is commonly used in the art and the process is well known and routine. This strategy was used to generate a panel of human immortalized acceptor cells, as explained in examples 1-3, and to establish two iPSC acceptor lines with single-copy integration of the acceptor site (Table 3).

TABLE 3

| | | N clones single | N clones | |
|---|---|---|---|---|
| | | allele integra- | with | % of clones with |
| | N clones | tion at the | 1 copy | single-copy at |
| | screened | AAVS1 locus | (ddPCR) | the AAVS1 locus |
| WTC | 24 | 21 | 12 | 50% |
| NCRM5 | 21 | 11 | 4 | 9% |

1. A multicistronic reporter vector comprising:
a promoter operably linked to an open reading frame, wherein the open reading frame comprises two or more cistrons, and wherein expression of the open reading frame in a cell yields separate component polypeptide products from each cistron;
wherein each cistron comprises a multiple cloning site (MCS) and nucleic acid encoding a reporter polypeptide, and wherein each cistron encodes a different reporter polypeptide; and
wherein expression of two or more nucleic acids encoding polypeptides inserted into the two or more multiple cloning sites is essentially stoichiometric.

2. The multicistronic reporter vector of claim 1, wherein the cistrons are separated from one another by a nucleic acid encoding one or more self-cleaving peptide and/or one or more internal ribosome entry site (IRES).

3. (canceled)

4. The multicistronic reporter vector of claim 2, wherein the one or more self-cleaving peptides is one or more 2A peptides.

5. The multicistronic reporter vector of claim 4, wherein one or more 2A peptides is a T2A peptide, a P2A peptide, an E2A peptide or a F2A peptide.

6. The multicistronic reporter vector of claim 1, wherein the reporter polypeptide further comprises one or more nucleic acids encoding a peptide linker between one or more of the reporter polypeptides and one or more of the self-cleaving peptides.

7. (canceled)

8. The multicistronic reporter vector of claim 1, wherein the reporter polypeptide is a fluorescent reporter polypeptide.

9. The multicistronic reporter vector of claim1, wherein the reporter polypeptide for each cistron is selected from GFP, EGFP, Emerald, Citrine, Venus, mOrange, mCherry, TagBFP, mTurquoise, Cerulean, UnaG, dsRed, eqFP611, Dronpa, RFP, TagRFPs, TdTomato, KFP, EosFP, Dendra, IrisFP, iRFPs, and smURFP.

10. The multicistronic reporter vector of claim 1, wherein the vector comprises
a) a promoter operably linked to an open reading frame, wherein the open reading frame comprises a first cistron and a second cistron, wherein each cistron comprises 5′ to 3′ nucleic acid comprising a MCS, nucleic acid encoding a reporter polypeptide, and nucleic acid encoding a linker peptide; wherein the first cistron and the second cistron are separated by nucleic acid encoding a viral cleavage peptide
b) a promoter operably linked to an open reading frame, wherein the open reading frame comprises a first cistron, a second cistron and a third cistron, wherein each cistron comprises 5′ to 3′ nucleic acid comprising a MCS, nucleic acid encoding a reporter polypeptide, and nucleic acid encoding a linker peptide; wherein the first cistron and the second cistron are separated by nucleic acid encoding a first viral cleavage peptide and the second cistron and the third cistron are separated by nucleic acid encoding a second viral cleavage peptide;
c) a promoter operably linked to an open reading frame, wherein the open reading frame comprises a first cistron, a second cistron, a third cistron and a fourth cistron, wherein each cistron comprises 5′ to 3′ nucleic acid comprising a MCS, nucleic acid encoding a reporter polypeptide nucleic acid encoding a linker peptide; wherein the first cistron and the second cistron are separated by nucleic acid encoding a first viral cleavage peptide, the second cistron and the third cistron are separated by nucleic acid encoding a second viral cleavage peptide the third cistron and the fourth cistron are separated by nucleic acid encoding a third viral cleavage peptide; or
d) a promoter operably linked to an open reading frame, wherein the open reading frame comprises a first cistron, a second cistron, a third cistron and a fourth cistron, wherein each cistron comprises 5′ to 3′ nucleic acid comprising a MCS, nucleic acid encoding a reporter polypeptide, nucleic acid encoding a linker peptide; wherein the first cistron and the second cistron are separated by nucleic acid encoding a first viral cleavage peptide, the second cistron and the third cistron are separated by nucleic acid encoding a second viral cleavage peptide the third cistron and the fourth cistron are separated by nucleic acid encoding an IRES.

11-cm 13. (canceled)

14. The multicistronic reporter vector of claim 1, wherein the vector further comprises one or more inducible elements located between the promoter and open reading frame.

15. (canceled)

16. The multicistronic reporter vector of claim 14, wherein the inducible element is a Tet operator 2 (TetO2) inducible element.

17. The multicistronic reporter vector of claim 1, wherein the promoter is a constitutive promoter.

18. The multicistronic reporter vector of claim 17 wherein the constitutive promoter is a Cytomegalovirus a (CMV), a Thymidine Kinase (TK), an eF1-alpha, a Ubiquitin C (UbC), a Phosphoglycerate Kinase (PGK), a CAG promoter, an SV40 promoter, a human β-actin promoter or a tissue-specific promoter.

19. The multicistronic vector of claim 1, wherein the promoter is an inducible promoter.

20. The multicistronic vector of claim 19, wherein the inducible promoter is a tetracycline responsive promoter.

21. (canceled)

22. The multispecific reporter vector of claim 18, wherein the promoter is tissue specific promoter specific for cells of heart, blood, muscle, lung, liver, kidney, pancreas, brain, or skin.

23. The multicistronic reporter vector of claim , further comprising a site-specific recombinase sequence located 3′ to the open reading frame.

24. The multicistronic reporter vector of claim 23, wherein the vector further comprises nucleic acid encoding a selectable marker, wherein the nucleic acid encoding the selectable marker is not operably linked to the promoter when the site-specific recombinase sequence has not recombined and is operably linked to the promoter when the site-specific recombinase sequence recombines with its target site-specific recombinase sequence.

25. The multicistronic reporter vector of claim 24, wherein the site-specific recombinase sequence is a FRT nucleic acid sequence and/or an attP nucleic acid and/or a loxP nucleic acid sequence.

26-27.(canceled)

28. The multicistronic reporter vector of claim 1, wherein the one or more polypeptides comprise polypeptides that can be used to profile a single biological pathway, cross-talk between two or more biological pathways, synthetic lethality, cellular homeostasis, organelle homeostasis or a toxicity response.

29. The multicistronic reporter vector of claim 1, further comprising one, two or three transcription units comprising a promoter and nucleic acid encoding a transgene located 5′ to the open reading frame comprising two or more cistrons, wherein the reporter vector further comprises a core insulator sequence and a polyA sequence located 3′ to the transcription units and 5′ to the open reading frame comprising two or more cistrons.

30. An acceptor cell for receiving a multicistronic reporter vector, wherein the acceptor cell comprises a recombinant nucleic acid integrated into a specific site in a host cell genome, wherein the recombinant nucleic acid comprises a first promoter operably linked to nucleic acid encoding a fusion polypeptide, wherein the fusion polypeptide comprises a reporter domain and a selectable marker domain, and wherein the nucleic acid comprises a site-specific recombinase nucleic acid sequence located at the 5′ end of the nucleic acid encoding the fusion polypeptide.

31-46. (canceled)

47. A method for generating an acceptor cell for receiving a multicistronic reporter vector, the method comprising introducing a recombinant nucleic acid to a cell wherein the recombinant nucleic acid comprises 5′ to 3′
a) a first nucleic acid for targeting homologous recombination to a specific site in the cell,
b) a first promoter,
c) site-specific recombinase nucleic acid,
d) nucleic acid encoding a first reporter polypeptide and a selectable marker,
e) a second nucleic acid for targeting homologous recombination to a specific site in the cell,
f) a second promoter and nucleic acid encoding a second reporter polypeptide,
wherein expression of the first reporter polypeptide without expression of the second reporter polypeptide indicates targeting integration of the recombinant nucleic acid to the specific site in the cellular genome and expression of the first and second reporter polypeptides indicates random integration in the cellular genome.

48-65. (canceled)

66. A multireporter cell comprising a multicistronic reporter construct, wherein the multicistronic reporter construct comprises
a promoter operably linked to an open reading frame, wherein the open reading frame comprises two or more cistrons;
wherein each cistron comprises a nucleic acid encoding a different transgene product fused to a different reporter polypeptide, wherein expression of the open reading frame in a cell yields separate component polypeptide products from each cistron; and
wherein expression of the transgene products is essentially stoichiometric.

67-85. (canceled)

86. A method for generating a multireporter cell, the method comprising introducing the multicistronic reporter vector into the acceptor cell of claim 33; wherein the multicistronic reporter vector comprises
a promoter operably linked to an open reading frame, wherein the open reading frame comprises two or more cistrons, and wherein expression of the open reading frame in a cell yields separate component polypeptide products from each cistron;
wherein each cistron comprises a multiple cloning site (MCS) and nucleic acid encoding a reporter polypeptidevector, and wherein each cistron encodes a different reporter polypeptide;
and
wherein expression of two or more nucleic acids encoding polypeptides inserted into the two or more multiple cloning sites is essentially stoichiometric.

87-89. (canceled)

90. A library of multireporter vectors, wherein the library comprises multicistronic reporter vectors comprise nucleic acid encoding different transgene products fused to reporter polypeptides of claim 1, wherein two or more of the different transgene products on each vector are expressed essentially stoichiometrically when introduced to cells.

91. The library of claim 90, wherein the reporter vectors encode two or more transgenes encode polypeptides that can be used to profile a single biological pathway, cross-talk between two or more biological pathways, cellular homeostasis, organelle homeostasis or a toxicity response.

92-97. (canceled)

98. A library of acceptor cells for receiving multicistronic reporter vectors, wherein the library comprises acceptor cells according to claim 30.

99. A library of multireporter cells, wherein each cell in the library comprises multicistronic reporter vector comprising nucleic acids encoding different transgene products fused to reporter polypeptides, wherein the different nucleic acids encoding different transgene products on each vector are expressed essentially stoichiometrically when introduced to cells.

100-109. (canceled)

110. The library of cells of claim 99, wherein the reporter vectors encode one or more transgenes one or more polypeptides comprise polypeptides that can be used to profile a single biological pathway, cross-talk between two or more biological pathways, cellular homeostasis, or phenotypic features.

111-116. (canceled)

117. A kit comprising one or more multicistronic reporter vectors of claim 1.

118. A kit comprising one or more acceptor cells of claim 30.

119. (canceled)

120. A kit comprising one or more multireporter cells of claim 64.

121-122. (canceled)

123. A method of profiling two or more polypeptides in a live cell, the method comprising determining the expression of the two or more of the transgenes and/or location of the two or more transgene products of a multireporter cell of claim 64.

124. The method of claim 123, wherein the method is used to profile a single biological pathway, cross-talk between two or more biological pathways, synthetic lethality, cellular homeostasis, organelle homeostasis or a toxicity response.

125-130. (canceled)

131. A method of measuring the effects of an agent on the profile of two or more polypeptides in a live cell, the method comprising subjecting a multireporter cell of claim 64 to the agent and determining the expression of the two or more of the transgenes and/or location of the two or more transgene products in the cell in response to the agent.

132-136. (canceled)
